(19) United States
(12) Patent Application Publication
(10) Pub. No.: US 2021/0371860 A1
(43) Pub. Date: Dec. 2, 2021

(54) OLIGONUCLEOTIDES FOR MODULATING MYH7 EXPRESSION

(71) Applicant: Roche Innovation Center Copenhagen A/S, Hørsholm (DK)

(72) Inventors: Peter HAGEDORN, Hørsholm (DK); Richard OLSON, Cambridge, MA (US); Marianne JENSEN, Køge (DK); Annapurna PENDRI, Princeton, NJ (US); Brian ANDERSON, Wallingford, CT (US); Sean Carl LITTLE, Pipersville, PA (US); Linda Jane BRISTOW, Boston, MA (US); John Ryan THOMPSON, Scotch Plains, NJ (US); Ron AMMAR, Pennington, NJ (US)

(73) Assignee: Roche Innovation Center Copenhagen A/S, Hørsholm (DK)

(21) Appl. No.: 17/053,741

(22) PCT Filed: May 7, 2019
(86) PCT No.: PCT/EP2019/061718

§ 371 Date: Nov. 6, 2020

Related U.S. Application Data
(60) Provisional application No. 62/668,722, filed on May 8, 2018.

(51) Int. Cl. C12N 15/113

(52) U.S. Cl. CPC C12N 15/113; C12N 2310/11; C12N 2310/3231; C12N 2310/341; C12N 2310/3341; C12N 2310/322; C12N 2310/321; C12N 2310/315; C12N 2320/34

(57) ABSTRACT

The present invention relates to antisense oligonucleotides that are capable of modulating expression of MYH7 in a target cell. The oligonucleotides hybridize to MYH7 mRNA. The present invention further relates to conjugates of the oligonucleotide and pharmaceutical compositions and methods for treatment of hypertrophic cardiomyopathy using the oligonucleotide.

### FIELD OF INVENTION

The present invention relates to antisense oligonucleotides which target human myosin heavy chain 7 (MYH7) transcript. In some aspects, the oligonucleotides of the invention may be used to selectively inhibit the expression a disease associate allele of MYH7. Inhibition of MYH7 expression is beneficial for a range of medical disorders, including hypertrophic cardiomyopathy.

### BACKGROUND

Familial hypertrophic cardiomyopathy (HCM) is a monogenic disease clinically characterized by asymmetrical ventricular hypertrophy, arrhythmias, and progressive heart failure. HCM has a prevalence of 1:500 and about 40% of cases are due to autosomal dominant mutations in the MYH7 gene. MYH7 encodes the β-myosin heavy chain protein that acts as a molecular motor to drive active contraction during cardiac systole. More than 300 missense mutations in MYH7 have been linked to HCM pathology, and these mutations are distributed throughout the gene. There is no common mechanism that links each MYH7 mutation to the HCM phenotype; mutations can affect filament sliding velocity, ATPase rate, force, and calcium sensitivity of activation. Regardless of the exact mutation and its specific effect on actomyosin dynamics, the link between MYH7 mutation and HCM derives from mutant myosin protein that is expressed, stable, and exerts dominant negative effects.

Hundreds of dominant negative myosin mutations have been identified that lead to hypertrophic cardiomyopathy (HCM), and the biomechanical link between mutation and disease is heterogeneous across this patient demographic. This represents a major challenge for therapeutic intervention for the treatment or prevention of hypertrophic cardiomyopathy.

WO2015/042581 discloses a method of preventing or treating hypertrophic cardiomyopathy (HCM) in a subject having in their genome a first MYH7 allele comprising an HCM-causing mutation and a second MYH7 allele that does not comprise the HCM-causing mutation, the method comprising administering to the subject an interfering RNA molecule that selectively inactivates the transcript encoded by the first MYH7 allele compared to the transcript encoded by the second MYH7 allele. siRNAs targeting the T403Q mutation are disclosed.

WO 2015/113004 discloses a method for treating a subject having hypertrophic cardiomyopathy comprising administering a siRNA which selectively down-regulates expression of myosin heavy chain-403Q.

WO2016/149684 discloses a method for down-regulating disease causing alleles using RNAi therapeutics system, where subject samples are sequences to identify deleterious mutation on a particular allele as a common variant in phase with the deleterious mutation, and selecting a RNAi therapeutic targeting the common variant using the RNAi therapeutics system, and applying the selected RNAi therapeutics system utilizing a vector and the RNAi therapeutics system. The RNAi therapeutics system may include a 2′-O-methylated antisense nucleic acid phosphorothioate compound complementary to common variants of the Myh7 gene.

### OBJECTIVE OF THE INVENTION

The present invention identifies novel oligonucleotides which modulate MYH7, which may be used for allelic selective inhibition of MYH7.

### SUMMARY OF INVENTION

The present invention relates to oligonucleotides targeting a MYH7 nucleic acid which are capable of inhibiting the expression of MYH7.

The invention provides oligonucleotides which target the expression of a MYH7 allelic variant selected from a MYH7 allelic variant which comprises a single nucleotide polymorphism at a position selected from rs2239578, rs2069540, and rs7157716 (RefSNP see dbSNP, NCBI Homo sapiens Annotation Release 109, 2018-03-27, hereby incorporated by reference). These three common SNPs are found in intron 2, exon 3, and exon 24 of MYH7 pre-mRNA respectively, and are referred to as rs223, rs206, and rs715 herein.

In some embodiments the oligonucleotide of the invention selectively inhibits a MYH7 allelic variant, such as an allelic variant at a position of the human MYH7 transcript selected from rs223, rs206 and rs715.

The invention provides an antisense oligonucleotide targeting human myosin heavy chain 7 (Myh7) transcript, wherein said oligonucleotide comprises a contiguous nucleotide sequence of 10-30 nucleotides in length which are at least 90% complementary to a sequence selected from the group consisting of SEQ ID NOs 3-10.

The invention provides an antisense oligonucleotide targeting human myosin heavy chain 7 (Myh7) transcript, wherein said oligonucleotide comprises a contiguous nucleotide sequence of 10-30 nucleotides in length which are at least 90% complementary to a sequence selected from the group consisting of SEQ ID NOs 3 & 4, or SEQ ID NOs 5-10.

The invention provides an antisense oligonucleotide 10-40 nucleotides in length, targeting human myosin heavy chain 7 (Myh7) transcript, wherein said oligonucleotide comprises a contiguous nucleotide sequence of 10-30 nucleotides in length which are at least 90% complementary to a sequence selected from the group consisting of SEQ ID NOs 3 & 4, or SEQ ID NOs 5-10.

In some embodiments, the antisense oligonucleotide is complementary to a region of the sequence selected from SEQ ID NOs 3-10 wherein the region of complementarity comprises the 20^{th} nucleotide from the 5′ end of the sequence selected from SEQ ID NOs 3-10.

In some embodiments, the antisense oligonucleotide is a LNA modified oligonucleotide, such as an LNA gapmer.

The invention provides for a conjugate comprising the oligonucleotide according to the invention and at least one conjugate moiety covalently attached to said oligonucleotide.

The invention provides for a pharmaceutically acceptable salt of the antisense oligonucleotide or the conjugate according to the invention,

The invention provides for a pharmaceutical composition comprising the antisense oligonucleotide or the conjugate of the invention and a pharmaceutically acceptable diluent, solvent, carrier, salt and/or adjuvant.

The invention provides for a method for modulating human myosin heavy chain 7 (Myh7) expression in a target cell which is expressing Myh7, said method comprising administering an oligonucleotide of the invention or the conjugate of the invention or the pharmaceutically acceptable salt of the invention or the pharmaceutical composition of the invention in an effective amount to said cell. In some embodiments the method is in vivo. In some embodiments the method is in vitro.

The invention provides for a method for treating or preventing a disease comprising administering a therapeutically or prophylactically effective amount of an oligonucleotide of the invention or the conjugate of the invention or the pharmaceutically acceptable salt of the invention or the pharmaceutical composition of the invention to a subject suffering from or susceptible to the disease.

In some embodiments the disease is hypertrophic cardiomyopathy.

The invention provides for an oligonucleotide of the invention or the conjugate of the invention or the pharmaceutically acceptable salt of the invention or the pharmaceutical composition of the invention for use in medicine.

The invention provides for an oligonucleotide of the invention or the conjugate of the invention or the pharmaceutically acceptable salt of the invention or the pharmaceutical composition of the invention for use in the treatment or prevention of hypertrophic cardiomyopathy.

The invention provides for the use of the oligonucleotide of the invention or the conjugate of the invention or the pharmaceutically acceptable salt of the invention or the pharmaceutical composition of the invention, for the preparation of a medicament for treatment or prevention of hypertrophic cardiomyopathy.

The invention provides for a method for treatment of a human subject in need to treatment for hypertrophic cardiomyopathy, said treatment comprising the step of:

a. Taking a biological sample from the human subject

b. Sequencing the Myh7 nucleic acid alleles present in the sample of the human subject;

c. Determine the presence of a disease associated Myh7 allelic variant of the Myh7 nucleic acid;

d. Administer a therapeutically effective amount of an antisense oligonucleotide to the human subject which is selective for the disease associated Myh7 allelic variant as compared to a non-disease associate allele, such as the oligonucleotide of the invention or the conjugate of the invention or the pharmaceutically acceptable salt of the invention or the pharmaceutical composition of the invention.

### BRIEF DESCRIPTION OF FIGURES

FIG. 1. SNP Targeting strategy a) SNP heterozygosity across five genetic super populations. See http://www.internationalgenome.org/category/population/ for details on population descriptions. b) Developing ASOs for individual HCM mutations is not currently a feasible therapeutic strategy. By targeting SNPs, multiple MYH7 disease-causing mutations can be targeted with a single ASO.

FIG. 2. Evaluation of SNP-selective ASOs from initial library in skeletal muscle myoblast cell lines a) Example of concentration response curves for ASO A181 from the rs223-C sub-library showing high SNP-selectivity. Selectivity is defined as the IC₅₀ in SNP-mismatched cells divided by the IC₅₀ in SNP-matched cells. The vertical dashed lines indicate potencies estimated at 0.27 and 19 μM on matched and mismatched alleles, respectively, resulting in 70-fold selectivity. b-d) Potency and selectivity evaluated at day 10 are plotted for rs715, rs223, and rs206 ASOs from the initial library. ASOs targeting the C and T allele of a given SNP are shown as red and black dots, respectively. ASOs with selectivities >50-fold were all fixed at the same level on the y-axis. In the rs715 ASO plot b), the three diamond symbols indicate the ASOs selected for redesigns.

FIG. 3. Evaluation of SNP-selective ASOs from redesign library targeting the rs715 SNP. Potency and selectivity evaluated in a) human myoblast CC-2580 cells and b) human iPSC-derived cardiomyocytes. ASOs targeting the C and T allele of a given SNP are shown as red and black dots, respectively. Dots in pink and grey are parent ASOs from the initial library. The five diamond symbols indicate the ASOs selected for evaluation in mice. c) Correlation between potencies in CC-2580 cells and iPSC-CM cells. Significance of the correlation was determined by Spearman's rank correlation test.

FIG. 4. Time course study of SNP-selective knockdown. mRNA knockdown in human iPSC-derived cardiomyocytes was evaluated at six time points over a two-week period using allele-specific droplet digital PCR. At day 0, 250 nM of rs715-T targeting ASO A259 was added via gymnosis. SNP-matched knockdown is seen for up to two weeks, while the SNP-mismatched allele does not show knockdown. Data were normalized to the no ASO day 2 time point for each allele. Mean+/−SD from three independent experiments is shown. Significance between T alleles (no ASO vs ASO) was determined by two-way ANOVA followed by Sidak's multiple comparisons test (*p<0.05, ***p<0.001).

FIG. 5. Study of SNP-selective knockdown in mice. a) Allele-specific mRNA quantitation from mouse LV one week following ASO dosing (*p<0.05, **p<0.01, ***p<0.001 comparing C and T allele abundance within a group as determined by t-test). All five compounds significantly reduce the C allele compared to the T allele. Two compounds give significant knockdown of the C allele compared to the C allele in the saline group (^{###}p<0.001 comparing to saline C allele as determined by one-way ANOVA followed by Dunnett's multiple comparisons test). b) ASO concentrations in heart (LV), liver, and kidney. On average, ASO concentration is 37× higher in kidney and 16× higher in liver compared to heart.

FIG. 6. a) 46 LNA gapmer ASOs were designed to various regions of the human MYH7 transcript (i.e. not SNP targeting). A subset of ASOs show robust knockdown at a concentration of 5 uM in 8220 myoblasts, establishing proof of concept that ASOs could be used to reduce MYH7 mRNA levels in vitro. A positive control ASO (S17) was identified from this initial dataset. b) The S17 positive control ASO shows similar knockdown at 5 uM in both 8220 and NH10 human skeletal muscle myoblasts, the two SNP homozygous cell lines used in the QuantiGene screen. This result suggests similar ASO uptake between the cell lines.

FIG. 7a. 102 ASOs were redesigned based on the A250 sequence, which targets the rs715-C allele (TCagcttggcgatgATCT; LNA uppercase, DNA lowercase). The primary sequence was maintained, but the distribution of LNA and DNA bases was varied. SNP-matched (C allele) and SNP-mismatched (T allele) knockdown at 0.5 uM is shown. Data points lying above the dotted line indicate stronger C allele knockdown. A250 data is shown with a larger black circle.

FIG. 7b. 162 ASOs were redesigned based on the A270 sequence, which targets the rs715-T allele (CTtggcaatgatctcATCC; LNA uppercase, DNA lowercase). The primary sequence was maintained, but the distribution of LNA and DNA bases was varied. SNP-matched (T allele) and SNP-mismatched (C allele) knockdown at 0.5 uM is shown. Data points lying below the dotted line indicate stronger T allele knockdown. A270 data is shown with a larger black circle.

FIG. 7c. ASOs were redesigned based on the A249 sequence, which targets the rs715-C allele (CAGcttggcgatgatCT; LNA uppercase, DNA lowercase). The primary sequence was maintained, but the distribution of LNA and DNA bases was varied. SNP-matched (C allele) and SNP-mismatched (T allele) knockdown at 0.5 uM is shown. Data points lying above the dotted line indicate stronger C allele knockdown. A249 data is shown with a larger black circle.

FIG. 8. Quantification of β-MHC in iCell2 hiPSC-CM with and without addition of A259 (rs715-T targeting ASO). β-MHC is not reduced at any timepoint, suggesting protein compensation by the SNP-mismatched allele. Bar graphs from n=3 independent experiments. Protein levels were normalized to the no ASO group at each timepoint.

FIG. 9. A small section of human MYH7 containing the rs715-C SNP and flanking sequence was inserted into one allele of the mouse Myh6 gene. The SNP base is shown in green. The other Myh6 allele was unchanged. This insertion of human sequence did not affect amino acid sequence. Five ASOs targeting the rs715-C allele were tested in these partially humanized mice. Because of the presence of an additional mismatch between human MYH7 and mouse Myh6 near the SNP position, all ASOs have two basepair mismatches between their template sequence and the WT Myh6 sequence.

FIG. 10. Weights and clinical chemistry following MYH7 ASO administration. No differences were seen in body weight change or heart weight/body weight. No difference was seen in the kidney injury marker BUN, but ASO B44 caused increased creatinine compared to vehicle. Liver injury markers (ALT, AST, AlkPhos) were increased following B44 and B56 dosing. *p<0.05, **p<0.01, ***p<0.001 compared to vehicle using one-way ANOVA and Dunnett's multiple comparisons test.

### DEFINITIONS

Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides, such as 2′ sugar modified nucleosides.

Antisense Oligonucleotides

The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self complementarity is less than 50% across of the full length of the oligonucleotide.

Advantageously, the single stranded antisense oligonucleotide of the invention does not contain RNA nucleosides, since this will decrease nuclease resistance.

Advantageously, the antisense oligonucleotide of the invention comprises one or more modified nucleosides or nucleotides, such as 2′ sugar modified nucleosides. Furthermore, it is advantageous that the nucleosides which are not modified are DNA nucleosides.

Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence, such as a F-G-F′ gapmer region, and may optionally comprise further nucleotide(s), for example a nucleotide linker region which may be used to attach a functional group to the contiguous nucleotide sequence. The nucleotide linker region may or may not be complementary to the target nucleic acid.

Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

Modified Internucleoside Linkages

The term “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides, such as region F and F′.

In an embodiment, the oligonucleotide comprises one or more internucleoside linkages modified from the natural phosphodiester, such one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 80 or such as at least 90% of the internucleoside linkages in the oligonucleotide, or contiguous nucleotide sequence thereof, are nuclease resistant internucleoside linkages. In some embodiments all of the internucleoside linkages of the oligonucleotide, or contiguous nucleotide sequence thereof, are nuclease resistant internucleoside linkages. It will be recognized that, in some embodiments the nucleosides which link the oligonucleotide of the invention to a non-nucleotide functional group, such as a conjugate, may be phosphodiester.

A preferred modified internucleoside linkage is phosphorothioate.

Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, which the internucleoside linkage in region G may be fully phosphorothioate.

Advantageously, all the internucleoside linkages in the contiguous nucleotide sequence of the oligonucleotide are phosphorothioate linkages.

It is recognized that, as disclosed in EP2 742 135, antisense oligonucleotide may comprise other internucleoside linkages (other than phosphodiester and phosphorothioate), for example alkyl phosphonate/methyl phosphonate internucleosides, which according to EP2 742 135 may for example be tolerated in an otherwise DNA phosphorothioate the gap region.

Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In a some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

Modified Oligonucleotide

The term modified oligonucleotide describes an oligonucleotide comprising one or more sugar-modified nucleosides and/or modified internucleoside linkages. The term chimeric” oligonucleotide is a term that has been used in the literature to describe oligonucleotides with modified nucleosides.

Complementarity

The term “complementarity” describes the capacity for Watson-Crick base-pairing of nucleosides/nucleotides. Watson-Crick base pairs are guanine (G)-cytosine (C) and adenine (A)—thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

The term “fully complementary”, refers to 100% complementarity.

Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

Target Nucleic Acid

According to the present invention, the target nucleic acid is a nucleic acid which encodes human MYH7 and may for example be a gene, a RNA, a mRNA, and pre-mRNA, a mature mRNA or a cDNA sequence. The target may therefore be referred to as an MYH7 target nucleic acid. In some embodiments, the target nucleic acid is selected from the group consisting of SEQ ID NO: 1, and SEQ ID NO 2, or naturally occurring variants thereof (e.g. sequences encoding a human MYH7 protein. In some embodiments, the target nucleic acid is an allelic variant of the human MYH7 transcript. In some embodiment

In some embodiments the target nucleic acid is a MYH7 allelic variant which comprises a polymorphism in at a position of the human MYH7 transcript selected from rs223, rs206 and rs715.

In some embodiments the polymorphism is selected from rs223T or rs223C.

In some embodiments the polymorphism is selected from rs206C or rs206T.

In some embodiments the polymorphism is selected from rs715C or rs715T.

In some embodiments the oligonucleotide on the invention selectively inhibits the target nucleic acid as compared to an alternative allelic variant of the target nucleic acid. The target nucleic acid and the alternative allelic variant comprise a single nucleotide polymorphism within the region which is complementary to the oligonucleotide of the invention or contiguous nucleotide sequence thereof. Selective inhibition refers to a higher inhibitory activity (higher potency) against the target nucleic acid as compared to the allelic variant. Selective inhibition can be determined in vitro (IC50) or in vivo (e.g. ED50).

If employing the oligonucleotide of the invention in research or diagnostics the target nucleic acid may be a cDNA or a synthetic nucleic acid derived from DNA or RNA.

For in vivo or in vitro application, the oligonucleotide of the invention is typically capable of inhibiting the expression of the MYH7 target nucleic acid in a cell which is expressing the MYH7 target nucleic acid. The contiguous sequence of nucleobases of the oligonucleotide of the invention is typically complementary to the MYH7 target nucleic acid, as measured across the length of the oligonucleotide, optionally with the exception of one or two mismatches, and optionally excluding nucleotide based linker regions which may link the oligonucleotide to an optional functional group such as a conjugate, or other non-complementary terminal nucleotides (e.g. region D′ or D″). The target nucleic acid may, in some embodiments, be a RNA or DNA, such as a messenger RNA, such as a mature mRNA or a pre-mRNA. In some embodiments the target nucleic acid is a RNA or DNA which encodes mammalian MYH7 protein, such as human MYH7, e.g. the human MYH7 mRNA sequence, such as that disclosed as SEQ ID NO 1 or 2.

TABLE 1
Genome and assembly information for MYH7.

| | | | | | | NCBI reference |
|---|---|---|---|---|---|---|
| | | | Genomic coordinates | | | sequence* accession |
| Species | Chr. | Strand | Start | End | Assembly | number for mRNA |
| Human | 14 | Rv | 23412738 | 23435718 | GRCh38 | NM_000257 |

Fwd = forward strand. Rv = reverse strand. The genome coordinates provide the pre-mRNA sequence (genomic sequence). The NCBI reference provides the mRNA sequence (cDNA sequence).
*The National Center for Biotechnology Information reference sequence database is a comprehensive, integrated, non-redundant, well-annotated set of reference sequences including genomic, transcript, and protein. It is hosted at www.ncbi.nlm.nih.gov/refseq.

TABLE 2
Sequence details for human MYH7.

| | | | |
|---|---|---|---|
| Species | RNA type | Length (nt) | SEQ ID NO |

| | | |
|---|---|---|
| Human | premRNA | 1 |
| Human | mRNA | 2 |

Target Sequence

The term “target sequence” as used herein refers to a sequence of nucleotides present in the target nucleic acid which comprises the nucleobase sequence which is complementary to the oligonucleotide of the invention. In some embodiments, the target sequence consists of a region on the target nucleic acid with a nucleobase sequence that is complementary to the contiguous nucleotide sequence of the oligonucleotide of the invention. This region of the target nucleic acid may interchangeably be referred to as the target nucleotide sequence, target sequence or target region. In some embodiments the target sequence is longer than the complementary sequence of a single oligonucleotide, and may, for example represent a preferred region of the target nucleic acid which may be targeted by several oligonucleotides of the invention.

In some embodiments the target sequence is a sequence selected from the group consisting of SEQ ID NO 3-10.

TABLE 3
Human MYH7 SNP regions.

| SEQ ID NO | SNP ID | Sequence | Allele |
|---|---|---|---|
| 3 | rs223-t | AGAAAAGCTGAAGCTAGAGTGTTGAAAATCTAGTAAGAC | REF |
| 4 | rs223-c | AGAAAAGCTGAAGCTAGAGCGTTGAAAATCTAGTAAGAC | ALT |
| 5 | rs206-c | GCAAAGTCACTGCCGAGACCGAGTATGGCAAGACAGTGA | REF |
| 6 | rs206-t | GCAAAGTCACTGCCGAGACTGAGTATGGCAAGACAGTGA | ALT |
| 7 | rs206-c-pre | GCAAAGTCACTGCCGAGACCGAGTATGGCAAGGTGGGTG | REF |
| 8 | rs206-t-pre | GCAAAGTCACTGCCGAGACTGAGTATGGCAAGGTGGGTG | ALT |
| 9 | rs715-t | CTGGGCTGGATGAGATCATTGCCAAGCTGACCAAGGAGA | REF |
| 10 | rs715-c | CTGGGCTGGATGAGATCATCGCCAAGCTGACCAAGGAGA | ALT |

The SNP positions are underlined.
REF = Reference. ALT = Alternative.

The bold underlined residue identifies a single nucleotide polymorphism (SNP) which the oligonucleotides of the invention may target (either the REF or ALT may be present in the target nucleic acid) REF refers to the designated wildtype allele of the highlighted SNP, ALT refers to an allelic variant. The respective location of SEQ ID NO 3-10 on the human MYH7 transcript sequences SEQ ID NO 1 or 2 are illustrated in table 4:

TABLE 4
Positions of human MYH7 SNP regions in the MYH7 mRNA and pre-mRNA

| | | SEQ ID NO 1 | SEQ ID NO 1 | SEQ ID NO 2 | SEQ ID NO 2 |
|---|---|---|---|---|---|
| SEQ ID NO | SNP ID | start | end | start | end |
| 3 | rs223-t | 1529 | 1567 | | |
| 4 | rs223-c | 1529 | 1567 | | |
| 5 | rs206-c | | | 301 | 339 |
| 6 | rs206-t | | | 301 | 339 |
| 7 | rs206-c-pre | 2156 | 2194 | | |
| 8 | rs206-t-pre | 2156 | 2194 | | |
| 9 | rs715-t | 12021 | 12059 | 3079 | 3117 |
| 10 | rs715-c | 12021 | 12059 | 3079 | 3117 |

The oligonucleotide of the invention comprises a contiguous nucleotide sequence which is complementary to or hybridizes to the target nucleic acid, such as a target sequence described herein, such as a sequence selected from the group consisting of SEQ ID NO 1-10.

The oligonucleotide of the invention comprises a contiguous nucleotide sequence which is complementary to or hybridizes to the target nucleic acid, such as a target sequence described herein, such as a sequence selected from the group consisting of SEQ ID NOs 3 and 4.

The oligonucleotide of the invention comprises a contiguous nucleotide sequence which is complementary to or hybridizes to the target nucleic acid, such as a target sequence described herein, such as a sequence selected from the group consisting of SEQ ID NOs 5 and 6.

The oligonucleotide of the invention comprises a contiguous nucleotide sequence which is complementary to or hybridizes to the target nucleic acid, such as a target sequence described herein, such as a sequence selected from the group consisting of SEQ ID NOs 7-10.

The target sequence to which the oligonucleotide is complementary or hybridizes to generally comprises a contiguous nucleobases sequence of at least 10 nucleotides. The contiguous nucleotide sequence is between 10 to 40 nucleotides, such as 12 to 30, such as 14 to 20, such as 15 to 18 contiguous nucleotides.

Target Cell

The term a “target cell” as used herein refers to a cell which is expressing the target nucleic acid. In some embodiments the target cell may be in vivo or in vitro. In some embodiments the target cell is a mammalian cell such as a human cell. For experimental purposes, the target call may be an animal cell such as a mouse cell which is heterologously expressing the target nucleic acid.

In preferred embodiments the target cell expresses the target nucleic acid MYH7 mRNA, such as the MYH7 pre-mRNA or MYH7 mature mRNA. The poly A tail of MYH7 mRNA is typically disregarded for antisense oligonucleotide targeting.

Naturally Occurring Variant

The term “naturally occurring variant” refers to variants of MYH7 gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Based on the presence of the sufficient complementary sequence to the oligonucleotide, the oligonucleotide of the invention may therefore target the target nucleic acid and naturally occurring variants thereof.

In some embodiments, the naturally occurring variants have at least 95% such as at least 98% or at least 99% homology or 100% homologous to a mammalian MYH7 target nucleic acid, such as a target nucleic acid selected form the group consisting of SEQ ID NO 1 or SEQ ID NO 2, or a target nucleic acid sequence selected from the group consisting of SEQ ID No 3-10. In some embodiments the naturally occurring variants have at least 99% homology to the human MYH7 target nucleic acid of SEQ ID NO: 1. In some embodiments the naturally occurring variants are the polymorphisms listed in table 3 or 4.

Selectivity

In some aspects it is advantageous that the compounds of the invention have a higher or lower potency against the expression of one allelic variant of MYH7 as compared to the wildtype MYH7 (e.g. SEQ ID NO 1 or 2), for example the allelic variants listed in table 3.

In some aspects it is advantageous that the compounds of the invention have a higher or lower potency against the expression of one allelic variant of MYH7 rs206T as compared to the wildtype MYH7 rs206C.

In some aspects it is advantageous that the compounds of the invention have a higher or lower potency against the expression of one allelic variant of MYH7 rs223C as compared to the wildtype MYH7 rs223T.

In some aspects it is advantageous that the compounds of the invention have a higher or lower potency against the expression of one allelic variant of MYH7 rs715C as compared to the wildtype MYH7 rs715T.

As illustrated in the examples selective inhibition may be determined in vitro in cell lines which are expressing both MYH7 alleles, or in separate cell lines which are each expressing one of the MYH7 allele variants.

Modulation of Expression

The term “modulation of expression” as used herein is to be understood as an overall term for an oligonucleotide's ability to alter the amount of MYH7 when compared to the amount of MYH7 before administration of the oligonucleotide. Alternatively modulation of expression may be determined by reference to a control experiment. It is generally understood that the control is an individual or target cell treated with a saline composition or an individual or target cell treated with a non-targeting oligonucleotide (mock). It may however also be an individual treated with the standard of care.

One type of modulation is the ability of an oligonucleotide to inhibit, down-regulate, reduce, suppress, remove, stop, block, prevent, lessen, lower, avoid or terminate expression of MYH7, e.g. by degradation of mRNA or blockage of transcription.

High Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

Sugar Modifications

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

2′ Sugar Modified Nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

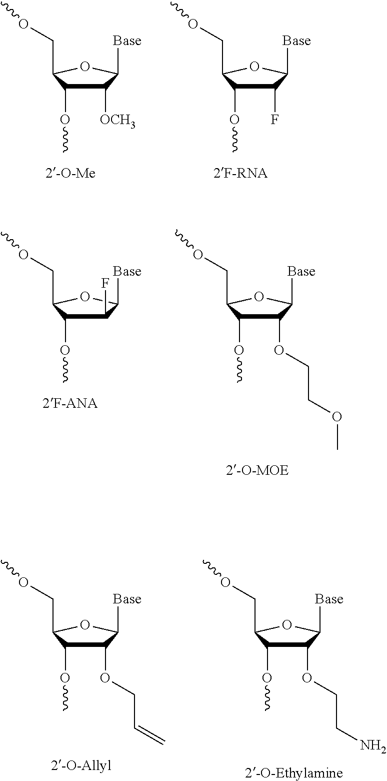

In relation to the present invention 2′ substituted sugar modified nucleosides does not include 2′ bridged nucleosides like LNA.

Locked Nucleic Acids (LNA)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352 , WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667. Further non limiting, exemplary LNA nucleosides are disclosed in Scheme 1.

Scheme 1:

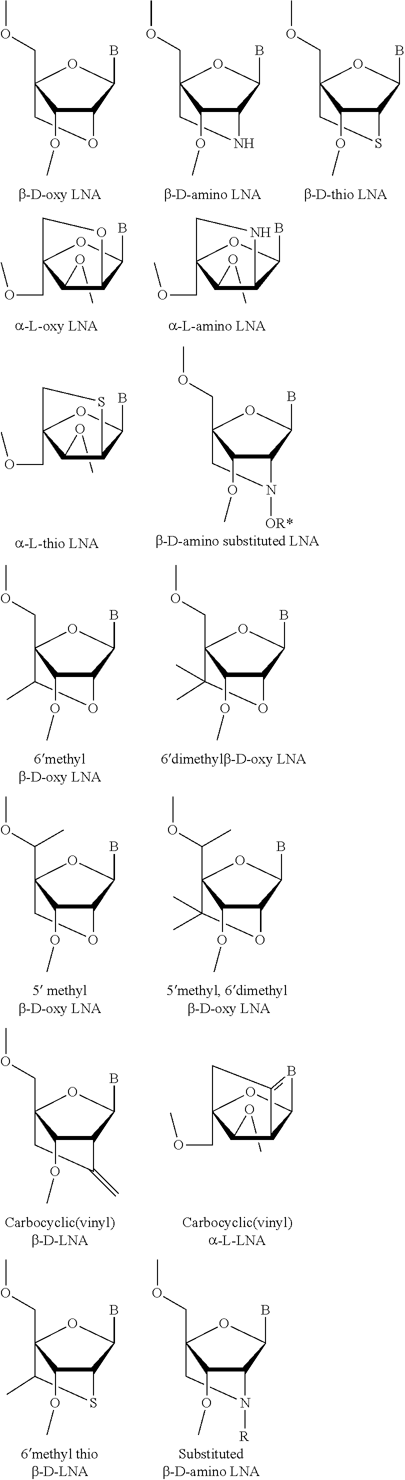

Particular LNA nucleosides are beta-D-oxy-LNA, 6′-methyl-beta-D-oxy LNA such as (S)-6′-methyl-beta-D-oxy-LNA (ScET) and ENA.

A particularly advantageous LNA is beta-D-oxy-LNA.

RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference). For use in determining RHase H activity, recombinant human RNase H1 is available from Lubio Science GmbH, Lucerne, Switzerland.

Gapmer

The antisense oligonucleotide of the invention, or contiguous nucleotide sequence thereof may be a gapmer. The antisense gapmers are commonly used to inhibit a target nucleic acid via RNase H mediated degradation. A gapmer oligonucleotide comprises at least three distinct structural regions a 5′-flank, a gap and a 3′-flank, F-G-F′ in the ‘5→3’ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further defined by having at least one sugar modified nucleoside at the end most distant from the gap region, i.e. at the 5′ end of the 5′ flank and at the 3′ end of the 3′ flank. Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, Such as from 14 to17, such as 16 to18 nucleosides. By way of example, the gapmer oligonucleotide of the present invention can be represented by the following formulae:

F₁₋₈-G₅₋₁₆-F′₁₋₈, such as

F₁₋₈-G₇₋₁₆-F′₂₋₈

with the proviso that the overall length of the gapmer regions F-G-F′ is at least 12, such as at least 14 nucleotides in length.

Regions F, G and F′ are further defined below and can be incorporated into the F-G-F′ formula.

Gapmer—Region G

Region G (gap region) of the gapmer is a region of nucleosides which enables the oligonucleotide to recruit RNaseH, such as human RNase H1, typically DNA nucleosides. RNaseH is a cellular enzyme which recognizes the duplex between DNA and RNA, and enzymatically cleaves the RNA molecule. Suitably gapmers may have a gap region (G) of at least 5 or 6 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15 or 16 contiguous DNA nucleosides. One or more cytosine (C) DNA in the gap region may in some instances be methylated (e.g. when a DNA c is followed by a DNA g) such residues are either annotated as 5-methyl-cytosine (^{me}C). In some embodiments the gap region G may consist of 6, 7, 8, 9, 10, 11, 12, 13, 14, 15 or 16 contiguous phosphorothioate linked DNA nucleosides. In some embodiments, all internucleoside linkages in the gap are phosphorothioate linkages. Whilst traditional gapmers have a DNA gap region, there are numerous examples of modified nucleosides which allow for RNaseH recruitment when they are used within the gap region. Modified nucleosides which have been reported as being capable of recruiting RNaseH when included within a gap region include, for example, alpha-L-LNA, C4′ alkylated DNA (as described in PCT/EP2009/050349 and Vester et al., Bioorg. Med. Chem. Lett. 18 (2008) 2296-2300, both incorporated herein by reference), arabinose derived nucleosides like ANA and 2′F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue. The modified nucleosides used in such gapmers may be nucleosides which adopt a 2′ endo (DNA like) structure when introduced into the gap region, i.e. modifications which allow for RNaseH recruitment). In some embodiments the DNA Gap region (G) described herein may optionally contain 1 to 3 sugar modified nucleosides which adopt a 2′ endo (DNA like) structure when introduced into the gap region.

Region G—“Gap-Breaker”

Alternatively, there are numerous reports of the insertion of a modified nucleoside which confers a 3′ endo conformation into the gap region of gapmers, whilst retaining some RNaseH activity. Such gapmers with a gap region comprising one or more 3′endo modified nucleosides are referred to as “gap-breaker” or “gap-disrupted” gapmers, see for example WO2013/022984. Gap-breaker oligonucleotides retain sufficient region of DNA nucleosides within the gap region to allow for RNaseH recruitment. The ability of gapbreaker oligonucleotide design to recruit RNaseH is typically sequence or even compound specific—see Rukov et al. 2015 Nucl. Acids Res. Vol. 43 pp. 8476-8487, which discloses “gapbreaker” oligonucleotides which recruit RNaseH which in some instances provide a more specific cleavage of the target RNA. Modified nucleosides used within the gap region of gap-breaker oligonucleotides may for example be modified nucleosides which confer a 3′endo confirmation, such 2′-O-methyl (OMe) or 2′-O-MOE (MOE) nucleosides, or beta-D LNA nucleosides (the bridge between C2′ and C4′ of the ribose sugar ring of a nucleoside is in the beta conformation), such as beta-D-oxy LNA or ScET nucleosides.

As with gapmers containing region G described above, the gap region of gap-breaker or gap-disrupted gapmers, have a DNA nucleosides at the 5′ end of the gap (adjacent to the 3′ nucleoside of region F), and a DNA nucleoside at the 3′ end of the gap (adjacent to the 5′ nucleoside of region F′). Gapmers which comprise a disrupted gap typically retain a region of at least 3 or 4 contiguous DNA nucleosides at either the 5′ end or 3′ end of the gap region. Exemplary designs for gap-breaker oligonucleotides include

F₁₋₈-[D₃₋₄-E₁-D₃₋₄]-F′₁₋₈

F₁₋₈-[D₁₋₄-E₁-D₃₋₄]-F′₁₋₈

F₁₋₈-[D₃₋₄-E₁-D₁₋₄]-F′₁₋₈

wherein region G is within the brackets [Dₙ-Eᵣ-Dₘ], D is a contiguous sequence of DNA nucleosides, E is a modified nucleoside (the gap-breaker or gap-disrupting nucleoside), and F and F′ are the flanking regions as defined herein, and with the proviso that the overall length of the gapmer regions F-G-F′ is at least 12, such as at least 14 nucleotides in length. In some embodiments, region G of a gap disrupted gapmer comprises at least 6 DNA nucleosides, such as 6, 7, 8, 9, 10, 11, 12, 13, 14, 15 or 16 DNA nucleosides. As described above, the DNA nucleosides may be contiguous or may optionally be interspersed with one or more modified nucleosides, with the proviso that the gap region G is capable of mediating RNaseH recruitment.

Gapmer—Flanking Regions, F and F′

Region F is positioned immediately adjacent to the 5′ DNA nucleoside of region G. The 3′ most nucleoside of region F is a sugar modified nucleoside, such as a high affinity sugar modified nucleoside, for example a 2′ substituted nucleoside, such as a MOE nucleoside, or an LNA nucleoside.

Region F′ is positioned immediately adjacent to the 3′ DNA nucleoside of region G. The 5′ most nucleoside of region F′ is a sugar modified nucleoside, such as a high affinity sugar modified nucleoside, for example a 2′ substituted nucleoside, such as a MOE nucleoside, or an LNA nucleoside.

Region F is 1-8 contiguous nucleotides in length, such as 2-6, such as 3-4 contiguous nucleotides in length. Advantageously the 5′ most nucleoside of region F is a sugar modified nucleoside. In some embodiments the two 5′ most nucleoside of region F are sugar modified nucleoside. In some embodiments the 5′ most nucleoside of region F is an LNA nucleoside. In some embodiments the two 5′ most nucleoside of region F are LNA nucleosides. In some embodiments the two 5′ most nucleoside of region F are 2′ substituted nucleoside nucleosides, such as two 3′ MOE nucleosides. In some embodiments the 5′ most nucleoside of region F is a 2′ substituted nucleoside, such as a MOE nucleoside.

Region F′ is 2-8 contiguous nucleotides in length, such as 3-6, such as 4-5 contiguous nucleotides in length. Advantageously, embodiments the 3′ most nucleoside of region F′ is a sugar modified nucleoside. In some embodiments the two 3′ most nucleoside of region F′ are sugar modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′ substituted nucleoside nucleosides, such as two 3′ MOE nucleosides. In some embodiments the 3′ most nucleoside of region F′ is a 2′ substituted nucleoside, such as a MOE nucleoside.

It should be noted that when the length of region F or F′ is one, it is advantageously an LNA nucleoside.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, region F and F′ independently comprises both LNA and a 2′ substituted modified nucleosides (mixed wing design).

In some embodiments, region F and F′ consists of only one type of sugar modified nucleosides, such as only MOE or only beta-D-oxy LNA or only ScET. Such designs are also termed uniform flanks or uniform gapmer design.

In some embodiments, all the nucleosides of region F or F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides (an alternating flank, see definition of these for more details). In some embodiments, all the modified nucleosides of region F and F′ are beta-D-oxy LNA nucleosides, wherein region F or F′, or F and F′ may optionally comprise DNA nucleosides (an alternating flank, see definition of these for more details).

In some embodiments the 5′ most and the 3′ most nucleosides of region F and F′ are LNA nucleosides, such as beta-D-oxy LNA nucleosides or ScET nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]₁₋₅-[region G]-[LNA]₁₋₅, wherein region G is as defined in the Gapmer region G definition.

MOE Gapmers

A MOE gapmers is a gapmer wherein regions F and F′ consist of MOE nucleosides. In some embodiments the MOE gapmer is of design [MOE]₁₋₈-[Region G]-[MOE]₁₋₈, such as [MOE]₂₋₇-[Region G]₅₋₁₆-[MOE]₂₋₇, such as [MOE]₃₋₆-[Region G]-[MOE]₃₋₆, wherein region G is as defined in the Gapmer definition. MOE gapmers with a 5-10-5 design (MOE-DNA-MOE) have been widely used in the art.

Mixed Wing Gapmer

A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as a MOE nucleosides. In some embodiments wherein at least one of region F and F′, or both region F and F′ comprise at least one LNA nucleoside, the remaining nucleosides of region F and F′ are independently selected from the group consisting of MOE and LNA. In some embodiments wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides. Mixed wing gapmer designs are disclosed in WO2008/049085 and WO2012/109395, both of which are hereby incorporated by reference.

Alternating Flank Gapmers

Oligonucleotides with alternating flanks are LNA gapmer oligonucleotides where at least one of the flanks (F or F′) comprises DNA in addition to the LNA nucleoside(s). In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides.

In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F or F′ region are LNA nucleosides, and there is at least one DNA nucleoside positioned between the 5′ and 3′ most LNA nucleosides of region F or F′ (or both region F and F′).

Region D′ or D″ in an Oligonucleotide

The oligonucleotide of the invention may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively it may be used to provide exonucleoase protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the oligonucleotide and region D′ or D″ constitute a separate part of the oligonucleotide.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single oligonucleotide.

In one embodiment the oligonucleotide of the invention comprises a region D′ and/or D″ in addition to the contiguous nucleotide sequence which constitutes the gapmer. In some embodiments, the oligonucleotide of the present invention can be represented by the following formulae:

F-G-F′; in particular F₁₋₈-G₅₋₁₆-F′₂₋₈

D′-F-G-F′, in particular D′₁₋₃-F₁₋₈-G₅₋₁₆-F′₂₋₈

F-G-F′-D″, in particular F₁₋₈-G₅₋₁₆-F′₂₋₈-D″₁₋₃

D′-F-G-F′-D″, in particular D′₁₋₃-F₁₋₈-G₅₋₁₆-F′₂₋₈-D″₁₋₃

In some embodiments the internucleoside linkage positioned between region D′ and region F is a phosphodiester linkage. In some embodiments the internucleoside linkage positioned between region F′ and region D″ is a phosphodiester linkage.

Conjugate

The term conjugate as used herein refers to an oligonucleotide which is covalently linked to a non-nucleotide moiety (conjugate moiety or region C or third region).

Conjugation of the oligonucleotide of the invention to one or more non-nucleotide moieties may improve the pharmacology of the oligonucleotide, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular the conjugate may target the oligonucleotide to a specific organ, tissue or cell type and thereby enhance the effectiveness of the oligonucleotide in that organ, tissue or cell type. A the same time the conjugate may serve to reduce activity of the oligonucleotide in non-target cell types, tissues or organs, e.g. off target activity or activity in non-target cell types, tissues or organs.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

Linkers

A linkage or linker is a connection between two atoms that links one chemical group or segment of interest to another chemical group or segment of interest via one or more covalent bonds.

Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence or gapmer region F-G-F′ (region A).

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. DNA phosphodiester containing biocleavable linkers are described in more detail in WO 2014/076195 (hereby incorporated by reference)—see also region D′ or D″ herein.

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups. The oligonucleotide conjugates of the present invention can be constructed of the following regional elements A-C, A-B-C, A-B-Y-C, A-Y-B-C or A-Y-C. In some embodiments the linker (region Y) is an amino alkyl, such as a C2-C36 amino alkyl group, including, for example C6 to C12 amino alkyl groups. In a preferred embodiment the linker (region Y) is a C6 amino alkyl group.

Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic.

Pharmaceutically Acceptable Salts

The compound of the invention may be in the form of a pharmaceutically acceptable salt. The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of hydrochloric acid, hydrobromic acid, sulfuric acid, phosphoric acid and methanesulfonic acid.

Protecting Group

The term “protecting group”, alone or in combination, signifies a group which selectively blocks a reactive site in a multifunctional compound such that a chemical reaction can be carried out selectively at another unprotected reactive site. Protecting groups can be removed. Exemplary protecting groups are amino-protecting groups, carboxy-protecting groups or hydroxy-protecting groups.

### DETAILED DESCRIPTION OF THE INVENTION

The Oligonucleotides of the Invention

The invention relates to antisense oligonucleotides capable of inhibiting expression of human myosin heavy chain 7 (Myh7). The invention relates to antisense oligonucleotides which target MYH7. Described herein are antisense oligonucleotides which provide allelic-specific inhibition of polymorphic variants of myosin heavy chain 7 (Myh7). The invention provides oligonucleotides which target the expression of a MYH7 allelic variant selected from a MYH7 allelic variant which comprises a single nucleotide polymorphism at a position selected from rs2239578, rs2069540, and rs7157716. These three common SNPs are found in intron 2, exon 3, and exon 24 of MYH7 pre-mRNA respectively, and are referred to as rs223, rs206, and rs715 herein.

In some embodiments the oligonucleotide of the invention selectively inhibits a MYH7 allelic variant, such as an allelic variant at a position of the human MYH7 transcript selected from rs223, rs206 and rs715.

In some embodiments the oligonucleotide of the invention selectively inhibits a MYH7 allelic variant of the human MYH7 transcript selected from rs223T or rs223C. In some embodiments the oligonucleotide of the invention selectively inhibits the rs223T MYH7 allelic variant of the human MYH7 transcript selected as compared to the rs223C allelic variant. In some embodiments the oligonucleotide of the invention selectively inhibits the rs223C MYH7 allelic variant of the human MYH7 transcript selected as compared to the rs223T allelic variant.

In some embodiments the oligonucleotide of the invention selectively inhibits a MYH7 allelic variant of the human MYH7 transcript selected from rs206C or rs206T. In some embodiments the oligonucleotide of the invention selectively inhibits the rs206T MYH7 allelic variant of the human MYH7 transcript selected as compared to the rs206C allelic variant. In some embodiments the oligonucleotide of the invention selectively inhibits the rs206C MYH7 allelic variant of the human MYH7 transcript selected as compared to the rs206T allelic variant.

In some embodiments the oligonucleotide of the invention selectively inhibits a MYH7 allelic variant of the human MYH7 transcript selected from rs715C or rs715T. In some embodiments the oligonucleotide of the invention selectively inhibits the rs715T MYH7 allelic variant of the human MYH7 transcript selected as compared to the rs715C allelic variant. In some embodiments the oligonucleotide of the invention selectively inhibits the rs715C MYH7 allelic variant of the human MYH7 transcript selected as compared to the rs715T allelic variant.

In some embodiments the oligonucleotides of the invention targets, such as selectively inhibits, a MYH7 allelic variant found within a human MYH7 intron.

The polymorphisms at rs223, rs206, and rs715 are not considered to be disease associated or disease causing, i.e. they are considered to be silent polymorphisms. However, these three polymorphisms have high heterozygosity across broad demographics and designing oligonucleotides to these SNPs enables multiple disease-linked mutations to be targeted with the same antisense compound. Clinically, this approach requires patient haplotyping to determine if the HCM mutation is on the same allele as the SNP being targeted. The results show that ASOs targeting human SNPs can distinguish alleles containing single nucleotide mismatches with both high potency (e.g. <100 nM) and high selectivity (e.g. >20×). This strategy can be applied therapeutically when a patient harbors the pathogenic MYH7 mutation and the SNP of interest on the same transcript.

In some embodiments the antisense oligonucleotide of the invention is capable of modulating the expression of the target by inhibiting or down-regulating it. Preferably, such modulation produces an inhibition of expression of at least 20% compared to the normal expression level of the target, more preferably at least 30%, at least 40%, at least 50%, at least 60%, at least 70%, at least 80%, or at least 90% inhibition compared to the normal expression level of the target. In some embodiments oligonucleotides of the invention may be capable of inhibiting expression levels of MYH7 mRNA by at least 50%, such as at least 60% or at least 70% in vitro using Human iPSC-derived cardiomyocytes (available from Cellular Dynamics International) or human skeletal muscle myoblasts cells, such as 8220 or NH10-637A cells (see the examples for exemplary methodology) .

An aspect of the present invention relates to an antisense oligonucleotide which comprises a contiguous nucleotide sequence of 10 to 30 nucleotides in length with at least 90% complementarity to human MYD7 mature mRNA or pre-mRNA.

In some embodiments, the oligonucleotide comprises a contiguous sequence of 10 to 30 nucleotides in length, which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence, such as a sequence selected from SEQ ID NO 3-10.

In a preferred embodiment the oligonucleotide of the invention, or contiguous nucleotide sequence thereof is fully complementary (100% complementary) to a region of the target nucleic acid, such as a sequence selected from SEQ ID NO 3-10.

In some embodiments the oligonucleotide comprises a contiguous nucleotide sequence of 10 to 30 nucleotides in length with at least 90% complementary, such as fully (or 100%) complementary, to a region target nucleic acid region present in SEQ ID NO: 1 or SEQ ID NO 2.

In some embodiments the oligonucleotide comprises a contiguous nucleotide sequence of 10 to 30 nucleotides in length with at least 90% complementary, such as fully (or 100%) complementary, to a region target nucleic acid region present in SEQ ID NO: 3-10.

In some embodiments, the oligonucleotide of the invention comprises or consists of 10 to 35 nucleotides in length, such as from 10 to 30, such as 11 to 24, such as from 12 to 22, such as from 14 to 20 or 14 to 18 or 15 to 19 contiguous nucleotides in length.

In some embodiments, the oligonucleotide or contiguous nucleotide sequence thereof comprises or consists of 22 or less nucleotides, such as 20 or less nucleotides, such as 19 or less or 18 or less nucleotides, such as 14, 15, 16 or 17 nucleotides. It is to be understood that any range given herein includes the range endpoints. Accordingly, if an oligonucleotide is said to include from 10 to 30 nucleotides, both 10 and 30 nucleotides are included.

In some embodiments, the contiguous nucleotide sequence comprises or consists of 10, 11, 12, 13, 14, 15, 16, 17, 18, 19, 20, 21, 22, 23, 24, 25, 26, 27, 28, 29 or 30 contiguous nucleotides in length.

In some embodiments, the oligonucleotide or contiguous nucleotide sequence comprises or consists of a sequence selected from the group consisting of sequences listed in table 5.

Table 5 provides the compound list of the compounds used in the examples, including reference to the SEQ IDs of the compounds, and the gapmer designs of the LNA compounds. In some embodiments, for the compounds listed in table 5, capital letters=LNA nucleosides, lower case letter=DNA nucleosides, and optionally all internucleoside linkages are phosphorothioate. In some embodiments of the listed gapmer compounds, and as used in the examples, capital letters=beta-D-oxy-LNA nucleosides, LNA cytosines=5 methyl cytosine LNA, lower case letters=DNA nucleosides, and all internucleoside linkages between the nucleosides illustrated are phosphorothioate internucleoside linkages.

In some embodiments, the oligonucleotide of the invention, or contiguous nucleotide sequence thereof, comprises at least 10 contiguous nucleosides present in a sequence selected from the group consisting of SEQ ID NO 11-344. In some embodiments, the oligonucleotide of the invention, or contiguous nucleotide sequence thereof, comprises at least 12 contiguous nucleosides present in a sequence selected from the group consisting of SEQ ID NO 11-344. In some embodiments, the oligonucleotide of the invention, or contiguous nucleotide sequence thereof, comprises at least 14 contiguous nucleosides present in a sequence selected from the group consisting of SEQ ID NO 11-344. In some embodiments, the oligonucleotide of the invention, or contiguous nucleotide sequence thereof, comprises at least 15 or at least 16 contiguous nucleosides present in a sequence selected from the group consisting of SEQ ID NO 11-344. In some embodiments, the oligonucleotide of the invention, or contiguous nucleotide sequence thereof, comprises a sequence selected from the group consisting of SEQ ID NO 11-344.

Oligonucleotides targeting the rs206c myh7 allele:

In some embodiments, the oligonucleotide of the invention, or contiguous nucleotide sequence thereof, comprises at least 10 contiguous nucleosides present in a sequence selected from the group consisting of SEQ ID NO 11-85. In some embodiments, the oligonucleotide of the invention, or contiguous nucleotide sequence thereof, comprises at least 12 contiguous nucleosides present in a sequence selected from the group consisting of SEQ ID NO 11-85. In some embodiments, the oligonucleotide of the invention, or contiguous nucleotide sequence thereof, comprises at least 14 contiguous nucleosides present in a sequence selected from the group consisting of SEQ ID NO 11-85. In some embodiments, the oligonucleotide of the invention, or contiguous nucleotide sequence thereof, comprises at least 16 contiguous nucleosides present in a sequence selected from the group consisting of SEQ ID NO 11-85. In some embodiments, the oligonucleotide of the invention, or contiguous nucleotide sequence thereof, comprises a sequence selected from the group consisting of SEQ ID NO 11-85.

Oligonucleotides targeting the rs206t myh7 allele:

In some embodiments, the oligonucleotide of the invention, or contiguous nucleotide sequence thereof, comprises at least 10 contiguous nucleosides present in a sequence selected from the group consisting of SEQ ID NO 86-140. In some embodiments, the oligonucleotide of the invention, or contiguous nucleotide sequence thereof, comprises at least 12 contiguous nucleosides present in a sequence selected from the group consisting of SEQ ID NO 86-140. In some embodiments, the oligonucleotide of the invention, or contiguous nucleotide sequence thereof, comprises at least 14 contiguous nucleosides present in a sequence selected from the group consisting of SEQ ID NO 86-140. In some embodiments, the oligonucleotide of the invention, or contiguous nucleotide sequence thereof, comprises at least 16 contiguous nucleosides present in a sequence selected from the group consisting of SEQ ID NO 86-140. In some embodiments, the oligonucleotide of the invention, or contiguous nucleotide sequence thereof, comprises a sequence selected from the group consisting of SEQ ID NO 86-140.

Oligonucleotides targeting the rs223c myh7 allele:

In some embodiments, the oligonucleotide of the invention, or contiguous nucleotide sequence thereof, comprises at least 10 contiguous nucleosides present in a sequence selected from the group consisting of SEQ ID NO 141-192. In some embodiments, the oligonucleotide of the invention, or contiguous nucleotide sequence thereof, comprises at least 12 contiguous nucleosides present in a sequence selected from the group consisting of SEQ ID NO 141-192. In some embodiments, the oligonucleotide of the invention, or contiguous nucleotide sequence thereof, comprises at least 14 contiguous nucleosides present in a sequence selected from the group consisting of SEQ ID NO 141-192. In some embodiments, the oligonucleotide of the invention, or contiguous nucleotide sequence thereof, comprises at least 16 contiguous nucleosides present in a sequence selected from the group consisting of SEQ ID NO 141-192. In some embodiments, the oligonucleotide of the invention, or contiguous nucleotide sequence thereof, comprises a sequence selected from the group consisting of SEQ ID NO 141-192.

Oligonucelotides targeting the rs233t myh7 allele:

In some embodiments, the oligonucleotide of the invention, or contiguous nucleotide sequence thereof, comprises at least 10 contiguous nucleosides present in a sequence selected from the group consisting of SEQ ID NO 193-251. In some embodiments, the oligonucleotide of the invention, or contiguous nucleotide sequence thereof, comprises at least 12 contiguous nucleosides present in a sequence selected from the group consisting of SEQ ID NO 193-251. In some embodiments, the oligonucleotide of the invention, or contiguous nucleotide sequence thereof, comprises at least 14 contiguous nucleosides present in a sequence selected from the group consisting of SEQ ID NO 193-251. In some embodiments, the oligonucleotide of the invention, or contiguous nucleotide sequence thereof, comprises at least 16 contiguous nucleosides present in a sequence selected from the group consisting of SEQ ID NO 193-251. In some embodiments, the oligonucleotide of the invention, or contiguous nucleotide sequence thereof, comprises a sequence selected from the group consisting of SEQ ID NO 193-251.

Oligonucleotide targeting the rs715c myh7 allele:

In some embodiments, the oligonucleotide of the invention, or contiguous nucleotide sequence thereof, comprises at least 10 contiguous nucleosides present in a sequence selected from the group consisting of SEQ ID NO 252-266. In some embodiments, the oligonucleotide of the invention, or contiguous nucleotide sequence thereof, comprises at least 12 contiguous nucleosides present in a sequence selected from the group consisting of SEQ ID NO 252-266. In some embodiments, the oligonucleotide of the invention, or contiguous nucleotide sequence thereof, comprises at least 14 contiguous nucleosides present in a sequence selected from the group consisting of SEQ ID NO 252-266. In some embodiments, the oligonucleotide of the invention, or contiguous nucleotide sequence thereof, comprises at least 16 contiguous nucleosides present in a sequence selected from the group consisting of SEQ ID NO 252-266. In some embodiments, the oligonucleotide of the invention, or contiguous nucleotide sequence thereof, comprises a sequence selected from the group consisting of SEQ ID NO 252-266.

Oligonucleotide targeting the rs715t myh7 allele:

In some embodiments, the oligonucleotide of the invention, or contiguous nucleotide sequence thereof, comprises at least 10 contiguous nucleosides present in a sequence selected from the group consisting of SEQ ID NO 267-298. In some embodiments, the oligonucleotide of the invention, or contiguous nucleotide sequence thereof, comprises at least 12 contiguous nucleosides present in a sequence selected from the group consisting of SEQ ID NO 267-298. In some embodiments, the oligonucleotide of the invention, or contiguous nucleotide sequence thereof, comprises at least 14 contiguous nucleosides present in a sequence selected from the group consisting of SEQ ID NO 267-298. In some embodiments, the oligonucleotide of the invention, or contiguous nucleotide sequence thereof, comprises at least 16 contiguous nucleosides present in a sequence selected from the group consisting of SEQ ID NO 267-298. In some embodiments, the oligonucleotide of the invention, or contiguous nucleotide sequence thereof, comprises a sequence selected from the group consisting of SEQ ID NO 267-298.

Other oligonucleotides targeting human Myh7:

In some embodiments, the oligonucleotide of the invention, or contiguous nucleotide sequence thereof, comprises at least 10 contiguous nucleosides present in a sequence selected from the group consisting of SEQ ID NO 299-344. In some embodiments, the oligonucleotide of the invention, or contiguous nucleotide sequence thereof, comprises at least 12 contiguous nucleosides present in a sequence selected from the group consisting of SEQ ID NO 299-344. In some embodiments, the oligonucleotide of the invention, or contiguous nucleotide sequence thereof, comprises at least 14 contiguous nucleosides present in a sequence selected from the group consisting of SEQ ID NO 299-344. In some embodiments, the oligonucleotide of the invention, or contiguous nucleotide sequence thereof, comprises at least 16 contiguous nucleosides present in a sequence selected from the group consisting of SEQ ID NO 299-344.

In some embodiments, the oligonucleotide of the invention, or contiguous nucleotide sequence thereof, comprises a sequence selected from the group consisting of SEQ ID NO 299-344.

In some embodiments, the oligonucleotide of the invention at least 70% of the internucleoside linkages are phosphorothioate, such as at least 90% of the internucleoside linkages are phosphorothioate. In some embodiments, all the internucleoside linkages between the nucleosides of the contiguous nucleotide sequence of the oligonucleotide of the invention are phosphorothioate internucleoside linkages.

It is understood that the contiguous nucleobase sequences (motif sequence) can be modified to for example increase nuclease resistance and/or binding affinity to the target nucleic acid.

The pattern in which the modified nucleosides (such as high affinity modified nucleosides) are incorporated into the oligonucleotide sequence is generally termed oligonucleotide design.

In some embodiments, the oligonucleotides of the invention are designed with modified nucleosides and DNA nucleosides. Advantageously, high affinity modified nucleosides are used.

In an embodiment, the oligonucleotide or contiguous nuceltoide sequence thereof, comprises at least 1 modified nucleoside, such as at least 2, at least 3, at least 4, at least 5, at least 6, at least 7, at least 8, at least 9, at least 10, at least 11, at least 12, at least 13, at least 14, at least 15 or at least 16 modified nucleosides. In an embodiment the oligonucleotide comprises from 1 to 10 modified nucleosides, such as from 2 to 9 modified nucleosides, such as from 3 to 8 modified nucleosides, such as from 4 to 7 modified nucleosides, such as 6 or 7 modified nucleosides. Suitable modifications are described in the “Definitions” section under “modified nucleoside”, “high affinity modified nucleosides”, “sugar modifications”, “2′ sugar modifications” and Locked nucleic acids (LNA)”.

In an embodiment, the oligonucleotide or contiguous nucleotide sequence thereof, comprises one or more sugar modified nucleosides, such as 2′ sugar modified nucleosides. Preferably the oligonucleotide of the invention comprise one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

In a further embodiment the oligonucleotide comprises at least one modified internucleoside linkage. Suitable internucleoside modifications are described in the “Definitions” section under “Modified internucleoside linkage”. It is advantageous if at least 75%, such as all, the internucleoside linkages within the contiguous nucleotide sequence are phosphorothioate or boranophosphate internucleoside linkages. In some embodiments all the internucleotide linkages in the contiguous sequence of the oligonucleotide are phosphorothioate linkages.

In some embodiments, the oligonucleotide, or contiguous nuceltoide sequence thereof, of the invention comprises at least one LNA nucleoside, such as 1, 2, 3, 4, 5, 6, 7, or 8 LNA nucleosides, such as from 2 to 6 LNA nucleosides, such as from 3 to 7 LNA nucleosides, 4 to 8 LNA nucleosides or 3, 4, 5, 6, 7 or 8 LNA nucleosides. In some embodiments, at least 75% of the modified nucleosides in the oligonucleotide are LNA nucleosides, such as 80%, such as 85%, such as 90% of the modified nucleosides are LNA nucleosides. In a still further embodiment all the modified nucleosides in the oligonucleotide are LNA nucleosides. In a further embodiment, the oligonucleotide may comprise both beta-D-oxy-LNA, and one or more of the following LNA nucleosides: thio-LNA, amino-LNA, oxy-LNA, ScET and/or ENA in either the beta-D or alpha-L configurations or combinations thereof. In a further embodiment, all LNA cytosine units are 5-methyl-cytosine. It is advantageous for the nuclease stability of the oligonucleotide or contiguous nucleotide sequence to have at least 1 LNA nucleoside at the 5′ end and at least 2 LNA nucleosides at the 3′ end of the nucleotide sequence.

In some embodiments of the invention the oligonucleotide of the invention is capable of recruiting RNase H.

In the current invention an advantageous structural design is a gapmer design as described in the “Definitions” section under for example “Gapmer”, “LNA Gapmer”, “MOE gapmer” and “Mixed Wing Gapmer” “Alternating Flank Gapmer”. The gapmer design includes gapmers with uniform flanks, mixed wing flanks, alternating flanks, and gapbreaker designs. In the present invention it is advantageous if the oligonucleotide of the invention is a gapmer with an F-G-F′ design.

For some embodiments of the invention, the oligonucleotide or contiguous nucleotide sequence thereof, is selected from the group of oligonucleotide compounds with CMP-ID-NO (COMP #) 11-85.

For some embodiments of the invention, the oligonucleotide or contiguous nucleotide sequence thereof, is selected from the group of oligonucleotide compounds with CMP-ID-NO (COMP #) 86-140.

For some embodiments of the invention, the oligonucleotide or contiguous nucleotide sequence thereof, is selected from the group of oligonucleotide compounds with CMP-ID-NO (COMP #) 141-192.

For some embodiments of the invention, the oligonucleotide or contiguous nucleotide sequence thereof, is selected from the group of oligonucleotide compounds with CMP-ID-NO (COMP #) 193-251.

For some embodiments of the invention, the oligonucleotide or contiguous nucleotide sequence thereof, is selected from the group of oligonucleotide compounds with CMP-ID-NO (COMP #) 252-266.

For some embodiments of the invention, the oligonucleotide or contiguous nucleotide sequence thereof, is selected from the group of oligonucleotide compounds with CMP-ID-NO (COMP #) 259,1-259,189.

For some embodiments of the invention, the oligonucleotide or contiguous nucleotide sequence thereof, is selected from the group of oligonucleotide compounds with CMP-ID-NO (COMP #) 260,1-260,101.

For some embodiments of the invention, the oligonucleotide or contiguous nucleotide sequence thereof, is selected from the group of oligonucleotide compounds with CMP-ID-NO (COMP #) 267-298.

For some embodiments of the invention, the oligonucleotide or contiguous nucleotide sequence thereof, is selected from the group of oligonucleotide compounds with CMP-ID-NO (COMP #) 280,1-280,161.

For some embodiments of the invention, the oligonucleotide or contiguous nucleotide sequence thereof, is selected from the group of oligonucleotide compounds with CMP-ID-NO (COMP #) 299-344.

Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand) to covalently attach the conjugate moiety to the oligonucleotide. In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant.

Pharmaceutical Salt

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin, Organic Process Research & Development 2000, 4, 427-435 or in Ansel, In: Pharmaceutical Dosage Forms and Drug Delivery Systems, 6th ed. (1995), pp. 196 and 1456-1457. For example, the pharmaceutically acceptable salt of the compounds provided herein may be a sodium salt.

In a further aspect the invention provides a pharmaceutically acceptable salt of the antisense oligonucleotide or a conjugate thereof. In a preferred embodiment, the pharmaceutically acceptable salt is a sodium or a potassium salt.

Pharmaceutical Composition

In a further aspect, the invention provides pharmaceutical compositions comprising any of the aforementioned oligonucleotides and/or oligonucleotide conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50-300 μM solution.

Suitable formulations for use in the present invention are found in Remington's Pharmaceutical Sciences, Mack Publishing Company, Philadelphia, Pa., 17th ed., 1985. Fora brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

Oligonucleotides or oligonucleotide conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the oligonucleotide or oligonucleotide conjugate of the invention is a prodrug. In particular with respect to oligonucleotide conjugates the conjugate moiety is cleaved of the oligonucleotide once the prodrug is delivered to the site of action, e.g. the target cell.

Applications

The oligonucleotides of the invention may be utilized as research reagents for, for example, diagnostics, therapeutics and prophylaxis.

In research, such oligonucleotides may be used to specifically modulate the synthesis of MYH7 protein in cells (e.g. in vitro cell cultures) and experimental animals thereby facilitating functional analysis of the target or an appraisal of its usefulness as a target for therapeutic intervention. Typically the target modulation is achieved by degrading or inhibiting the mRNA producing the protein, thereby prevent protein formation or by degrading or inhibiting a modulator of the gene or mRNA producing the protein.

If employing the oligonucleotide of the invention in research or diagnostics the target nucleic acid may be a cDNA or a synthetic nucleic acid derived from DNA or RNA.

The present invention provides an in vivo or in vitro method for modulating MYH7 expression in a target cell which is expressing MYH7, said method comprising administering an oligonucleotide of the invention in an effective amount to said cell.

In some embodiments, the target cell, is a mammalian cell in particular a human cell. The target cell may be an in vitro cell culture or an in vivo cell forming part of a tissue in a mammal. In preferred embodiments the target cell is a muscle cell, a skeletal muscle cell, a heart cell, or a cardiomyocyte cell.

In diagnostics the oligonucleotides may be used to detect and quantitate MYH7 expression in cell and tissues by northern blotting, in-situ hybridisation or similar techniques.

For therapeutics, the oligonucleotides may be administered to an animal or a human, suspected of having a disease or disorder, which can be treated by modulating the expression of MYH7.

The invention provides methods for treating or preventing a disease, comprising administering a therapeutically or prophylactically effective amount of an oligonucleotide, an oligonucleotide conjugate or a pharmaceutical composition of the invention to a subject suffering from or susceptible to the disease.

The invention also relates to an oligonucleotide, a composition or a conjugate as defined herein for use as a medicament.

The oligonucleotide, oligonucleotide conjugate or a pharmaceutical composition according to the invention is typically administered in an effective amount.

The invention also provides for the use of the oligonucleotide or oligonucleotide conjugate of the invention as described for the manufacture of a medicament for the treatment of a disorder as referred to herein, or for a method of the treatment of as a disorder as referred to herein.

The disease or disorder, as referred to herein, is associated with expression of MYH7. In some embodiments disease or disorder may be associated with a mutation in the MYH7 gene or a gene whose protein product is associated with or interacts with MYH7. Therefore, in some embodiments, the target nucleic acid is a mutated form of the MYH7 sequence and in other embodiments, the target nucleic acid is a regulator of the MYH7 sequence.

The methods of the invention are preferably employed for treatment or prophylaxis against diseases caused by abnormal levels and/or activity of MYH7.

The invention further relates to use of an oligonucleotide, oligonucleotide conjugate or a pharmaceutical composition as defined herein for the manufacture of a medicament for the treatment of abnormal levels and/or activity of MYH7.

In one embodiment, the invention relates to oligonucleotides, oligonucleotide conjugates or pharmaceutical compositions for use in the treatment of

Administration

The oligonucleotides or pharmaceutical compositions of the present invention may be administered topical (such as, to the skin, inhalation, ophthalmic or otic) or enteral (such as, orally or through the gastrointestinal tract) or parenteral (such as, intravenous, subcutaneous, intra-muscular, intracerebral, intracerebroventricular or intrathecal).

In a preferred embodiment the oligonucleotide or pharmaceutical compositions of the present invention are administered by a parenteral route including intravenous, intraarterial, subcutaneous, intraperitoneal or intramuscular injection or infusion, intrathecal or intracranial, e.g. intracerebral or intraventricular, intravitreal administration. In one embodiment the active oligonucleotide or oligonucleotide conjugate is administered intravenously. In another embodiment the active oligonucleotide or oligonucleotide conjugate is administered subcutaneously.

In some embodiments, the oligonucleotide, oligonucleotide conjugate or pharmaceutical composition of the invention is administered at a dose of 0.1-15 mg/kg, such as from 0.2-10 mg/kg, such as from 0.25-5 mg/kg. The administration can be once a week, every 2^{nd} week, every third week or even once a month.

The invention also provides for the use of the oligonucleotide or oligonucleotide conjugate of the invention as described for the manufacture of a medicament wherein the medicament is in a dosage form for intravenous or subcutaneous administration.

Combination Therapies

In some embodiments the oligonucleotide, oligonucleotide conjugate or pharmaceutical composition of the invention is for use in a combination treatment with another therapeutic agent. The therapeutic agent can for example be the standard of care for the diseases or disorders described above.

Personalized Method of Treatment Using Allelic Specific Compounds Targeting Myh7

The invention provides for a method for treatment of a human subject in need to treatment for hypertrophic cardiomyopathy, said treatment comprising the step of:

a. Taking a biological sample from the human subject

b. Detecting such as sequencing the Myh7 nucleic acid alleles present in the sample of the human subject;

c. Determine the presence of a disease associated Myh7 allelic variant of the Myh7 nucleic acid;

d. Administer a therapeutically effective amount of an antisense oligonucleotide to the human subject which is selective for the disease associated Myh7 allelic variant as compared to a non-disease associate allele, such as the oligonucleotide of the invention or the conjugate of the invention or the pharmaceutically acceptable salt of the invention or the pharmaceutical composition of the invention.

### EXAMPLES

Materials and Methods

Oligonucleotide Synthesis

Oligonucleotide synthesis is generally known in the art. Below is a protocol which may be applied. The oligonucleotides of the present invention may have been produced by slightly varying methods in terms of apparatus, support and concentrations used.

Oligonucleotides are synthesized on uridine universal supports using the phosphoramidite approach on an Oligomaker 48 at 1 μmol scale. At the end of the synthesis, the oligonucleotides are cleaved from the solid support using aqueous ammonia for 5-16 hours at 60° C. The oligonucleotides are purified by reverse phase HPLC (RP-HPLC) or by solid phase extractions and characterized by UPLC, and the molecular mass is further confirmed by ESI-MS.

Elongation of the Oligonucleotide:

The coupling of β-cyanoethyl-phosphoramidites (DNA-A(Bz), DNA-G(ibu), DNA-C(Bz), DNA-T, LNA-5-methyl-C(Bz), LNA-A(Bz), LNA-G(dmf), or LNA-T) is performed by using a solution of 0.1 M of the 5′-O-DMT-protected amidite in acetonitrile and DCI (4,5-dicyanoimidazole) in acetonitrile (0.25 M) as activator. For the final cycle a phosphoramidite with desired modifications can be used, e.g. a C6 linker for attaching a conjugate group or a conjugate group as such. Thiolation for introduction of phosphorthioate linkages is carried out by using xanthane hydride (0.01 M in acetonitrile/pyridine 9:1). Phosphordiester linkages can be introduced using 0.02 M iodine in THF/Pyridine/water 7:2:1. The rest of the reagents are the ones typically used for oligonucleotide synthesis.

For post solid phase synthesis conjugation a commercially available C6 aminolinker phorphoramidite can be used in the last cycle of the solid phase synthesis and after deprotection and cleavage from the solid support the aminolinked deprotected oligonucleotide is isolated. The conjugates are introduced via activation of the functional group using standard synthesis methods.

Purification by RP-HPLC:

The crude compounds are purified by preparative RP-HPLC on a Phenomenex Jupiter C18 10μ 150×10 mm column. 0.1 M ammonium acetate pH 8 and acetonitrile is used as buffers at a flow rate of 5 mL/min. The collected fractions are lyophilized to give the purified compound typically as a white solid.

Abbreviations:

DCI: 4,5-Dicyanoimidazole

DCM: Dichloromethane

DMF: Dimethylformamide

DMT: 4,4′-Dimethoxytrityl

THF: Tetrahydrofurane

Bz: Benzoyl

Ibu: Isobutyryl

RP-HPLC: Reverse phase high performance liquid chromatography

Tₘ Assay:

Oligonucleotide and RNA target (phosphate linked, PO) duplexes are diluted to 3 mM in 500 ml RNase-free water and mixed with 500 ml 2× Tm-buffer (200 mM NaCl, 0.2 mM EDTA, 20 mM Naphosphate, pH 7.0). The solution is heated to 95° C. for 3 min and then allowed to anneal in room temperature for 30 min. The duplex melting temperatures (Tₘ) is measured on a Lambda 40 UV/VIS Spectrophotometer equipped with a Peltier temperature programmer PTP6 using PE Templab software (Perkin Elmer). The temperature is ramped up from 20° C. to 95° C. and then down to 25° C., recording absorption at 260 nm. First derivative and the local maximums of both the melting and annealing are used to assess the duplex Tₘ.

ASO Synthesis and Purification

LNA-modified gapmers were designed with fully modified phosphorothioate backbones and were synthesized on a MerMade 192× synthesizer (Bioautomation, Texas) following standard phosphoramidite protocols. The final 5′-dimethoxytrityl (DMT) group was left on the oligonucleotide. After synthesis, the oligonucleotides were cleaved from the solid support using aqueous ammonia and subsequently deprotected at 65° C. for 5 hours. The oligonucleotides were purified by solid phase extraction in TOP DNA cartridges (Agilent, Glostrup, Denmark) using the lipophilic DMT group as a chromatographic retention probe. After eluting impurities, the DMT group was removed by treatment with dichloroacetic acid. As the last step in the purification process, the oligonucleotides were eluted from the cartridge and the eluate was evaporated to dryness. The oligonucleotides were dissolved in phosphate-buffered saline (PBS) and the oligonucleotide concentration in solution determined using Beer-Lambert's law by calculating the extinction coefficient and measuring UV-absorbance. Oligonucleotide identity and purity were determined by reversed-phase Ultra Performance Liquid Chromatography coupled to Mass Spectrometry (UPLC-MS).

Cell Culture

Human skeletal muscle myoblasts (8220 and NH10-637A [9]) were seeded in collagen-coated 96 well plates at a density of 15,000 cells/well. Cells were maintained in SKM-M growth media (ZenBio, North Carolina) until confluence, at which point SKM-D differentiation media was used. Cells were cultured for 1 week in differentiation media to allow for myoblast fusion and differentiation into myotubes, with media exchange every other day. One week after switching to differentiation media, ASOs were added to the cells in the absence of transfection reagents (i.e. gymnotic delivery); biological duplicates were used. Cells were lysed at day 3 or day 6 for single point studies and at day 6 or day 10 for concentration response curves. Human iPSC-derived cardiomyocytes were purchased from Cellular Dynamics International and cultured according to the manufacturer's instructions. Cells were seeded in collagen/fibronectin coated (0.01 mg/ml) 96 well plates at a density of 20,000 cells per well. ASOs dissolved in PBS or water were added 4 days after plating and media was changed every other day until lysis.

QuantiGene

The QuantiGene 2.0 assay (Affymetrix) was used to quantify RNA abundance of MYH7 (QG probe SA-10161) and the endogenous control (Human PPIB probe SA-10003) of each lysate following the manufacturer's protocol. The QG probes are designed to exonic regions of MYH7 and PPIB. Assay signals were background subtracted and normalized to the endogenous control to correct for cell density and lysis efficiency. MYH7 knockdown is reported relative to no ASO negative control.

RNA Purification and ddPCR

Cells were lysed by removal of media followed by addition of 125 μL PureLink©Pro 96 Lysis buffer (Invitrogen 12173.001A) and 125 μL 70% ethanol. RNA was purified according to the manufacture's instruction and eluted in a final volume of 50 μL water resulting in an RNA concentration of 10-20 ng/μl. Droplet digital PCR (ddPCR) was done using BioRad Automatic Droplet Generator (AutoDG) using Automated Droplet Generation Oil for Probes (BioRad) together with the OX200 droplet digital reader. The ddPCR™ Supermix for Probes (No dUTP) (Bio-Rad 1863024) reactions were run according to the manufacturer's instructions with an annealing temperature of 55.5° C. for the human reactions and 55° C. for the mouse reactions. The droplets were read in the OX200 droplet digital reader, and the data were analyzed and quantified using the QuantaSoft™ Analysis Pro Software 1.0.596 (BioRad). The thresholds for defining the different droplet groups in the triplex PCR reaction was set by free hand within the software according to the guidelines. Assays for human SNPs: rs715T (fw_primer CAGAGGAGATGGCTGG, rev_primer TGCAGAGCTTTCTTCTCC (SEQ ID NO 345), probe CAGCTTGGCAATGATCTC HEX_IowaBlack, (SEQ ID NO 346)); rs715C (fw_primer CAGAGGAGATGGCTGG (SEQ ID NO 347), rev_primer TGCAGAGCTTTCTTCTCC (SEQ ID NO 348), probe CAGCTTGGCGATGATCT FAM_IowaBlack (SEQ ID NO 349)); GAPDH (dHsa CPE5031596, FAM_IowaBlack) and (dHsa CPE5031597, HEX_IowaBlack) from BioRad. Assays for humanized mouse model: humanized rs715C myh6 (fw_primer CCTAACAGAGGAGATG (SEQ ID NO 350), rev_primer CTTCTTGCAGAGCTTTCTT (SEQ ID NO 351), probe TGAGATCATCGCCAAGC Hex_IowaBlack (SEQ ID NO 352)); wt myh6 (fw_primer ACCTAACAGAGGAGATG (SEQ ID NO 353), rev_primer CTTCTTGCAGAGCTTTCTT (SEQ ID NO 354), probe TGAAATCATTGCCAAGCTG FAM_IowaBlack (SEQ ID NO 355)); GAPDH (dMmuCPE5195282, FAM_IowaBlack and dMmuCPE5195283, HEX_IowaBlack) from BioRad.

Statistical Analysis of Concentration-Response Curves

Concentration-response curves of RNA levels after treatment with ASO at eight different concentrations were analyzed by nonlinear least squares fitting of the two-parameter logistic function using the R software package drc [10]. For the two-parameter logistic function the lower and upper limits are fixed at 0% and 100%, respectively, and the two parameters estimated from each curve are the IC50 value and Hill coefficient. The maximal possible IC₅₀ value was set to the maximal ASO concentration evaluated.

Mouse Model Generation and In Vivo Study

Since the predominant isoform in mouse heart is Myh6, the human MYH7 sequence (ENSG00000092054) around the rs715-C SNP was inserted into the mouse Myh6 gene (ENSMUSG00000040752) using homologous recombination in C57BL/6J mice (Figure S4). This 57 nucleotide insertion (acagaggagatggctgggctggatgagatcatCgccaagctgaccaaggagaagaaa (SEQ ID NO 356) replacing acagaggagatggctgggctggatgaaatcatTgccaagctgaccaaagagaagaaa, SEQ ID NO 357) is not predicted to affect amino acid sequence (SNP nucleotide shown as uppercase). Mice are homozygous for thymine at the base position that corresponds to the rs715 SNP in humans. Heterozygous mice (human rs715-C)^{+/-} lacking FLP recombinase were used for the in vivo study. Animals were dosed with ASO subcutaneously at 3*30 mg/kg on days 0, 1, and 2 with takedown on day 7. Allele-specific Myh6 mRNA knockdown was measured via droplet digital PCR from RNA isolated from half of the left ventricle. The other half of the left ventricle, in addition to one kidney and a portion of liver, was quick frozen in liquid nitrogen to determine the tissue concentrations of ASO (Oligo ELISA, Exiqon, Denmark). Blood was collected at the time of sacrifice, with subsequent serum quantification of kidney and liver injury markers.

### Example 1: SNP Identification

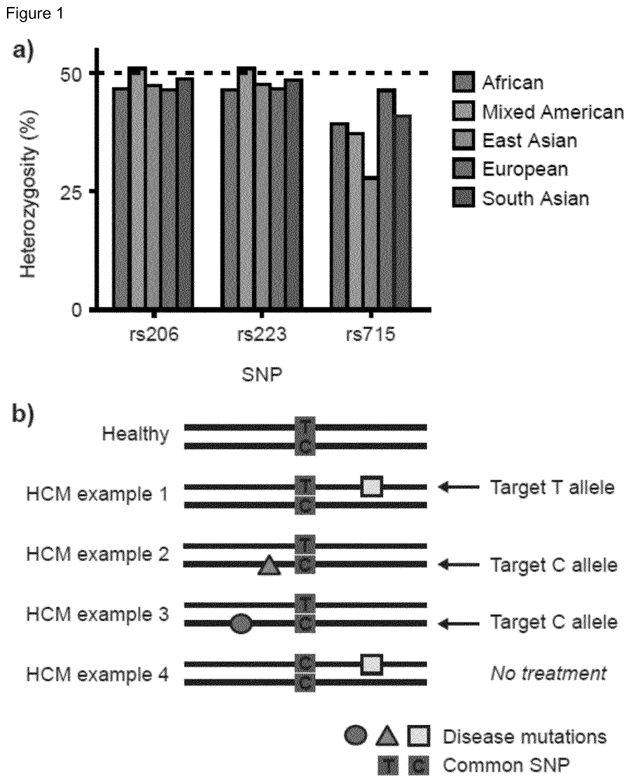

We analyzed the Phase 3 1000 Genomes database [11] to identify SNPs in the human population that occur with high frequency, i.e. genetic coordinates in MYH7 that contain different nucleotides on each allele (i.e. heterozygous base) in a large fraction of people. We found three SNPs with high heterozygosity: rs2239578 (48%), rs2069540 (48%), and rs7157716 (38%) (FIG. 1a). These three common SNPs are found in intron 2, exon 3, and exon 24 of MYH7, respectively, and will be referred to as rs223, rs206, and rs715.

For rs206, the reference nucleotide is cytosine and the SNP is thymine, while for rs223 and rs715 the reference is thymine and the SNP is cytosine (Table 3). The designation of a SNP in these cases is somewhat arbitrary since both the reference and alternate allele are common. No other polymorphisms are found within 25 bases upstream or downstream of each SNP. To each of these SNP regions, locked nucleic acid (LNA) gapmer ASOs were designed and synthesized to selectively knockdown mRNA containing either cytosine or thymine at the SNP coordinate. This strategy depends on the ability of the ASOs to induce robust degradation of the SNP-matched RNA while minimizing degradation of the SNP-mismatched RNA. This allows for multiple disease-linked mutations to be targeted with the same ASO (FIG. 1b). Within each SNP region ASOs were tiled along the transcript, resulting in some ASOs having the position of the SNP in the 5′ end, some in the DNA gap in the middle, and some in the 3′ end. Furthermore, for each position ASOs from 15 to 20 nucleotides in length were designed, with one to four LNA nucleotides in the 5′ end and two to four in the 3′end. Varying the SNP position and ASO structure in this manner resulted in 47 ASOs targeting the rs715 SNP (15-C, 32-T), 111 ASOs targeting the rs223 SNP (52-C, 59-T), and 130 ASOs targeting the rs206 SNP (75-C, 55-T) (Table S1).

### Example 2: In Vitro Knockdown

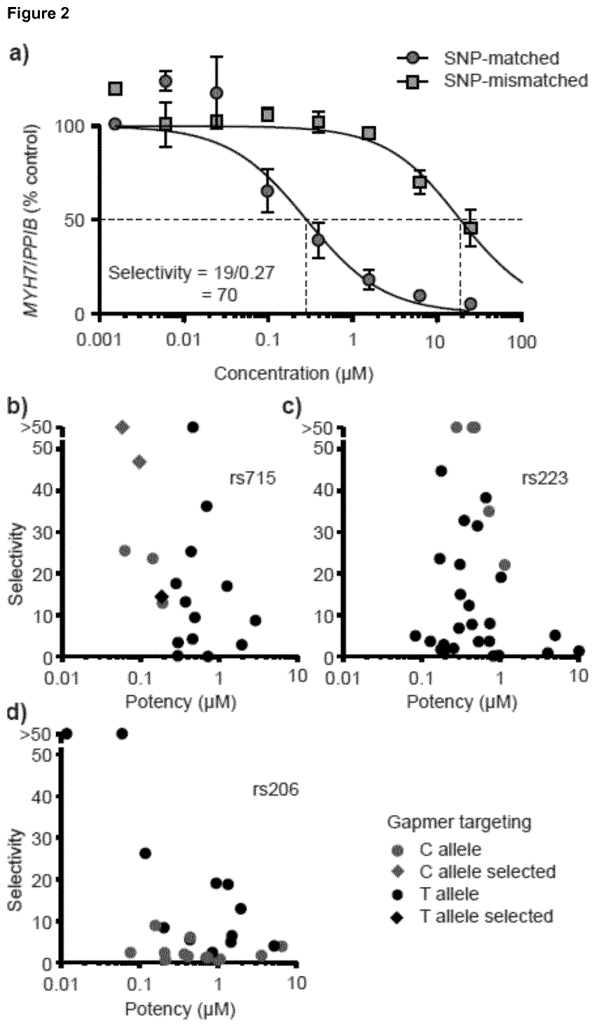

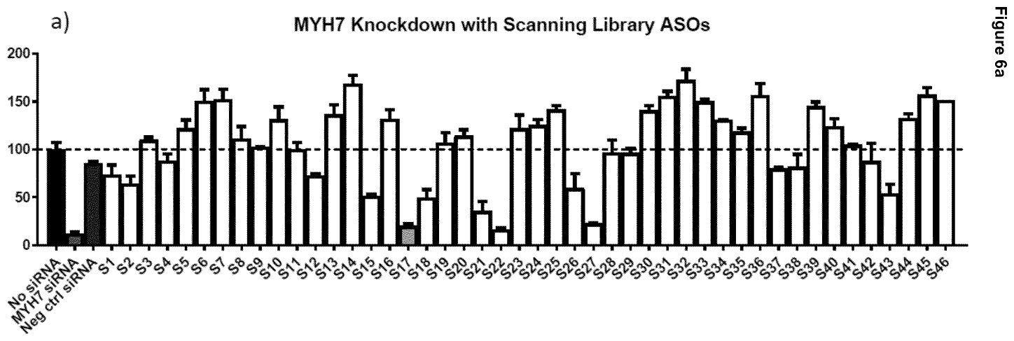

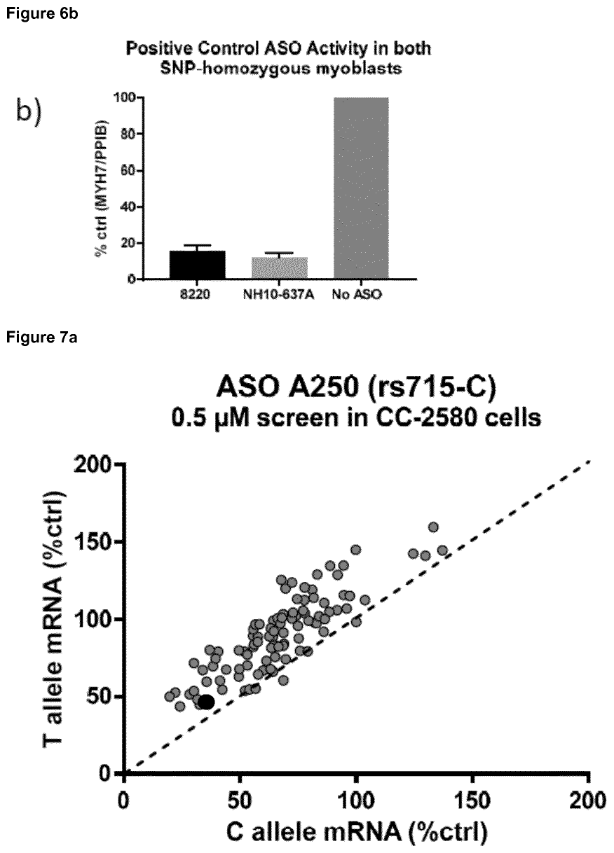

We screened the initial ASO libraries in the QuantiGene 2.0 assay to identify compounds that exhibit good knockdown of MYH7 RNA. Two human skeletal muscle myoblast cell lines were used; both lines were homozygous at each SNP position and the lines were perfectly complementary (e.g. one line had C/C at rs206 and T/T at rs223 and rs715, the other had T/T at rs206 and C/C at rs223 and rs715). ASOs were screened in both cell lines at 5 uM using gymnotic delivery to determine SNP-matched and SNP-mismatched RNA knockdown at a 3 day timepoint. A non-SNP targeting ASO (S17 in FIG. 6) was used as a positive control and showed similar activity in both cell lines (88% and 85% knockdown at 5 uM (FIG. 6). This suggests that ASO uptake is similar between the two human myoblast lines. ASOs that showed mild selectivity (>50% knockdown of MYH7 mRNA in the SNP-matched cell line as well as <25% knockdown in the SNP-mismatched cell line, Table 6) were selected for follow-up potency determination. Additional ASOs that showed good SNP-matched potency but did not meet the selectivity criteria were also progressed to concentration response curves (CRCs). ASO potency values (IC₅₀) were determined from CRCs using the QuantiGene assay in both the SNP-matched and SNP-mismatched cell lines (FIG. 2a). This allows calculation of a selectivity ratio, defined as the ratio of SNP-mismatched potency to SNP-matched potency. FIGS. 2b-2d show that ASOs can be found in all three SNP regions that show good potency and selectivity, highlighting the generalizability of this approach.

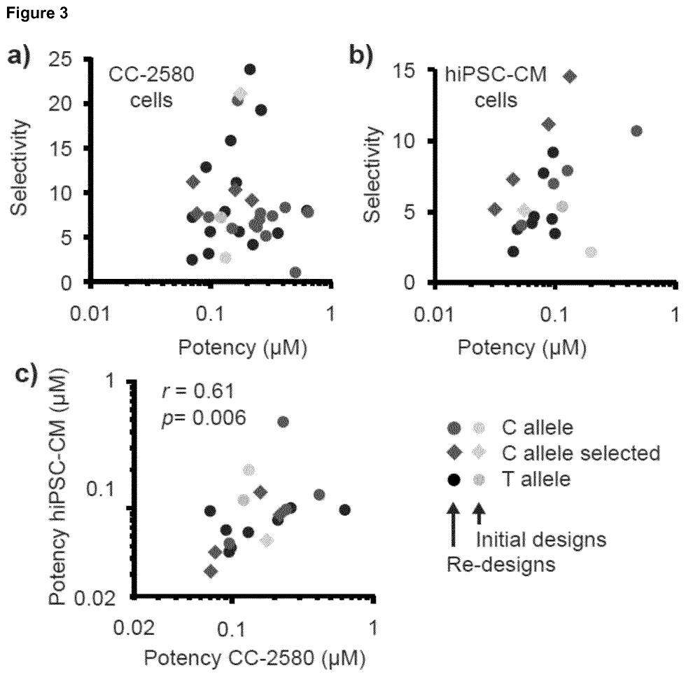

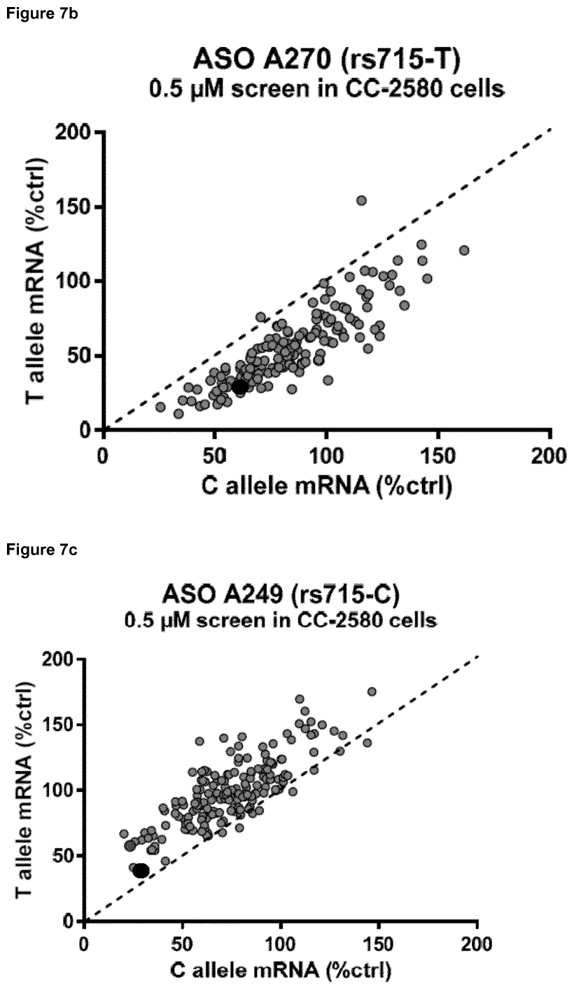

Since allele selectivity was shown at all three SNP regions, we decided to focus on ASOs targeting the rs715 SNP region due to sequence homology between human and dog and cynomolgus monkey. We also developed a droplet digital PCR (ddPCR) assay that enabled us to measure allele-specific mRNA knockdown in cells that are heterozygous at the rs715 SNP position (T on one allele, C on the other). This assay used multiplexed PCR reactions to simultaneously measure allele-specific potency in a SNP-heterozygous human myoblast cell line. We generated 450 LNA gapmer redesigns based on ASOs from the initial rs715 library that exhibited good potency and selectivity (two ASOs, A249 and A250, targeting the rs715-C SNP and one, A270, targeting rs715-T). The redesigns are also shown in Table 5. Transcript start site was maintained, but ASO lengths were varied from 17 to 19 nucleotides. Furthermore, the number and position of LNA modifications within each ASO were varied, with LNA and DNA interspersed. All ASOs had between 4 and 15 consecutive DNAs to allow for RNase H binding and cleavage, with the majority of ASOs containing between 5 and 7 consecutive DNAs. These ASOs were tested at 500 nM in human myoblasts that are heterozygous at the rs715 SNP position (CC-2580 cells, Lonza), with mRNA levels determined 6 days after compound addition (FIGS. 7a-7c and Table 7). From this single point data, a subset of ASOs were selected for follow-up potency determinations in two rs715 SNP-heterozygous cell lines: human myoblasts (CC-2580), data shown in Table 8 and human iPSC-derived cardiomyocytes (iCell², CDI), data shown in Table 9. Potencies and selectivities are summarized in FIG. 3.

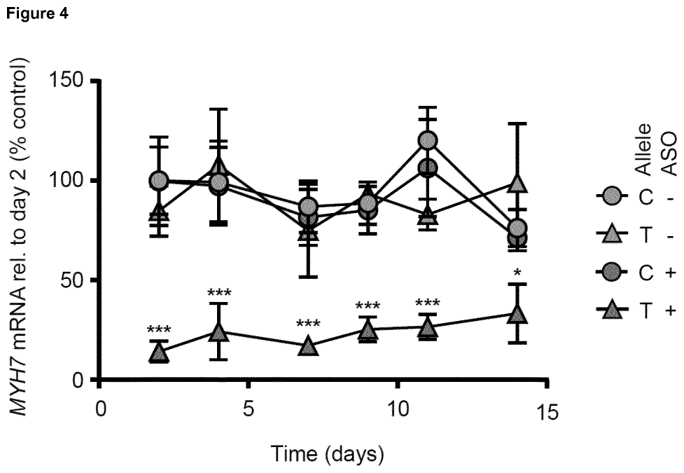

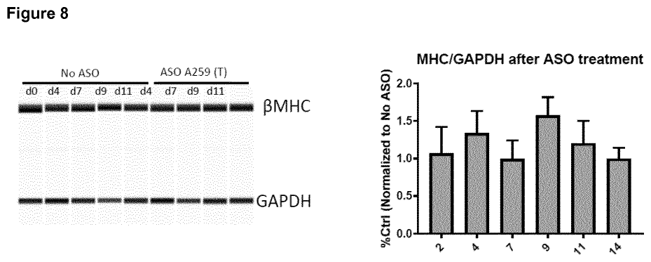

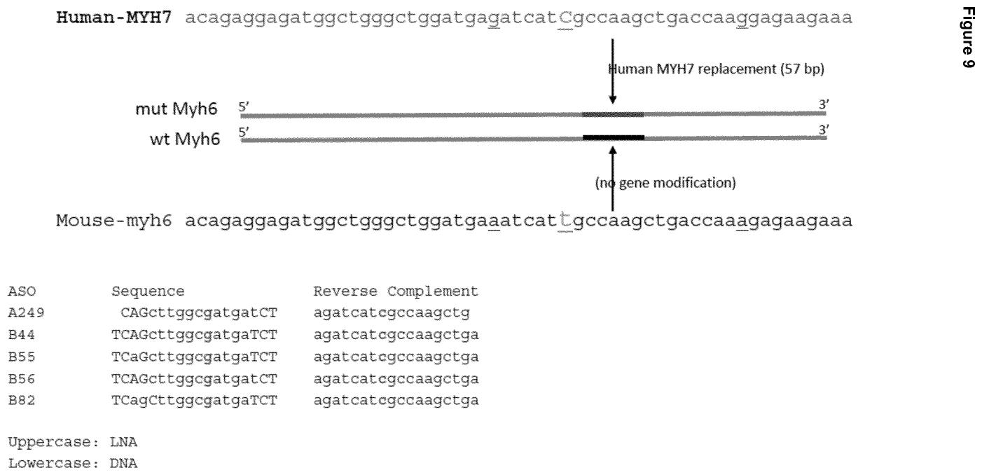

To determine if allele-compensation occurs during allele-selective knockdown, we performed a time course study in iCell² iPSC-CM. These cells are heterozygous at the rs715 SNP position and were treated with 250 nM of ASO A259 (see Table 5 for sequence), a potent and selective ASO targeting the rs715-T SNP. FIG. 4 shows that the ASO does not knockdown the SNP-mismatched allele (rs715-C), but does give strong knockdown of the SNP-matched allele (rs715-T). This experiment shows in vitro allele-selective mRNA knockdown at up to two weeks following ASO addition. These experiments also included replicate cell plates for quantifying the effect of ASO addition on MYH7 protein (β-myosin heavy chain). Protein lysates at all timepoints were probed with an antibody that recognizes β-MHC but not α-MHC (iCell² cells also contain α-MHC, which is encoded by the MYH6 gene). FIG. 8 shows that reduction in β-MHC was not seen at any timepoint, suggesting compensation by the SNP-mismatched allele at the translation level.

### Example 3: In Vivo Knockdown

Further, we were interested in determining if these ASOs could selectively knockdown target mRNA in vivo. We generated a genetically engineered mouse model with the human MYH7 sequence inserted at the rs715 SNP region. Since the predominant myosin isoform in mouse heart is fast α-MHC, the human rs715-C SNP region was inserted into the mouse Myh6 gene. This was a 57 basepair replacement at the rs715 SNP coordinate (i.e. the location of the SNP plus 32 nucleotides upstream and 24 nucleotides downstream; see FIG. 9). This genetic modification did not change the predicted amino acid sequence of mouse α-MHC. This mouse line was heterozygous for the humanized MYH7 fragment, as it contained wildtype Myh6 on the other allele. Five ASOs targeting the rs715-C SNP were tested in vivo in these heterozygous humanized mice. Mouse Myh6 contains a thymine base at the rs715 SNP coordinate, so the rs715-C ASOs are predicted to not target the WT allele. In addition, there is another mismatch six bases upstream of the SNP (guanine in human, adenine in mouse), which gives a two basepair mismatch between the ASO targeting sequences and the wildtype allele (see FIG. 9 for details).

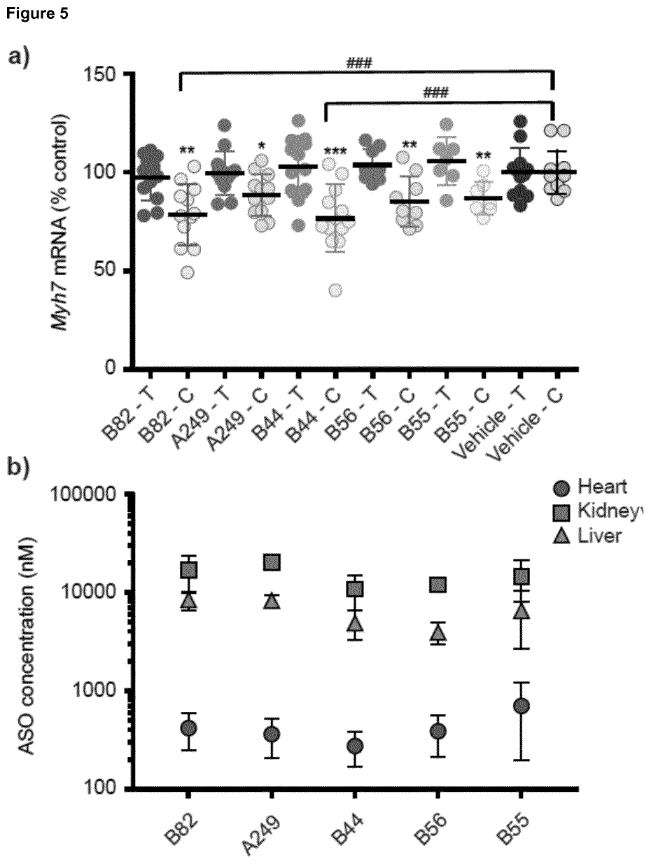

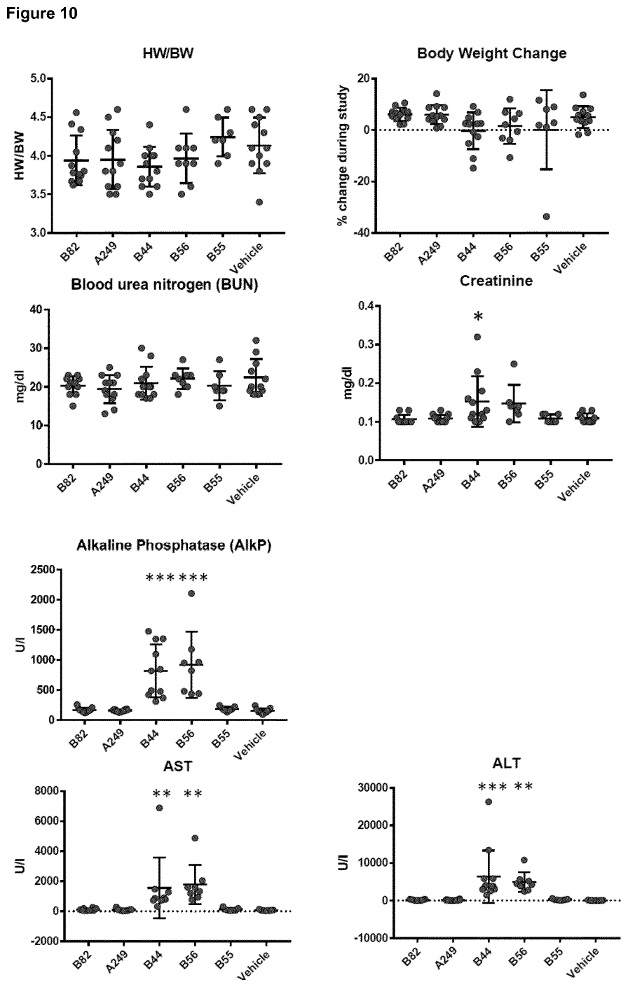

Mice were dosed subcutaneous with 30 mg/kg compound (or saline) on days 0, 1, and 2, and the animals were sacrificed at day 9. Target mRNA knockdown was determined in left ventricular tissue; all five treatment groups had a significant reduction in humanized rs715-C mRNA compared to wildtype Myh6 mRNA (FIG. 5a). In addition, reduction of rs715-C mRNA was significant compared to saline rs715-C in two of the groups (ASOs B82 and B44). This data clearly shows allele-selective knockdown in cardiac tissue. ASO concentrations were determined in left ventricle, kidney, and liver (FIG. 5b) using ASO-specific ELISA assays. As expected, exposure values were significantly higher in kidney and liver. Two of the five compounds were associated with elevation of liver injury markers (AST, ALT, and alkaline phosphatase; FIG. 10).

TABLE 5
Sequences and Compounds

| | | | | | | mRNA | | Pre-mRNA | | |
|---|---|---|---|---|---|---|---|---|---|---|
| SEQ ID NO | SEQUENCE | COMP ID NO # | Compound* | Target seqs | match.to.snp.region | start | end | start | end | Example Figure ID |
| 11 | CGGTCTCGGCAGTGAC | 11 | CGgtctcggcagtGAC | 5, 7 | rs206-c, rs206-c-pre | 306 | 321 | 2161 | 2176 | A1 |
| 12 | TCGGTCTCGGCAGTGAC | 12 | TCGgtctcggcagtGAC | 5, 7 | rs206-c, rs206-c-pre | 306 | 322 | 2161 | 2177 | A2 |
| 13 | TCGGTCTCGGCAGTGA | 13 | TCGgtctcggcagtGA | 5, 7 | rs206-c, rs206-c-pre | 307 | 322 | 2162 | 2177 | A3 |
| 14 | CTCGGTCTCGGCAGTGA | 14 | CTCGgtctcggcagtGA | 5, 7 | rs206-c, rs206-c-pre | 307 | 323 | 2162 | 2178 | A4 |
| 15 | TACTCGGTCTCGGCAGTGA | 15 | TActcggtctcggcagTGA | 5, 7 | rs206-c, rs206-c-pre | 307 | 325 | 2162 | 2180 | A5 |
| 16 | ATACTCGGTCTCGGCAGTGA | 16 | AtactcggtctcggcagtGA | 5, 7 | rs206-c, rs206-c-pre | 307 | 326 | 2162 | 2181 | A6 |
| 17 | ATACTCGGTCTCGGCAGTG | 17 | AtactcggtctcggcagTG | 5, 7 | rs206-c, rs206-c-pre | 308 | 326 | 2163 | 2181 | A7 |
| 18 | CATACTCGGTCTCGGCAGTG | 18 | CatactcggtctcggcagTG | 5, 7 | rs206-c, rs206-c-pre | 308 | 327 | 2163 | 2182 | A8 |
| 19 | ATACTCGGTCTCGGCAGT | 19 | ATActcggtctcggcaGT | 5, 7 | rs206-c, rs206-c-pre | 309 | 326 | 2164 | 2181 | A9 |
| 20 | CATACTCGGTCTCGGCAGT | 20 | CatactcggtctcggcaGT | 5, 7 | rs206-c, rs206-c-pre | 309 | 327 | 2164 | 2182 | A10 |
| 21 | CCATACTCGGTCTCGGCAGT | 21 | CcatactcggtctcggcaGT | 5, 7 | rs206-c, rs206-c-pre | 309 | 328 | 2164 | 2183 | A11 |
| 22 | TACTCGGTCTCGGCAG | 22 | TACtcggtctcggcAG | 5, 7 | rs206-c, rs206-c-pre | 310 | 325 | 2165 | 2180 | A12 |
| 23 | ATACTCGGTCTCGGCAG | 23 | ATActcggtctcggcAG | 5, 7 | rs206-c, rs206-c-pre | 310 | 326 | 2165 | 2181 | A13 |
| 24 | CATACTCGGTCTCGGCAG | 24 | CatactcggtctcggcAG | 5, 7 | rs206-c, rs206-c-pre | 310 | 327 | 2165 | 2182 | A14 |
| 25 | CCATACTCGGTCTCGGCAG | 25 | CcatactcggtctcggcAG | 5, 7 | rs206-c, rs206-c-pre | 310 | 328 | 2165 | 2183 | A15 |
| 26 | ATACTCGGTCTCGGCA | 26 | ATActcggtctcggCA | 5, 7 | rs206-c, rs206-c-pre | 311 | 326 | 2166 | 2181 | A16 |
| 27 | CATACTCGGTCTCGGCA | 27 | CAtactcggtctcggCA | 5, 7 | rs206-c, rs206-c-pre | 311 | 327 | 2166 | 2182 | A17 |
| 28 | CCATACTCGGTCTCGGCA | 28 | CcatactcggtctcggCA | 5, 7 | rs206-c, rs206-c-pre | 311 | 328 | 2166 | 2183 | A18 |
| 29 | ATACTCGGTCTCGGC | 29 | ATActcggtctcgGC | 5, 7 | rs206-c, rs206-c-pre | 312 | 326 | 2167 | 2181 | A19 |
| 30 | CATACTCGGTCTCGGC | 30 | CAtactcggtctcgGC | 5, 7 | rs206-c, rs206-c-pre | 312 | 327 | 2167 | 2182 | A20 |
| 31 | CCATACTCGGTCTCGGC | 31 | CcatactcggtctcgGC | 5, 7 | rs206-c, rs206-c-pre | 312 | 328 | 2167 | 2183 | A21 |
| 32 | GCCATACTCGGTCTCGGC | 32 | GccatactcggtctcgGC | 5, 7 | rs206-c, rs206-c-pre | 312 | 329 | 2167 | 2184 | A22 |
| 33 | TGCCATACTCGGTCTCGGC | 33 | TgccatactcggtctcgGC | 5, 7 | rs206-c, rs206-c-pre | 312 | 330 | 2167 | 2185 | A23 |
| 34 | TTGCCATACTCGGTCTCGGC | 34 | TtgccatactcggtctcgGC | 5, 7 | rs206-c, rs206-c-pre | 312 | 331 | 2167 | 2186 | A24 |
| 35 | CATACTCGGTCTCGG | 35 | CATActcggtctcGG | 5, 7 | rs206-c, rs206-c-pre | 313 | 327 | 2168 | 2182 | A25 |
| 36 | CCATACTCGGTCTCGG | 36 | CCatactcggtctcGG | 5, 7 | rs206-c, rs206-c-pre | 313 | 328 | 2168 | 2183 | A26 |
| 37 | GCCATACTCGGTCTCGG | 37 | GcCatactcggtctcGG | 5, 7 | rs206-c, rs206-c-pre | 313 | 329 | 2168 | 2184 | A27 |
| 38 | TGCCATACTCGGTCTCGG | 38 | TgccatactcggtctcGG | 5, 7 | rs206-c, rs206-c-pre | 313 | 330 | 2168 | 2185 | A28 |
| 39 | TTGCCATACTCGGTCTCGG | 39 | TtgccatactcggtctcGG | 5, 7 | rs206-c, rs206-c-pre | 313 | 331 | 2168 | 2186 | A29 |
| 40 | CTTGCCATACTCGGTCTCGG | 40 | CTtgccatactcggtctcGG | 5, 7 | rs206-c, rs206-c-pre | 313 | 332 | 2168 | 2187 | A30 |
| 41 | CCATACTCGGTCTCG | 41 | CCAtactcggtctCG | 5, 7 | rs206-c, rs206-c-pre | 314 | 328 | 2169 | 2183 | A31 |
| 42 | GCCATACTCGGTCTCG | 42 | GCcatactcggtctCG | 5, 7 | rs206-c, rs206-c-pre | 314 | 329 | 2169 | 2184 | A32 |
| 43 | TGCCATACTCGGTCTCG | 43 | TGccatactcggtctCG | 5, 7 | rs206-c, rs206-c-pre | 314 | 330 | 2169 | 2185 | A33 |
| 44 | TTGCCATACTCGGTCTCG | 44 | TtgccatactcggtctCG | 5, 7 | rs206-c, rs206-c-pre | 314 | 331 | 2169 | 2186 | A34 |
| 45 | CTTGCCATACTCGGTCTCG | 45 | CttgccatactcggtctCG | 5, 7 | rs206-c, rs206-c-pre | 314 | 332 | 2169 | 2187 | A35 |
| 46 | TGCCATACTCGGTCTC | 46 | TGccatactcggtcTC | 5, 7 | rs206-c, rs206-c-pre | 315 | 330 | 2170 | 2185 | A36 |
| 47 | TTGCCATACTCGGTCTC | 47 | TTgccatactcggtcTC | 5, 7 | rs206-c, rs206-c-pre | 315 | 331 | 2170 | 2186 | A37 |
| 48 | CTTGCCATACTCGGTCTC | 48 | CttgccatactcggtcTC | 5, 7 | rs206-c, rs206-c-pre | 315 | 332 | 2170 | 2187 | A38 |
| 49 | TTGCCATACTCGGTCT | 49 | TTGccatactcggtCT | 5, 7 | rs206-c, rs206-c-pre | 316 | 331 | 2171 | 2186 | A39 |
| 50 | CTTGCCATACTCGGTCT | 50 | CttgccatactcggtCT | 5, 7 | rs206-c, rs206-c-pre | 316 | 332 | 2171 | 187 | A40 |
| 51 | CTTGCCATACTCGGTC | 51 | CTTgccatactcggTC | 5, 7 | rs206-c, rs206-c-pre | 317 | 332 | 2172 | 2187 | A41 |
| 52 | TCTTGCCATACTCGGTCTCG | 52 | TCTtgccatactcggtctCG | 5, 7 | rs206-c, rs206-c-pre | 314 | 333 | 2169 | 2188 | A42 |
| 53 | TCTTGCCATACTCGGTCTC | 53 | TCttgccatactcggtcTC | 5, 7 | rs206-c, rs206-c-pre | 315 | 333 | 2170 | 2188 | A43 |
| 54 | GTCTTGCCATACTCGGTCTC | 54 | GTCTtgccatactcggtCTC | 5 | rs206-c | 315 | 334 | | | A44 |
| 55 | TCTTGCCATACTCGGTCT | 55 | TCttgccatactcggtCT | 5, 7 | rs206-c, rs206-c-pre | 316 | 333 | 2171 | 2188 | A45 |
| 56 | GTCTTGCCATACTCGGTCT | 56 | GtcttgccatactcggtCT | 5 | rs206-c | 316 | 334 | | | A46 |
| 57 | TGTCTTGCCATACTCGGTCT | 57 | TGtcttgccatactcggtCT | 5 | rs206-c | 316 | 335 | | | A47 |
| 58 | TCTTGCCATACTCGGTC | 58 | TCttgccatactcggTC | 5, 7 | rs206-c, rs206-c-pre | 317 | 333 | 2172 | 2188 | A48 |
| 59 | GTCTTGCCATACTCGGTC | 59 | GTCTtgccatactcggTC | 5 | rs206-c | 317 | 334 | | | A49 |
| 60 | TCTTGCCATACTCGGT | 60 | TCTtgccatactcgGT | 5, 7 | rs206-c, rs206-c-pre | 318 | 333 | 2173 | 2188 | A50 |
| 61 | GTCTTGCCATACTCGGT | 61 | GTCTtgccatactcgGT | 5 | rs206-c | 318 | 334 | | | A51 |
| 62 | GTCTTGCCATACTCGG | 62 | GTCTtgccatactCGG | 5 | rs206-c | 319 | 334 | | | A52 |
| 63 | TGTCTTGCCATACTCGG | 63 | TGtcttgccatacTCGG | 5 | rs206-c | 319 | 335 | | | A53 |
| 64 | CTGTCTTGCCATACTCGG | 64 | CTgtcttgccatactCGG | 5 | rs206-c | 319 | 336 | | | A54 |
| 65 | CACTGTCTTGCCATACTCGG | 65 | CActgtcttgccatactCGG | 5 | rs206-c | 319 | 338 | | | A55 |
| 66 | CCTTGCCATACTCGGTCTCG | 66 | CcttgccatactcggtctCG | 5, 7 | rs206-c, rs206-c-pre | 314 | 333 | 2169 | 2188 | A56 |
| 67 | CCTTGCCATACTCGGTCTC | 67 | CCttgccatactcggtcTC | 5, 7 | rs206-c, rs206-c-pre | 315 | 333 | 2170 | 2188 | A57 |
| 68 | ACCTTGCCATACTCGGTCTC | 68 | AccttgccatactcggtcTC | 7 | rs206-c-pre | | | 2170 | 2189 | A58 |
| 69 | CCTTGCCATACTCGGTCT | 69 | CcttgccatactcggtCT | 5, 7 | rs206-c, rs206-c-pre | 316 | 333 | 2171 | 2188 | A59 |
| 70 | ACCTTGCCATACTCGGTCT | 70 | AccttgccatactcggtCT | 7 | rs206-c-pre | | | 2171 | 2189 | A60 |
| 71 | CACCTTGCCATACTCGGTCT | 71 | CaccttgccatactcggtCT | 7 | rs206-c-pre | | | 2171 | 2190 | A61 |
| 72 | CCTTGCCATACTCGGTC | 72 | CcttgccatactcggTC | 5, 7 | rs206-c, rs206-c-pre | 317 | 333 | 2172 | 2188 | A62 |
| 73 | ACCTTGCCATACTCGGTC | 73 | AccttgccatactcggTC | 7 | rs206-c-pre | | | 2172 | 2189 | A63 |
| 74 | CACCTTGCCATACTCGGTC | 74 | CaccttgccatactcggTC | 7 | rs206-c-pre | | | 2172 | 2190 | A64 |
| 75 | CCACCTTGCCATACTCGGTC | 75 | CcaccttgccatactcggTC | 7 | rs206-c-pre | | | 2172 | 2191 | A65 |
| 76 | CCTTGCCATACTCGGT | 76 | CCttgccatactcgGT | 5, 7 | rs206-c, rs206-c-pre | 318 | 333 | 2173 | 2188 | A66 |
| 77 | ACCTTGCCATACTCGGT | 77 | ACcttgccatactcgGT | 7 | rs206-c-pre | | | 2173 | 2189 | A67 |
| 78 | CACCTTGCCATACTCGGT | 78 | CaccttgccatactcgGT | 7 | rs206-c-pre | | | 2173 | 2190 | A68 |
| 79 | CCACCTTGCCATACTCGGT | 79 | CcaccttgccatactcgGT | 7 | rs206-c-pre | | | 2173 | 2191 | A69 |
| 80 | CCCACCTTGCCATACTCGGT | 80 | CccaccttgccatactcgGT | 7 | rs206-c-pre | | | 2173 | 2192 | A70 |
| 81 | ACCTTGCCATACTCGG | 81 | ACcttgccatactCGG | 7 | rs206-c-pre | | | 2174 | 2189 | A71 |
| 82 | CACCTTGCCATACTCGG | 82 | CAccttgccatactcGG | 7 | rs206-c-pre | | | 2174 | 2190 | A72 |
| 83 | CCACCTTGCCATACTCGG | 83 | CcaccttgccatactcGG | 7 | rs206-c-pre | | | 2174 | 2191 | A73 |
| 84 | CCCACCTTGCCATACTCGG | 84 | CccaccttgccatactcGG | 7 | rs206-c-pre | | | 2174 | 2192 | A74 |
| 85 | ACCCACCTTGCCATACTCGG | 85 | AcccaccttgccatactcGG | 7 | rs206-c-pre | | | 2174 | 2193 | A75 |
| 86 | TCTTGCCATACTCAGTCT | 86 | TCTtgccatactcagtCT | 6 | rs206-t | 316 | 333 | | | A76 |
| 87 | CCATACTCAGTCTCGGCA | 87 | CcatactcagtctcggCA | 6, 8 | rs206-t, rs206-t-pre | 311 | 328 | 2166 | 2183 | A77 |
| 88 | TTGCCATACTCAGTCTCG | 88 | TTgccatactcagtcTCG | 6, 8 | rs206-t, rs206-t-pre | 314 | 331 | 2169 | 2186 | A78 |
| 89 | TCTTGCCATACTCAGTC | 89 | TCTtgccatactcagTC | 6 | rs206-t | 317 | 333 | | | A79 |
| 90 | CCATACTCAGTCTCGGCAGT | 90 | CcatactcagtctcggcaGT | 6, 8 | rs206-t, rs206-t-pre | 309 | 328 | 2164 | 2183 | A80 |
| 91 | CCATACTCAGTCTCGG | 91 | CCatactcagtctcGG | 6, 8 | rs206-t, rs206-t-pre | 313 | 328 | 2168 | 2183 | A81 |
| 92 | CTTGCCATACTCAGTCTCG | 92 | CTtgccatactcagtctCG | 6, 8 | rs206-t, rs206-t-pre | 314 | 332 | 2169 | 2187 | A82 |
| 93 | CTTGCCATACTCAGTCT | 93 | CTtgccatactcagtCT | 6, 8 | rs206-t, rs206-t-pre | 316 | 332 | 2171 | 2187 | A83 |
| 94 | TTGCCATACTCAGTCTCGGC | 94 | TtgccatactcagtctcgGC | 6, 8 | rs206-t, rs206-t-pre | 312 | 331 | 2167 | 2186 | A84 |
| 95 | TGCCATACTCAGTCTCGG | 95 | TGccatactcagtctcGG | 6, 8 | rs206-t, rs206-t-pre | 313 | 330 | 2168 | 2185 | A85 |
| 96 | CTGTCTTGCCATACTCAG | 96 | CTgtcttgccatactCAG | 6 | rs206-t | 319 | 336 | | | A86 |
| 97 | GCCATACTCAGTCTCGG | 97 | GccatactcagtctcGG | 6, 8 | rs206-t, rs206-t-pre | 313 | 329 | 2168 | 2184 | A87 |
| 98 | CTTGCCATACTCAGTCTC | 98 | CTtgccatactcagtcTC | 6, 8 | rs206-t, rs206-t-pre | 315 | 332 | 2170 | 2187 | A88 |
| 99 | CTTGCCATACTCAGTCTCGG | 99 | CTtgccatactcagtctcGG | 6, 8 | rs206-t, rs206-t-pre | 313 | 332 | 2168 | 2187 | A89 |
| 100 | TTGCCATACTCAGTCTCGG | 100 | TtgccatactcagtctcGG | 6, 8 | rs206-t, rs206-t-pre | 313 | 331 | 2168 | 2186 | A90 |
| 101 | CATACTCAGTCTCGGCAGT | 101 | CatactcagtctcggcaGT | 6, 8 | rs206-t, rs206-t-pre | 309 | 327 | 2164 | 2182 | A91 |
| 102 | CATACTCAGTCTCGGCAGTG | 102 | CatactcagtctcggcagTG | 6, 8 | rs206-t, rs206-t-pre | 308 | 327 | 2163 | 2182 | A92 |
| 103 | TGCCATACTCAGTCTCG | 103 | TGccatactcagtctCG | 6, 8 | rs206-t, rs206-t-pre | 314 | 330 | 2169 | 2185 | A93 |
| 104 | ATACTCAGTCTCGGCAGTG | 104 | AtactcagtctcggcagTG | 6, 8 | rs206-t, rs206-t-pre | 308 | 326 | 2163 | 2181 | A94 |
| 105 | CCATACTCAGTCTCGGCAG | 105 | CcatactcagtctcggcAG | 6, 8 | rs206-t, rs206-t-pre | 310 | 328 | 2165 | 2183 | A95 |
| 106 | CCATACTCAGTCTCGGC | 106 | CcatactcagtctcgGC | 6, 8 | rs206-t, rs206-t-pre | 312 | 328 | 2167 | 2183 | A96 |
| 107 | CACTGTCTTGCCATACTCAG | 107 | CActgtcttgccatactCAG | 6 | rs206-t | 319 | 338 | | | A97 |
| 108 | ATACTCAGTCTCGGCAGT | 108 | ATActcagtctcggcaGT | 6, 8 | rs206-t, rs206-t-pre | 309 | 326 | 2164 | 2181 | A98 |
| 109 | GCCATACTCAGTCTCGGC | 109 | GccatactcagtctcgGC | 6, 8 | rs206-t, rs206-t-pre | 312 | 329 | 2167 | 2184 | A99 |
| 110 | TCTTGCCATACTCAGTCTCG | 110 | TCTtgccatactcagtctCG | 6 | rs206-t | 314 | 333 | | | A100 |
| 111 | CATACTCAGTCTCGGC | 111 | CAtactcagtctcgGC | 6, 8 | rs206-t, rs206-t-pre | 312 | 327 | 2167 | 2182 | A101 |
| 112 | TACTCAGTCTCGGCAG | 112 | TActcagtctcgGcAG | 6, 8 | rs206 t, rs206-t-pre | 310 | 325 | 2165 | 2180 | A102 |
| 113 | GTCTTGCCATACTCAGT | 113 | GtCTtgccatactCAGT | 6 | rs206-t | 318 | 334 | | | A103 |
| 114 | CATACTCAGTCTCGGCA | 114 | CAtactcagtctcggCA | 6, 8 | rs206-t, rs206-t-pre | 311 | 327 | 2166 | 2182 | A104 |
| 115 | ATACTCAGTCTCGGCAG | 115 | ATActcagtctcggcAG | 6, 8 | rs206-t, rs206-t-pre | 310 | 326 | 2165 | 2181 | A105 |
| 116 | TGTCTTGCCATACTCAG | 116 | TGtcttgccatactCAG | 6 | rs206-t | 319 | 335 | | | A106 |
| 117 | TGCCATACTCAGTCTCGGC | 117 | TgccatactcagtctcgGC | 6, 8 | rs206-t, rs206-t-pre | 312 | 330 | 2167 | 2185 | A107 |
| 118 | GCCATACTCAGTCTCG | 118 | GCcatactcagtctCG | 6, 8 | rs206-t, rs206-t-pre | 314 | 329 | 2169 | 2184 | A108 |
| 119 | TTGCCATACTCAGTCTC | 119 | TTgccatactcagtCTC | 6, 8 | rs206-t, rs206-t-pre | 315 | 331 | 2170 | 2186 | A109 |
| 120 | TCTTGCCATACTCAGTCTC | 120 | TCTtgccatactcagtcTC | 6 | rs206-t | 315 | 333 | | | A110 |
| 121 | ACTGTCTTGCCATACTCAG | 121 | ACTgtcttgccatacTCAG | 6 | rs206-t | 319 | 337 | | | A111 |
| 122 | ATACTCAGTCTCGGCA | 122 | ATActcagtctcggCA | 6, 8 | rs206-t, rs206-t-pre | 311 | 326 | 2166 | 2181 | A111 |
| 123 | ATACTCAGTCTCGGCAGTGA | 123 | AtactcagtctcggcagtGA | 6, 8 | rs206-t, rs206-t-pre | 307 | 326 | 2162 | 2181 | A113 |
| 124 | CATACTCAGTCTCGGCAG | 124 | CAtactcagtctcggcAG | 6, 8 | rs206-t, rs206-t-pre | 310 | 327 | 2165 | 2182 | A114 |
| 125 | CCTTGCCATACTCAGTCT | 125 | CCttgccatactcagtCT | 8 | rs206-t-pre | | | 2171 | 2188 | A115 |
| 126 | CACCTTGCCATACTCAGT | 126 | CAccttgccatactCAGT | 8 | rs206-t-pre | | | 2173 | 2190 | A116 |
| 127 | CCTTGCCATACTCAGTC | 127 | CCTtgccatactcagTC | 8 | rs206-t-pre | | | 2172 | 2188 | A117 |
| 128 | CCACCTTGCCATACTCAGT | 128 | CCaccttgccatactCAGT | 8 | rs206-t-pre | | | 2173 | 2191 | A118 |
| 129 | ACCTTGCCATACTCAGT | 129 | ACCTtgccatactcAGT | 8 | rs206-t-pre | | | 2173 | 2189 | A119 |
| 130 | CCACCTTGCCATACTCAG | 130 | CcaccttgccatactcAG | 8 | rs206-t-pre | | | 2174 | 2191 | A120 |
| 131 | CACCTTGCCATACTCAG | 131 | CAccttgccatactcAG | 8 | rs206-t-pre | | | 2174 | 2190 | A121 |
| 132 | ACCCACCTTGCCATACTCAG | 132 | AcccaccttgccatactcAG | 8 | rs206-t-pre | | | 2174 | 2193 | A122 |
| 133 | ACCTTGCCATACTCAGTC | 133 | ACCTtgccatactcagTC | 8 | rs206-t-pre | | | 2172 | 2189 | A123 |
| 134 | CACCTTGCCATACTCAGTCT | 134 | CAccttgccatactcagtCT | 8 | rs206-t-pre | | | 2171 | 2190 | A124 |
| 135 | ACCTTGCCATACTCAGTCT | 135 | AccttgccatactcagtCT | 8 | rs206-t-pre | | | 2171 | 2189 | A125 |
| 136 | ACCTTGCCATACTCAGTCTC | 136 | ACcTtgccatactcagtcTC | 8 | rs206-t-pre | | | 2170 | 2189 | A126 |
| 137 | CCTTGCCATACTCAGTCTCG | 137 | CCTtgccatactcagtctCG | 8 | rs206-t-pre | | | 2169 | 2188 | A127 |
| 138 | CCCACCTTGCCATACTCAGT | 138 | CCcaccttgccatactCAGT | 8 | rs206-t-pre | | | 2173 | 2192 | A128 |
| 139 | CCTTGCCATACTCAGTCTC | 139 | CCTtgccatactcagtcTC | 8 | rs206-t-pre | | | 2170 | 2188 | A129 |
| 140 | CCCACCTTGCCATACTCAG | 140 | CccaccttgccatactcAG | 8 | rs206-t-pre | | | 2174 | 2192 | A130 |
| 141 | ATTTTCAACGCTCTAGC | 141 | ATTTtcaacgctctAGC | 4 | rs223-c | | | 1541 | 1557 | A131 |
| 142 | GATTTTCAACGCTCTAGCT | 142 | GAttttcaacgctctagCT | 4 | rs223-c | | | 1540 | 1558 | A132 |
| 143 | GATTTTCAACGCTCTAGCTT | 143 | GAttttcaacgctctagcTT | 4 | rs223-c | | | 1539 | 1558 | A133 |
| 144 | CTAGATTTTCAACGCTCT | 144 | CTAgattttcaacgctCT | 4 | rs223-c | | | 1544 | 1561 | A134 |
| 145 | GATTTTCAACGCTCTAG | 145 | GATTttcaacgctcTAG | 4 | rs223-c | | | 1542 | 1558 | A135 |
| 146 | TCAACGCTCTAGCTTCAG | 146 | TCAacgctctagcttcAG | 4 | rs223-c | | | 1536 | 1553 | A136 |
| 147 | TTTCAACGCTCTAGCTTCA | 147 | TTtcaacgctctagcttCA | 4 | rs223-c | | | 1537 | 1555 | A137 |
| 148 | CTAGATTTTCAACGCTCTAG | 148 | CTAgattttcaacgctctAG | 4 | rs223-c | | | 1542 | 1561 | A138 |
| 149 | GATTTTCAACGCTCTA | 149 | GATTttcaacgcTCTA | 4 | rs223-c | | | 1543 | 1558 | A139 |
| 150 | TACTAGATTTTCAACGCTC | 150 | TACTagattttcaacgcTC | 4 | rs223-c | | | 1545 | 1563 | A140 |
| 151 | CTAGATTTTCAACGCT | 151 | CTAGattttcaacGCT | 4 | rs223-c | | | 1546 | 1561 | A141 |
| 152 | TTTTCAACGCTCTAGCTT | 152 | TTTtcaacgctctagCTT | 4 | rs223-c | | | 1539 | 1556 | A142 |
| 153 | TTTTCAACGCTCTAGCT | 153 | TTttcaacgctctaGCT | 4 | rs223-c | | | 1540 | 1556 | A143 |
| 154 | TAGATTTTCAACGCTCT | 154 | TAgattttcaacgCTCT | 4 | rs223-c | | | 1544 | 1560 | A144 |
| 155 | AGATTTTCAACGCTCTA | 155 | AGAttttcaacgctCTA | 4 | rs223-c | | | 1543 | 1559 | A145 |
| 156 | TACTAGATTTTCAACGCTCT | 156 | TACtagattttcaacgctCT | 4 | rs223-c | | | 1544 | 1563 | A146 |
| 157 | ACTAGATTTTCAACGCTCTA | 157 | ACtagattttcaacgctCTA | 4 | rs223-c | | | 1543 | 1562 | A147 |
| 158 | ACTAGATTTTCAACGC | 158 | ACTAgattttcaACGC | 4 | rs223-c | | | 1547 | 1562 | A148 |
| 159 | TTTCAACGCTCTAGCTT | 159 | TTTCaacgctctagCTT | 4 | rs223-c | | | 1539 | 1555 | A149 |
| 160 | TTACTAGATTTTCAACGCTC | 160 | TTACtagattttcaacgcTC | 4 | rs223-c | | | 1545 | 1564 | A150 |
| 161 | TTACTAGATTTTCAACGCT | 161 | TTActagattttcaacGCT | 4 | rs223-c | | | 1546 | 1564 | A151 |
| 162 | ATTTTCAACGCTCTAGCTTC | 162 | ATtttcaacgctctagctTC | 4 | rs223-c | | | 1538 | 1557 | A152 |
| 163 | TTTCAACGCTCTAGCTTCAG | 163 | TTtcaacgctctagcttcAG | 4 | rs223-c | | | 1536 | 1555 | A153 |
| 164 | AGATTTTCAACGCTCTAGC | 164 | AGattttcaacgctctaGC | 4 | rs223-c | | | 1541 | 1559 | A154 |
| 165 | ACTAGATTTTCAACGCTC | 165 | ACtagattttcaacGCTC | 4 | rs223-c | | | 1545 | 1562 | A155 |
| 166 | AGATTTTCAACGCTCTAG | 166 | AGattttcaacgctCTAG | 4 | rs223-c | | | 1542 | 1559 | A156 |
| 167 | ACTAGATTTTCAACGCTCT | 167 | ACtagattttcaacgcTCT | 4 | rs223-c | | | 1544 | 1562 | A157 |
| 168 | TTTTCAACGCTCTAGCTTC | 168 | TTttcaacgctctagcTTC | 4 | rs223-c | | | 1538 | 1556 | A158 |
| 169 | TTTCAACGCTCTAGCTTC | 169 | TTTcaacgctctagcTTC | 4 | rs223-c | | | 1538 | 1555 | A159 |
| 170 | TTCAACGCTCTAGCTTCA | 170 | TTcaacgctctagctTCA | 4 | rs223-c | | | 1537 | 1554 | A160 |
| 171 | AGATTTTCAACGCTCTAGCT | 171 | AGattttcaacgctctagCT | 4 | rs223-c | | | 1540 | 1559 | A161 |
| 172 | TACTAGATTTTCAACGC | 172 | TACTagattttcaaCGC | 4 | rs223-c | | | 1547 | 1563 | A162 |
| 173 | TTCAACGCTCTAGCTTCAG | 173 | TTCaacgctctagcttcAG | 4 | rs223-c | | | 1536 | 1554 | A163 |
| 174 | TTTTCAACGCTCTAGCTTCA | 174 | TTTtcaacgctctagcttCA | 4 | rs223-c | | | 1537 | 1556 | A164 |
| 175 | TTTTCAACGCTCTAGC | 175 | TTttcaacgctcTAGC | 4 | rs223-c | | | 1541 | 1556 | A165 |
| 176 | CTTACTAGATTTTCAACGC | 176 | CTtactagattttcaACGC | 4 | rs223-c | | | 1547 | 1565 | A166 |
| 177 | ATTTTCAACGCTCTAGCT | 177 | ATTTtcaacgctctagCT | 4 | rs223-c | | | 1540 | 1557 | A167 |
| 178 | TACTAGATTTTCAACGCT | 178 | TACtagattttcaacGCT | 4 | rs223-c | | | 1546 | 1563 | A168 |
| 179 | TTACTAGATTTTCAACGC | 179 | TTActagattttcaACGC | 4 | rs223-c | | | 1547 | 1564 | A169 |
| 180 | GATTTTCAACGCTCTAGC | 180 | GAttttcaacgctctAGC | 4 | rs223-c | | | 1541 | 1558 | A170 |
| 181 | ATTTTCAACGCTCTAGCTT | 181 | ATTTtcaacgctctagcTT | 4 | rs223-c | | | 1539 | 1557 | A171 |
| 182 | CTAGATTTTCAACGCTCTA | 182 | CTAgattttcaacgctcTA | 4 | rs223-c | | | 1543 | 1561 | A172 |
| 183 | CAACGCTCTAGCTTCAG | 183 | CAacgctctagcttCAG | 4 | rs223-c | | | 1536 | 1552 | A173 |
| 184 | TTCAACGCTCTAGCTTC | 184 | TTcaacgctctagCTTC | 4 | rs223-c | | | 1538 | 1554 | A174 |
| 185 | ACTAGATTTTCAACGCT | 185 | ACtagattttcaaCGCT | 4 | rs223-c | | | 1546 | 1562 | A175 |
| 186 | CTTACTAGATTTTCAACGCT | 186 | CTTactagattttcaacgCT | 4 | rs223-c | | | 1546 | 1565 | A176 |
| 187 | TAGATTTTCAACGCTCTA | 187 | TAgattttcaacgcTCTA | 4 | rs223-c | | | 1543 | 1560 | A177 |
| 188 | TAGATTTTCAACGCTCTAG | 188 | TAgattttcaacgctcTAG | 4 | rs223-c | | | 1542 | 1560 | A178 |
| 189 | TAGATTTTCAACGCTCTAGC | 189 | TAgattttcaacgctctaGC | 4 | rs223-c | | | 1541 | 1560 | A179 |
| 190 | TCTTACTAGATTTTCAACGC | 190 | TCTtactagattttcaacGC | 4 | rs223-c | | | 1547 | 1566 | A180 |
| 191 | CTAGATTTTCAACGCTC | 191 | CTagattttcaacGCTC | 4 | rs223-c | | | 1545 | 1561 | A181 |
| 192 | TTCAACGCTCTAGCTT | 192 | TTCAacgctctagCTT | 4 | rs223-c | | | 1539 | 1554 | A182 |
| 193 | CACTaAGCTTCAGCTTTTC | 193 | CActctagcttcagctttTC | 3 | rs223-t | | | 1530 | 1549 | A183 |
| 194 | CACTCTAGCTTCAGCTTT | 194 | CActctagcttcagCTTT | 3 | rs223-t | | | 1532 | 1549 | A184 |
| 195 | CAACACTCTAGCTTCAGCTT | 195 | CAacactctagcttcagcTT | 3 | rs223-t | | | 1533 | 1552 | A185 |
| 196 | ACACTCTAGCTTCAGCT | 196 | ACActctagcttcagCT | 3 | rs223-t | | | 1534 | 1550 | A186 |
| 197 | AACACTCTAGCTTCAGCT | 197 | AACActctagcttcagCT | 3 | rs223-t | | | 1534 | 1551 | A187 |
| 198 | CAACACTCTAGCTTCAGCT | 198 | CaacactctagcttcagCT | 3 | rs223-t | | | 1534 | 1552 | A188 |
| 199 | TCAACACTCTAGCTTCAGCT | 199 | TCaacactctagcttcagCT | 3 | rs223-t | | | 1534 | 1553 | A189 |
| 200 | AACACTCTAGCTTCAGC | 200 | AACActctagcttcaGC | 3 | rs223-t | | | 1535 | 1551 | A190 |
| 201 | CAACACTCTAGCTTCAGC | 201 | CAacactctagcttcaGC | 3 | rs223-t | | | 1535 | 1552 | A191 |
| 202 | TCAACACTCTAGCTTCAGC | 202 | TCaacactctagcttcaGC | 3 | rs223-t | | | 1535 | 1553 | A192 |
| 203 | TTCAACACTCTAGCTTCAGC | 203 | TtcaacactctagcttcaGC | 3 | rs223-t | | | 1535 | 1554 | A193 |
| 204 | TCAACACTCTAGCTTCAG | 204 | TCAAcactctagcttcAG | 3 | rs223-t | | | 1536 | 1553 | A194 |
| 205 | TTCAACACTCTAGCTTCAG | 205 | TTcaacactctagcttCAG | 3 | rs223-t | | | 1536 | 1554 | A195 |
| 206 | TTTCAACACTCTAGCTTCAG | 206 | TTtcaacactctagcttCAG | 3 | rs223-t | | | 1536 | 1555 | A196 |
| 207 | TCAACACTCTAGCTTCA | 207 | TCaacactctagcTTCA | 3 | rs223-t | | | 1537 | 1553 | A197 |
| 208 | TTCAACACTCTAGCTTCA | 208 | TTcaacactctagcTTCA | 3 | rs223-t | | | 1537 | 1554 | A198 |
| 209 | TTTCAACACTCTAGCTTCA | 209 | TTtcaacactctagcTTCA | 3 | rs223-t | | | 1537 | 1555 | A199 |
| 210 | TTTTCAACACTCTAGCTTCA | 210 | TTttcaacactctagctTCA | 3 | rs223-t | | | 1537 | 1556 | A200 |
| 211 | TTCAACACTCTAGCTTC | 211 | TTCaacactctagCTTC | 3 | rs223-t | | | 1538 | 1554 | A201 |
| 212 | TTTCAACACTCTAGCTTC | 212 | TTTcaacactctagCTTC | 3 | rs223-t | | | 1538 | 1555 | A202 |
| 213 | TTTTCAACACTCTAGCTTC | 213 | TTTTcaacactctagcTTC | 3 | rs223-t | | | 1538 | 1556 | A203 |
| 214 | ATTTTCAACACTCTAGCTTC | 214 | ATTTtcaacactctagctTC | 3 | rs223-t | | | 1538 | 1557 | A204 |
| 215 | TTTCAACACTCTAGCTT | 215 | TTTcaacactctaGCTT | 3 | rs223-t | | | 1539 | 1555 | A205 |
| 216 | TTTTCAACACTCTAGCTT | 216 | TTttcaacactctaGCTT | 3 | rs223-t | | | 1539 | 1556 | A206 |
| 217 | ATTTTCAACACTCTAGCTT | 217 | ATTTtcaacactctagCTT | 3 | rs223-t | | | 1539 | 1557 | A207 |
| 218 | GATTTTCAACACTCTAGCTT | 218 | GAttttcaacactctagCTT | 3 | rs223-t | | | 1539 | 1558 | A208 |
| 219 | TTTTCAACACTCTAGCT | 219 | TTTTcaacactctaGCT | 3 | rs223-t | | | 1540 | 1556 | A209 |
| 220 | ATTTTCAACACTCTAGCT | 220 | ATTttcaacactctaGCT | 3 | rs223-t | | | 1540 | 1557 | A210 |
| 221 | GATTTTCAACACTCTAGCT | 221 | GATtttcaacactctagCT | 3 | rs223-t | | | 1540 | 1558 | A211 |
| 222 | AGATTTTCAACACTCTAGCT | 222 | AGattttcaacactctagCT | 3 | rs223-t | | | 1540 | 1559 | A212 |
| 223 | ATTTTCAACACTCTAGC | 223 | ATTttcaacactcTAGC | 3 | rs223-t | | | 1541 | 1557 | A213 |
| 224 | GATTTTCAACACTCTAGC | 224 | GATTttcaacactctaGC | 3 | rs223-t | | | 1541 | 1558 | A214 |
| 225 | AGATTTTCAACACTCTAGC | 225 | AGattttcaacactctAGC | 3 | rs223-t | | | 1541 | 1559 | A215 |
| 226 | TAGATTTTCAACACTCTAGC | 226 | TAgattttcaacactctaGC | 3 | rs223-t | | | 1541 | 1560 | A216 |
| 227 | GATTTTCAACACTCTAG | 227 | GATTttcaacactCTAG | 3 | rs223-t | | | 1542 | 1558 | A217 |
| 228 | AGATTTTCAACACTCTAG | 228 | AGATtttcaacactcTAG | 3 | rs223-t | | | 1542 | 1559 | A218 |
| 229 | TAGATTTTCAACACTCTAG | 229 | TAgattttcaacactCTAG | 3 | rs223-t | | | 1542 | 1560 | A219 |
| 230 | CTAGATTTTCAACACTCTAG | 230 | CTagattttcaacactcTAG | 3 | rs223-t | | | 1542 | 1561 | A220 |
| 231 | AGATTTTCAACACTCTA | 231 | AGATtttcaacactCTA | 3 | rs223-t | | | 1543 | 1559 | A221 |
| 232 | TAGATTTTCAACACTCTA | 232 | TAGAttttcaacactCTA | 3 | rs223-t | | | 1543 | 1560 | A222 |
| 233 | CTAGATTTTCAACACTCTA | 233 | CTagattttcaacacTCTA | 3 | rs223-t | | | 1543 | 1561 | A223 |
| 234 | ACTAGATTTTCAACACTCTA | 234 | ACtagattttcaacacTCTA | 3 | rs223-t | | | 1543 | 1562 | A224 |
| 235 | TAGATTTTCAACACTCT | 235 | TAGAttttcaacacTCT | 3 | rs223-t | | | 1544 | 1560 | A225 |
| 236 | CTAGATTTTCAACACTCT | 236 | CTAGattttcaacactCT | 3 | rs223-t | | | 1544 | 1561 | A226 |
| 237 | ACTAGATTTTCAACACTCT | 237 | ACtagattttcaacaCTCT | 3 | rs223-t | | | 1544 | 1562 | A227 |
| 238 | TACTAGATTTTCAACACTCT | 238 | TACtagattttcaacacTCT | 3 | rs223-t | | | 1544 | 1563 | A228 |
| 239 | TAGATTTTCAACACTC | 239 | TAGAttttcaacACTC | 3 | rs223-t | | | 1545 | 1560 | A229 |
| 240 | CTAGATTTTCAACACTC | 240 | CTAGattttcaacACTC | 3 | rs223-t | | | 1545 | 1561 | A230 |
| 241 | ACTAGATTTTCAACACTC | 241 | ACTAgattttcaacaCTC | 3 | rs223-t | | | 1545 | 1562 | A231 |
| 242 | TACTAGATTTTCAACACTC | 242 | TACtagattttcaacACTC | 3 | rs223-t | | | 1545 | 1563 | A232 |
| 243 | TTACTAGATTTTCAACACTC | 243 | TTActagattttcaacACTC | 3 | rs223-t | | | 1545 | 1564 | A233 |
| 244 | ACTAGATTTTCAACACT | 244 | ACTAgattttcaaCACT | 3 | rs223-t | | | 1546 | 1562 | A234 |
| 245 | TACTAGATTTTCAACACT | 245 | TACtagattttcaaCACT | 3 | rs223-t | | | 1546 | 1563 | A235 |
| 246 | TTACTAGATTTTCAACACT | 246 | TTActagattttcaaCACT | 3 | rs223-t | | | 1546 | 1564 | A236 |
| 247 | CTTACTAGATTTTCAACACT | 247 | CTTActagattttcaacaCT | 3 | rs223-t | | | 1546 | 1565 | A237 |
| 248 | TACTAGATTTTCAACAC | 248 | TACTagattttcaACAC | 3 | rs223-t | | | 1547 | 1563 | A238 |
| 249 | TTACTAGATTTTCAACAC | 249 | TTACtagattttcaACAC | 3 | rs223-t | | | 1547 | 1564 | A239 |
| 250 | CTTACTAGATTTTCAACAC | 250 | CTTActagattttcaACAC | 3 | rs223-t | | | 1547 | 1565 | A240 |
| 251 | TCTTACTAGATTTTCAACAC | 251 | TCTtactagattttcaACAC | 3 | rs223-t | | | 1547 | 1566 | A241 |
| 252 | CAGCTTGGCGATGATCTC | 252 | CAGCttggcgatgatcTC | 10 | rs715-c | 3090 | 3107 | 12032 | 12049 | A242 |
| 253 | AGCTTGGCGATGATCTCAT | 253 | AGcttggcgatgatctcAT | 10 | rs715-c | 3088 | 3106 | 12030 | 12048 | A243 |
| 254 | TCAGCTTGGCGATGATC | 254 | TCagcttggcgatgATC | 10 | rs715-c | 3092 | 3108 | 12034 | 12050 | A244 |
| 255 | CAGCTTGGCGATGATC | 255 | CAGcttggcgatgATC | 10 | rs715-c | 3092 | 3107 | 12034 | 12049 | A245 |
| 256 | TCAGCTTGGCGATGATCTCA | 256 | TCAGcttggcgatgatCTCA | 10 | rs71S-c | 3089 | 3108 | 12031 | 12050 | A246 |
| 257 | CAGCTTGGCGATGATCTCAT | 257 | CAgcttggcgatgatctcAT | 10 | rs715-c | 3088 | 3107 | 12030 | 12049 | A247 |
| 258 | CTTGGCGATGATCTCAT | 258 | CTTGgcgatgatctcAT | 10 | rs715-c | 3088 | 3104 | 12030 | 12046 | A248 |
| 259 | CAGCTTGGCGATGATCT | 259 | CAGcttggcgatgatCT | 10 | rs715-c | 3091 | 3107 | 12033 | 12049 | A249 |
| 260 | TCAGCTTGGCGATGATCT | 260 | TCagcttggcgatgATCT | 10 | rs715-c | 3091 | 3108 | 12033 | 12050 | A250 |
| 261 | TCAGCTTGGCGATGATCTC | 261 | TCAGcttggcgatgaTCTC | 10 | rs715-c | 3090 | 3108 | 12032 | 12050 | A251 |
| 262 | GCTTGGCGATGATCTCA | 262 | GCttggcgatgatctCA | 10 | rs715-c | 3089 | 3105 | 12031 | 12047 | A252 |
| 263 | AGCTTGGCGATGATCTC | 263 | AGCttggcgatgatcTC | 10 | rs715-c | 3090 | 3106 | 12032 | 12048 | A253 |
| 264 | AGCTTGGCGATGATCTCA | 264 | AGCttggcgatgatctCA | 10 | rs715-c | 3089 | 3106 | 12031 | 12048 | A254 |
| 265 | CAGCTTGGCGATGATCTCA | 265 | CAGCttggcgatgatctCA | 10 | rs715-c | 3089 | 3107 | 12031 | 12049 | A255 |
| 266 | GCTTGGCGATGATCTCAT | 266 | GCttggcgatgatctcAT | 10 | rs715-c | 3088 | 3105 | 12030 | 12047 | A256 |
| 267 | CAATGATCTCATCCAGC | 267 | CAatgatctcatcCAGC | 9 | rs715-t | 3083 | 3099 | 12025 | 12041 | A257 |
| 268 | GCAATGATCTCATCCAGC | 268 | GcaatgatctcatccaGC | 9 | rs715-t | 3083 | 3100 | 12025 | 12042 | A258 |
| 269 | GGCAATGATCTCATCCAGC | 269 | GgCAatgatctcatccaGC | 9 | rs715-t | 3083 | 3101 | 12025 | 12043 | A259 |
| 270 | TGGCAATGATCTCATCCAGC | 270 | TgGcaatgatctcatcCaGC | 9 | rs715-t | 3083 | 3102 | 12025 | 12044 | A260 |
| 271 | GCAATGATCTCATCCAG | 271 | GCaatgatctcatcCAG | 9 | rs715-t | 3084 | 3100 | 12026 | 12042 | A261 |
| 272 | GGCAATGATCTCATCCAG | 272 | GGcaatgatctcatcCAG | 9 | rs715-t | 3084 | 3101 | 12026 | 12043 | A262 |
| 273 | TGGCAATGATCTCATCCAG | 273 | TGGcaatgatctcatcCAG | 9 | rs715-t | 3084 | 3102 | 12026 | 12044 | A263 |
| 274 | GGCAATGATCTCATCCA | 274 | GGcaatgatctcatcCA | 9 | rs715-t | 3085 | 3101 | 12027 | 12043 | A264 |
| 275 | TGGCAATGATCTCATCCA | 275 | TGgcaatgatctcatcCA | 9 | rs715-t | 3085 | 3102 | 12027 | 12044 | A265 |
| 276 | TTGGCAATGATCTCATCCA | 276 | TTGgcaatgatctcatcCA | 9 | rs715-t | 3085 | 3103 | 12027 | 12045 | A266 |
| 277 | CTTGGCAATGATCTCATCCA | 277 | CTtggcaatgatctcaTCCA | 9 | rs715-t | 3085 | 3104 | 12027 | 12046 | A267 |
| 278 | TGGCAATGATCTCATCC | 278 | TGgcaatgatctcaTCC | 9 | rs715-t | 3086 | 3102 | 12028 | 12044 | A268 |
| 279 | TTGGCAATGATCTCATCC | 279 | TTggcaatgatctcATCC | 9 | rs715-t | 3086 | 3103 | 12028 | 12045 | A269 |
| 280 | CTTGGCAATGATCTCATCC | 280 | CTtggcaatgatctcATCC | 9 | rs715-t | 3086 | 3104 | 12028 | 12046 | A270 |
| 281 | GCTTGGCAATGATCTCATCC | 281 | GCttggcaatgatctcatCC | 9 | rs715-t | 3086 | 3105 | 12028 | 12047 | A271 |
| 282 | TTGGCAATGATCTCATC | 282 | TTGGcaatgatctcATC | 9 | rs715-t | 3087 | 3103 | 12029 | 12045 | A272 |
| 283 | CTTGGCAATGATCTCATC | 283 | CTtggcaatgatctCATC | 9 | rs715-t | 3087 | 3104 | 12029 | 12046 | A273 |
| 284 | GCTTGGCAATGATCTCATC | 284 | GCttggcaatgatctcATC | 9 | rs715-t | 3087 | 3105 | 12029 | 12047 | A274 |
| 285 | AGCTTGGCAATGATCTCATC | 285 | AGcttggcaatgatctcATC | 9 | rs715-t | 3087 | 3106 | 12029 | 12048 | A275 |
| 286 | GCTTGGCAATGATCTCAT | 286 | GCTtggcaatgatctcAT | 9 | rs715-t | 3088 | 3105 | 12030 | 12047 | A276 |
| 287 | AGCTTGGCAATGATCTCAT | 287 | AGcttggcaatgatctCAT | 9 | rs715-t | 3088 | 3106 | 12030 | 12048 | A277 |
| 288 | CAGCTTGGCAATGATCTCAT | 288 | CAgcttggcaatgatctcAT | 9 | rs715-t | 3088 | 3107 | 12030 | 12049 | A278 |
| 289 | GCTTGGCAATGATCTCA | 289 | GCttggcaatgatctCA | 9 | rs715-t | 3089 | 3105 | 12031 | 12047 | A279 |
| 290 | AGCTTGGCAATGATCTCA | 290 | AGcttggcaatgatctCA | 9 | rs715-t | 3089 | 3106 | 12031 | 12048 | A280 |
| 291 | CAGCTTGGCAATGATCTCA | 291 | CAgcttggcaatgatctCA | 9 | rs715-t | 3089 | 3107 | 12031 | 12049 | A281 |
| 292 | TCAGCTTGGCAATGATCTCA | 292 | TCAgcttggcaatgatctCA | 9 | rs715-t | 3089 | 3108 | 12031 | 12050 | A282 |
| 293 | AGCTTGGCAATGATCTC | 293 | AGcttggcaatgatCTC | 9 | rs715-t | 3090 | 3106 | 12032 | 12048 | A283 |
| 294 | CAGCTTGGCAATGATCTC | 294 | CAGcttggcaatgatcTC | 9 | rs715-t | 3090 | 3107 | 12032 | 12049 | A284 |
| 295 | TCAGCTTGGCAATGATCTC | 295 | TCAgcttggcaatgatCTC | 9 | rs715-t | 3090 | 3108 | 12032 | 12050 | A285 |
| 296 | CAGCTTGGCAATGATCT | 296 | CAgcttggcaatgaTCT | 9 | rs715-t | 3091 | 3107 | 12033 | 12049 | A286 |
| 297 | TCAGCTTGGCAATGATCT | 297 | TCAgcttggcaatgatCT | 9 | rs715-t | 3091 | 3108 | 12033 | 12050 | A287 |
| 298 | TCAGCTTGGCAATGATC | 298 | TCagcttggcaatGATC | 9 | rs715-t | 3092 | 3108 | 12034 | 12050 | A288 |
| 260 | TCAGCTTGGCGATGATCT | 260,001 | TcaGctTgGcgatgaTCT | 10 | rs715-c | 3091 | 3108 | 12033 | 12050 | B1 |
| 260 | TCAGCTTGGCGATGATCT | 260,002 | TcaGcTtggcgaTgAtCT | 10 | rs715-c | 3091 | 3108 | 12033 | 12050 | B2 |
| 259 | CAGCTTGGCGATGATCT | 259,001 | CAgcttggCgaTgAtCT | 10 | rs715-c | 3091 | 3107 | 12033 | 12049 | B3 |
| 259 | CAGCTTGGCGATGATCT | 259,002 | CAGcTtggcgatgAtCT | 10 | rs715-c | 3091 | 3107 | 12033 | 12049 | B4 |
| 259 | CAGCTTGGCGATGATCT | 259,003 | CAGcttggcgatgATCT | 10 | rs715-c | 3091 | 3107 | 12033 | 12049 | B5 |
| 259 | CAGCTTGGCGATGATCT | 259,004 | CAgcTtggcgatgaTCT | 10 | rs715-c | 3091 | 3107 | 12033 | 12049 | B6 |
| 259 | CAGCTTGGCGATGATCT | 259,005 | CaGcTTgGcgatgatCT | 10 | rs715-c | 3091 | 3107 | 12033 | 12049 | B7 |
| 259 | CAGCTTGGCGATGATCT | 259,006 | CAgcTTggcgatGatCT | 10 | rs715-c | 3091 | 3107 | 12033 | 12049 | B8 |
| 260 | TCAGCTTGGCGATGATCT | 260,003 | TcAgcTtGgCgatgatCT | 10 | rs715-c | 3091 | 3108 | 12033 | 12050 | B9 |
| 259 | CAGCTTGGCGATGATCT | 259,007 | CagCttGgcgatgaTCT | 10 | rs715-c | 3091 | 3107 | 12033 | 12049 | B10 |
| 259 | CAGCTTGGCGATGATCT | 259,008 | CaGcTtggcgaTGatCT | 10 | rs715-c | 3091 | 3107 | 12033 | 12049 | B11 |
| 260 | TCAGCTTGGCGATGATCT | 260,004 | TCagCttggcgatgatCT | 10 | rs715-c | 3091 | 3108 | 12033 | 12050 | B12 |
| 259 | CAGCTTGGCGATGATCT | 259,009 | CAGctTggcgatGatCT | 10 | rs715-c | 3091 | 3107 | 12033 | 12049 | B13 |
| 259 | CAGCTTGGCGATGATCT | 259,010 | CAgCttggcgatgAtCT | 10 | rs715-c | 3091 | 3107 | 12033 | 12049 | B14 |
| 259 | CAGCTTGGCGATGATCT | 259,011 | CagCttggcgAtGaTCT | 10 | rs715-c | 3091 | 3107 | 12033 | 12049 | B15 |
| 259 | CAGCTTGGCGATGATCT | 259,012 | CAGCttggcgatgaTCT | 10 | rs715-c | 3091 | 3107 | 12033 | 12049 | B16 |
| 259 | CAGCTTGGCGATGATCT | 259,013 | CaGcTTgGcgatgaTCT | 10 | rs715-c | 3091 | 3107 | 12033 | 12049 | B17 |
| 259 | CAGCTTGGCGATGATCT | 259,014 | CAgcttggcgaTgAtCT | 10 | rs715-c | 3091 | 3107 | 12033 | 12049 | B18 |
| 259 | CAGCTTGGCGATGATCT | 259,015 | CagCttGgcgatGaTCT | 10 | rs715-c | 3091 | 3107 | 12033 | 12049 | B19 |
| 259 | CAGCTTGGCGATGATCT | 259,016 | CagCTtggCgatgaTCT | 10 | rs715-c | 3091 | 3107 | 12033 | 12049 | B20 |
| 260 | TCAGCTTGGCGATGATCT | 260,005 | TCagcttGgcgatgATCT | 10 | rs715-c | 3091 | 3108 | 12033 | 12050 | B21 |
| 259 | CAGCTTGGCGATGATCT | 259,017 | CaGcTTggcgatgAtCT | 10 | rs715-c | 3091 | 3107 | 12033 | 12049 | B22 |
| 259 | CAGCTTGGCGATGATCT | 259,018 | CaGCttggcgatGatCT | 10 | rs715-c | 3091 | 3107 | 12033 | 12049 | B23 |
| 259 | CAGCTTGGCGATGATCT | 259,019 | CAgcttggcgAtGaTCT | 10 | rs715-c | 3091 | 3107 | 12033 | 12049 | B24 |
| 259 | CAGCTTGGCGATGATCT | 259,020 | CaGCTtggcgAtgatCT | 10 | rs715-c | 3091 | 3107 | 12033 | 12049 | B25 |
| 259 | CAGCTTGGCGATGATCT | 259,021 | CaGCttggcgatgAtCT | 10 | rs71S-c | 3091 | 3107 | 12033 | 12049 | B26 |
| 260 | TCAGCTTGGCGATGATCT | 260,006 | TCagcttggcgatgATCT | 10 | rs715-c | 3091 | 3108 | 12033 | 12050 | B27 |
| 259 | CAGCTTGGCGATGATCT | 259,022 | CagCTtggcgaTGatCT | 10 | rs715-c | 3091 | 3107 | 12033 | 12049 | B28 |
| 260 | TCAGCTTGGCGATGATCT | 260,007 | TcaGcTtGgCgatgatCT | 10 | rs715-c | 3091 | 3108 | 12033 | 12050 | B29 |
| 260 | TCAGCTTGGCGATGATCT | 260,008 | TCagctTggcgatgATCT | 10 | rs715-c | 3091 | 3108 | 12033 | 12050 | B30 |
| 259 | CAGCTTGGCGATGATCT | 259,023 | CagcttggcgATgAtCT | 10 | rs715-c | 3091 | 3107 | 12033 | 12049 | B31 |
| 259 | CAGCTTGGCGATGATCT | 259,024 | CAgCttggcgatgatCT | 10 | rs715-c | 3091 | 3107 | 12033 | 12049 | B32 |
| 259 | CAGCTTGGCGATGATCT | 259,025 | CAgctTggcgaTgAtCT | 10 | rs715-c | 3091 | 3107 | 12033 | 12049 | B33 |
| 259 | CAGCTTGGCGATGATCT | 259,026 | CagCttggcgatGatCT | 10 | rs715-c | 3091 | 3107 | 12033 | 12049 | B34 |
| 260 | TCAGCTTGGCGATGATCT | 260,009 | TcAgCttggcgATgAtCT | 10 | rs715-c | 3091 | 3108 | 12033 | 12050 | B35 |
| 259 | CAGCTTGGCGATGATCT | 259,027 | CAgcttggcgATGatCT | 10 | rs715-c | 3091 | 3107 | 12033 | 12049 | B36 |
| 260 | TCAGCTTGGCGATGATCT | 260,010 | TCagCttggcgatGaTCT | 10 | rs715-c | 3091 | 3108 | 12033 | 12050 | B37 |
| 259 | CAGCTTGGCGATGATCT | 259,028 | CAgcttggcgaTgatCT | 10 | rs715-c | 3091 | 3107 | 12033 | 12049 | B38 |
| 259 | CAGCTTGGCGATGATCT | 259,029 | CAgCttggcgAtgatCT | 10 | rs715-c | 3091 | 3107 | 12033 | 12049 | B39 |
| 259 | CAGCTTGGCGATGATCT | 259,030 | CAgcttggCgatgatCT | 10 | rs715-c | 3091 | 3107 | 12033 | 12049 | B40 |
| 260 | TCAGCTTGGCGATGATCT | 260,011 | TCagcttggCgatgaTCT | 10 | rs715-c | 3091 | 3108 | 12033 | 12050 | B41 |
| 260 | TCAGCTTGGCGATGATCT | 260,012 | TCAGcttggcgatgATCT | 10 | rs715-c | 3091 | 3108 | 12033 | 12050 | B42 |
| 259 | CAGCTTGGCGATGATCT | 259,031 | CAgcttggcgatgaTCT | 10 | rs715-c | 3091 | 3107 | 12033 | 12049 | B43 |
| 260 | TCAGCTTGGCGATGATCT | 260,013 | TCAGcttggcgatgaTCT | 10 | rs715-c | 3091 | 3108 | 12033 | 12050 | B44 |
| 260 | TCAGCTTGGCGATGATCT | 260,014 | TcAgcTTgGcgatgaTCT | 10 | rs715-c | 3091 | 3108 | 12033 | 12050 | B45 |
| 259 | CAGCTTGGCGATGATCT | 259,032 | CaGcTtggcgatGatCT | 10 | rs715-c | 3091 | 3107 | 12033 | 12049 | B46 |
| 259 | CAGCTTGGCGATGATCT | 259,033 | CagCttGgcgatgATCT | 10 | rs715-c | 3091 | 3107 | 12033 | 12049 | B47 |
| 260 | TCAGCTTGGCGATGATCT | 260,015 | TCagcttggcGatgATCT | 10 | rs715-c | 3091 | 3108 | 12033 | 12050 | B48 |
| 259 | CAGCTTGGCGATGATCT | 259,034 | CagCtTggcgatgAtCT | 10 | rs715-c | 3091 | 3107 | 12033 | 12049 | B49 |
| 260 | TCAGCTTGGCGATGATCT | 260,016 | TCAgCttggcgatGaTCT | 10 | rs715-c | 3091 | 3108 | 12033 | 12050 | B50 |
| 260 | TCAGCTTGGCGATGATCT | 260,017 | TCagcttggcGatgaTCT | 10 | rs715-c | 3091 | 3108 | 12033 | 12050 | B51 |
| 260 | TCAGCTTGGCGATGATCT | 260,018 | TcaGcTTggcgatgAtCT | 10 | rs715-c | 3091 | 3108 | 12033 | 12050 | B52 |
| 260 | TCAGCTTGGCGATGATCT | 260,019 | TcaGcTtggcgaTgATCT | 10 | rs715-c | 3091 | 3108 | 12033 | 12050 | B53 |
| 260 | TCAGCTTGGCGATGATCT | 260,020 | TcAgcTtggcgaTgAtCT | 10 | rs715-c | 3091 | 3108 | 12033 | 12050 | B54 |
| 260 | TCAGCTTGGCGATGATCT | 260,021 | TCaGcttggcgatgaTCT | 10 | rs715-c | 3091 | 3108 | 12033 | 12050 | B55 |
| 260 | TCAGCTTGGCGATGATCT | 260,022 | TCAGcttggcgatgatCT | 10 | rs715-c | 3091 | 3108 | 12033 | 12050 | B56 |
| 260 | TCAGCTTGGCGATGATCT | 260,023 | TcAgCttggcgaTgAtCT | 10 | rs715-c | 3091 | 3108 | 12033 | 12050 | B57 |
| 259 | CAGCTTGGCGATGATCT | 259,035 | CAgCtTggcGatgatCT | 10 | rs715-c | 3091 | 3107 | 12033 | 12049 | B58 |
| 259 | CAGCTTGGCGATGATCT | 259,036 | CagCTtggCGatgatCT | 10 | rs715-c | 3091 | 3107 | 12033 | 12049 | B59 |
| 259 | CAGCTTGGCGATGATCT | 259,037 | CagCttGgcgatGAtCT | 10 | rs715-c | 3091 | 3107 | 12033 | 12049 | B60 |
| 259 | CAGCTTGGCGATGATCT | 259,038 | CagCttGgcGatgatCT | 10 | rs715-c | 3091 | 3107 | 12033 | 12049 | B61 |
| 259 | CAGCTTGGCGATGATCT | 259,039 | CAgcttggcgatGatCT | 10 | rs715-c | 3091 | 3107 | 12033 | 12049 | B62 |
| 260 | TCAGCTTGGCGATGATCT | 260,024 | TcaGcttggcgAtgAtCT | 10 | rs715-c | 3091 | 3108 | 12033 | 12050 | B63 |
| 259 | CAGCTTGGCGATGATCT | 259,040 | CagCttggcgaTgAtCT | 10 | rs715-c | 3091 | 3107 | 12033 | 12049 | B64 |
| 259 | CAGCTTGGCGATGATCT | 259,041 | CAgcttggCgatgaTCT | 10 | rs715-c | 3091 | 3107 | 12033 | 12049 | B65 |
| 259 | CAGCTTGGCGATGATCT | 259,042 | CAgCttggcgaTgatCT | 10 | rs715-c | 3091 | 3107 | 12033 | 12049 | B66 |
| 259 | CAGCTTGGCGATGATCT | 259,043 | CagCttGgcgatGatCT | 10 | rs715-c | 3091 | 3107 | 12033 | 12049 | B67 |
| 259 | CAGCTTGGCGATGATCT | 259,044 | CaGcTtggcgatGaTCT | 10 | rs715-c | 3091 | 3107 | 12033 | 12049 | B68 |
| 260 | TCAGCTTGGCGATGATCT | 260,025 | TcaGcTtGgcgatgATCT | 10 | rs715-c | 3091 | 3108 | 12033 | 12050 | B69 |
| 259 | CAGCTTGGCGATGATCT | 259,045 | CagCttGgCGatgatCT | 10 | rs715-c | 3091 | 3107 | 12033 | 12049 | B70 |
| 259 | CAGCTTGGCGATGATCT | 259,046 | CaGCttGgcGatgatCT | 10 | rs715-c | 3091 | 3107 | 12033 | 12049 | B71 |
| 259 | CAGCTTGGCGATGATCT | 259,047 | CaGCttggcgAtgatCT | 10 | rs715-c | 3091 | 3107 | 12033 | 12049 | B72 |
| 260 | TCAGCTTGGCGATGATCT | 260,026 | TcAGcTtGgcgatgaTCT | 10 | rs715-c | 3091 | 3108 | 12033 | 12050 | B73 |
| 259 | CAGCTTGGCGATGATCT | 259,048 | CagCttggCgatgatCT | 10 | rs715-c | 3091 | 3107 | 12033 | 12049 | B74 |
| 260 | TCAGCTTGGCGATGATCT | 260,027 | TcAGctTggcgatgAtCT | 10 | rs715-c | 3091 | 3108 | 12033 | 12050 | B75 |
| 259 | CAGCTTGGCGATGATCT | 259,049 | CAgcttggcgatGaTCT | 10 | rs715-c | 3091 | 3107 | 12033 | 12049 | B76 |
| 259 | CAGCTTGGCGATGATCT | 259,050 | CagCttGgCgatgatCT | 10 | rs715-c | 3091 | 3107 | 12033 | 12049 | B77 |
| 259 | CAGCTTGGCGATGATCT | 259,051 | CagCttggcgAtgatCT | 10 | rs715-c | 3091 | 3107 | 12033 | 12049 | B78 |
| 259 | CAGCTTGGCGATGATCT | 259,052 | CaGcTtggcgatGAtCT | 10 | rs715-c | 3091 | 3107 | 12033 | 12049 | B79 |
| 259 | CAGCTTGGCGATGATCT | 259,052 | CaGcTtggcgatGAtCT | 10 | rs715-c | 3091 | 3107 | 12033 | 12049 | B79 |
| 259 | CAGCTTGGCGATGATCT | 259,053 | CAgcTtgGcgatgaTCT | 10 | rs715-c | 3091 | 3107 | 12033 | 12049 | B80 |
| 259 | CAGCTTGGCGATGATCT | 259,054 | CAgcttGgcgatgAtCT | 10 | rs715-c | 3091 | 3107 | 12033 | 12049 | B81 |
| 260 | TCAGCTTGGCGATGATCT | 260,028 | TCagCttggcgatgaTCT | 10 | rs715-c | 3091 | 3108 | 12033 | 12050 | B82 |
| 260 | TCAGCTTGGCGATGATCT | 260,029 | TcAgCttGgcgatgaTCT | 10 | rs715-c | 3091 | 3108 | 12033 | 12050 | B83 |
| 259 | CAGCTTGGCGATGATCT | 259,055 | CAGcTtggcgatgatCT | 10 | rs715-c | 3091 | 3107 | 12033 | 12049 | B84 |
| 260 | TCAGCTTGGCGATGATCT | 260,030 | TCAgcttggcgatgaTCT | 10 | rs715-c | 3091 | 3108 | 12033 | 12050 | B85 |
| 259 | CAGCTTGGCGATGATCT | 259,056 | CagCtTggCgatgatCT | 10 | rs715-c | 3091 | 3107 | 12033 | 12049 | B86 |
| 260 | TCAGCTTGGCGATGATCT | 260,031 | TCAgcttggcgatgATCT | 10 | rs715-c | 3091 | 3108 | 12033 | 12050 | B87 |
| 260 | TCAGCTTGGCGATGATCT | 260,032 | TcAgcTtGgcgatgaTCT | 10 | rs715-c | 3091 | 3108 | 12033 | 12050 | B88 |
| 259 | CAGCTTGGCGATGATCT | 259,057 | CagCTtggcgAtGatCT | 10 | rs715-c | 3091 | 3107 | 12033 | 12049 | B89 |
| 259 | CAGCTTGGCGATGATCT | 259,058 | CaGCttggcgaTgatCT | 10 | rs715-c | 3091 | 3107 | 12033 | 12049 | B90 |
| 260 | TCAGCTTGGCGATGATCT | 260,033 | TCaGcttggcgatGaTCT | 10 | rs715-c | 3091 | 3108 | 12033 | 12050 | B91 |
| 259 | CAGCTTGGCGATGATCT | 259,059 | CagCTtggcgaTgaTCT | 10 | rs715-c | 3091 | 3107 | 12033 | 12049 | B92 |
| 259 | CAGCTTGGCGATGATCT | 259,060 | CaGctTggcgatgATCT | 10 | rs715-c | 3091 | 3107 | 12033 | 12049 | B93 |
| 259 | CAGCTTGGCGATGATCT | 259,061 | CAgcttggcGatgAtCT | 10 | rs715-c | 3091 | 3107 | 12033 | 12049 | B94 |
| 259 | CAGCTTGGCGATGATCT | 259,062 | CagCTtggcgAtgatCT | 10 | rs715-c | 3091 | 3107 | 12033 | 12049 | B95 |
| 259 | CAGCTTGGCGATGATCT | 259,063 | CAgcTtGgcgatgaTCT | 10 | rs715-c | 3091 | 3107 | 12033 | 12049 | B96 |
| 260 | TCAGCTTGGCGATGATCT | 260,034 | TcAgCTtGgcgatgaTCT | 10 | rs715-c | 3091 | 3108 | 12033 | 12050 | B97 |
| 260 | TCAGCTTGGCGATGATCT | 260,035 | TcAgcTTggcgatGatCT | 10 | rs715-c | 3091 | 3108 | 12033 | 12050 | B98 |
| 260 | TCAGCTTGGCGATGATCT | 260,036 | TCagcttggcgatGaTCT | 10 | rs715-c | 3091 | 3108 | 12033 | 12050 | B99 |
| 259 | CAGCTTGGCGATGATCT | 259,064 | CAgcttggcGatGatCT | 10 | rs715-c | 3091 | 3107 | 12033 | 12049 | B100 |
| 259 | CAGCTTGGCGATGATCT | 259,065 | CagCTtggcgaTgatCT | 10 | rs715-c | 3091 | 3107 | 12033 | 12049 | B101 |
| 259 | CAGCTTGGCGATGATCT | 259,066 | CAgCTtggcgaTgatCT | 10 | rs715-c | 3091 | 3107 | 12033 | 12049 | B102 |
| 259 | CAGCTTGGCGATGATCT | 259,067 | CagCtTggCGatgatCT | 10 | rs715-c | 3091 | 3107 | 12033 | 12049 | B103 |
| 259 | CAGCTTGGCGATGATCT | 259,068 | CAgcTtggcgatgatCT | 10 | rs715-c | 3091 | 3107 | 12033 | 12049 | B104 |
| 259 | CAGCTTGGCGATGATCT | 259,069 | CagCTtgGcgatgaTCT | 10 | rs715-c | 3091 | 3107 | 12033 | 12049 | B105 |
| 259 | CAGCTTGGCGATGATCT | 259,070 | CAgcttggcgaTgaTCT | 10 | rs715-c | 3091 | 3107 | 12033 | 12049 | B106 |
| 259 | CAGCTTGGCGATGATCT | 259,071 | CagCTTggCgatgatCT | 10 | rs715-c | 3091 | 3107 | 12033 | 12049 | B107 |
| 259 | CAGCTTGGCGATGATCT | 259,072 | CaGcttggcgATgaTCT | 10 | rs715-c | 3091 | 3107 | 12033 | 12049 | B108 |
| 260 | TCAGCTTGGCGATGATCT | 260,037 | TcaGctTgGcgatgatCT | 10 | rs715-c | 3091 | 3108 | 12033 | 12050 | B109 |
| 260 | TCAGCTTGGCGATGATCT | 260,038 | TcaGcTTggCgatgatCT | 10 | rs715-c | 3091 | 3108 | 12033 | 12050 | B110 |
| 260 | TCAGCTTGGCGATGATCT | 260,039 | TCaGctTggcgatgATCT | 10 | rs715-c | 3091 | 3108 | 12033 | 12050 | B111 |
| 259 | CAGCTTGGCGATGATCT | 259,073 | CAgctTggcgatgaTCT | 10 | rs715-c | 3091 | 3107 | 12033 | 12049 | B112 |
| 260 | TCAGCTTGGCGATGATCT | 260,040 | TcAgCttggcgAtGatCT | 10 | rs715-c | 3091 | 3108 | 12033 | 12050 | B113 |
| 259 | CAGCTTGGCGATGATCT | 259,074 | CAgcTtgGcgatgatCT | 10 | rs715-c | 3091 | 3107 | 12033 | 12049 | B114 |
| 260 | TCAGCTTGGCGATGATCT | 260,041 | TCagCttggcgAtGatCT | 10 | rs715-c | 3091 | 3108 | 12033 | 12050 | B115 |
| 259 | CAGCTTGGCGATGATCT | 259,075 | CAgcttggcgAtgAtCT | 10 | rs715-c | 3091 | 3107 | 12033 | 12049 | B116 |
| 260 | TCAGCTTGGCGATGATCT | 260,042 | TcAGcTtggcgatGatCT | 10 | rs715-c | 3091 | 3108 | 12033 | 12050 | B117 |
| 259 | CAGCTTGGCGATGATCT | 259,076 | CAgcttggcgatgatCT | 10 | rs715-c | 3091 | 3107 | 12033 | 12049 | B118 |
| 259 | CAGCTTGGCGATGATCT | 259,077 | CagCTTggcgaTgAtCT | 10 | rs715-c | 3091 | 3107 | 12033 | 12049 | B119 |
| 259 | CAGCTTGGCGATGATCT | 259,078 | CAGcttggcgaTgAtCT | 10 | rs715-c | 3091 | 3107 | 12033 | 12049 | B120 |
| 259 | CAGCTTGGCGATGATCT | 259,079 | CaGcttggcgAtGatCT | 10 | rs715-c | 3091 | 3107 | 12033 | 12049 | B121 |
| 259 | CAGCTTGGCGATGATCT | 259,080 | CAgcTtggcgatGatCT | 10 | rs715-c | 3091 | 3107 | 12033 | 12049 | B122 |
| 260 | TCAGCTTGGCGATGATCT | 260,043 | TcAgcTtggcgatGAtCT | 10 | rs715-c | 3091 | 3108 | 12033 | 12050 | B123 |
| 259 | CAGCTTGGCGATGATCT | 259,081 | CAGctTggcgatgatCT | 10 | rs715-c | 3091 | 3107 | 12033 | 12049 | B124 |
| 259 | CAGCTTGGCGATGATCT | 259,082 | CagCTtGgcgatgaTCT | 10 | rs715-c | 3091 | 3107 | 12033 | 12049 | B125 |
| 260 | TCAGCTTGGCGATGATCT | 260,044 | TcaGctTggcgatgATCT | 10 | rs715-c | 3091 | 3108 | 12033 | 12050 | B126 |
| 260 | TCAGCTTGGCGATGATCT | 260,045 | TCagcTtggcgatgaTCT | 10 | rs715-c | 3091 | 3108 | 12033 | 12050 | B127 |
| 259 | CAGCTTGGCGATGATCT | 259,083 | CAgcttGgcgatgatCT | 10 | rs715-c | 3091 | 3107 | 12033 | 12049 | B128 |
| 260 | TCAGCTTGGCGATGATCT | 260,046 | TcaGcTTggcgatGatCT | 10 | rs715-c | 3091 | 3108 | 12033 | 12050 | B129 |
| 259 | CAGCTTGGCGATGATCT | 259,084 | CaGcTtGgCgatgatCT | 10 | rs715-c | 3091 | 3107 | 12033 | 12049 | B130 |
| 259 | CAGCTTGGCGATGATCT | 259,085 | CagCtTggcgatGatCT | 10 | rs715-c | 3091 | 3107 | 12033 | 12049 | B131 |
| 259 | CAGCTTGGCGATGATCT | 259,086 | CAgCttGgcgatGatCT | 10 | rs715-c | 3091 | 3107 | 12033 | 12049 | B132 |
| 259 | CAGCTTGGCGATGATCT | 259,087 | CagCTtGgcgatGatCT | 10 | rs715-c | 3091 | 3107 | 12033 | 12049 | B133 |
| 259 | CAGCTTGGCGATGATCT | 259,088 | CagCttggCGatgatCT | 10 | rs715-c | 3091 | 3107 | 12033 | 12049 | B134 |
| 259 | CAGCTTGGCGATGATCT | 259,089 | CagCTTggcgatGatCT | 10 | rs715-c | 3091 | 3107 | 12033 | 12049 | B135 |
| 259 | CAGCTTGGCGATGATCT | 259,090 | CaGcTtGgcgatgaTCT | 10 | rs715-c | 3091 | 3107 | 12033 | 12049 | B136 |
| 259 | CAGCTTGGCGATGATCT | 259,091 | CAgCTtGgcGatgatCT | 10 | rs715-c | 3091 | 3107 | 12033 | 12049 | B137 |
| 259 | CAGCTTGGCGATGATCT | 259,092 | CagCttGgcgatgAtCT | 10 | rs715-c | 3091 | 3107 | 12033 | 12049 | B138 |
| 260 | TCAGCTTGGCGATGATCT | 260,047 | TcAgcTTggcgatgAtCT | 10 | rs715-c | 3091 | 3108 | 12033 | 12050 | B139 |
| 259 | CAGCTTGGCGATGATCT | 259,093 | CAgcttggCgatgAtCT | 10 | rs715-c | 3091 | 3107 | 12033 | 12049 | B140 |
| 260 | TCAGCTTGGCGATGATCT | 260,048 | TCagcttggcgaTgaTCT | 10 | rs715-c | 3091 | 3108 | 12033 | 12050 | B141 |
| 260 | TCAGCTTGGCGATGATCT | 260,049 | TcaGcTTgGcgatgatCT | 10 | rs715-c | 3091 | 3108 | 12033 | 12050 | B142 |
| 259 | CAGCTTGGCGATGATCT | 259,094 | CaGcTtGgcgatgAtCT | 10 | rs715-c | 3091 | 3107 | 12033 | 12049 | B143 |
| 259 | CAGCTTGGCGATGATCT | 259,095 | CaGctTgGcgatgatCT | 10 | rs715-c | 3091 | 3107 | 12033 | 12049 | B144 |
| 259 | CAGCTTGGCGATGATCT | 259,096 | CagCttggcgATgaTCT | 10 | rs715-c | 3091 | 3107 | 12033 | 12049 | B145 |
| 259 | CAGCTTGGCGATGATCT | 259,097 | CagCttggcgAtgATCT | 10 | rs715-c | 3091 | 3107 | 12033 | 12049 | B146 |
| 259 | CAGCTTGGCGATGATCT | 259,098 | CAGcttggcgatgaTCT | 10 | rs715-c | 3091 | 3107 | 12033 | 12049 | B147 |
| 260 | TCAGCTTGGCGATGATCT | 260,050 | TcaGcTtGgcgatgAtCT | 10 | rs715-c | 3091 | 3108 | 12033 | 12050 | B148 |
| 259 | CAGCTTGGCGATGATCT | 259,099 | CAgcttggcgatgATCT | 10 | rs715-c | 3091 | 3107 | 12033 | 12049 | B149 |
| 260 | TCAGCTTGGCGATGATCT | 260,051 | TCaGcttggcgatgATCT | 10 | rs715-c | 3091 | 3108 | 12033 | 12050 | B150 |
| 259 | CAGCTTGGCGATGATCT | 259,100 | CagCTtgGcgatgatCT | 10 | rs715-c | 3091 | 3107 | 12033 | 12049 | B151 |
| 260 | TCAGCTTGGCGATGATCT | 260,052 | TcAgCttggcgaTgaTCT | 10 | rs715-c | 3091 | 3108 | 12033 | 12050 | B152 |
| 259 | CAGCTTGGCGATGATCT | 259,101 | CAGcttggcgatgAtCT | 10 | rs715-c | 3091 | 3107 | 12033 | 12049 | B153 |
| 259 | CAGCTTGGCGATGATCT | 259,102 | CaGctTggCgatgatCT | 10 | rs715-c | 3091 | 3107 | 12033 | 12049 | B154 |
| 259 | CAGCTTGGCGATGATCT | 259,103 | CagCttggcgatGaTCT | 10 | rs715-c | 3091 | 3107 | 12033 | 12049 | B1S5 |
| 260 | TCAGCTTGGCGATGATCT | 260,053 | TCagcttgGcgatgaTCT | 10 | rs715-c | 3091 | 3108 | 12033 | 12050 | B156 |
| 259 | CAGCTTGGCGATGATCT | 259,104 | CagCTtggcgatGatCT | 10 | rs715-c | 3091 | 3107 | 12033 | 12049 | B157 |
| 260 | TCAGCTTGGCGATGATCT | 260,054 | TCagCttggcgaTgAtCT | 10 | rs715-c | 3091 | 3108 | 12033 | 12050 | B158 |
| 259 | CAGCTTGGCGATGATCT | 259,105 | CAgcttgGcgatgAtCT | 10 | rs715-c | 3091 | 3107 | 12033 | 12049 | B159 |
| 259 | CAGCTTGGCGATGATCT | 259,106 | CagcttggcgATGAtCT | 10 | rs715-c | 3091 | 3107 | 12033 | 12049 | B160 |
| 259 | CAGCTTGGCGATGATCT | 259,107 | CAgcttGgcgatgaTCT | 10 | rs715-c | 3091 | 3107 | 12033 | 12049 | B161 |
| 259 | CAGCTTGGCGATGATCT | 259,108 | CAgCTtggcgAtgatCT | 10 | rs715-c | 3091 | 3107 | 12033 | 12049 | B162 |
| 259 | CAGCTTGGCGATGATCT | 259,109 | CaGCTtggcgatGatCT | 10 | rs715-c | 3091 | 3107 | 12033 | 12049 | B163 |
| 260 | TCAGCTTGGCGATGATCT | 260,055 | TcaGcttggcgAtgATCT | 10 | rs715-c | 3091 | 3108 | 12033 | 12050 | B164 |
| 260 | TCAGCTTGGCGATGATCT | 260,056 | TCagcttggCgatgATCT | 10 | rs715-c | 3091 | 3108 | 12033 | 12050 | B165 |
| 259 | CAGCTTGGCGATGATCT | 259,110 | CagCttggcgATgAtCT | 10 | rs715-c | 3091 | 3107 | 12033 | 12049 | B166 |
| 259 | CAGCTTGGCGATGATCT | 259,111 | CAgCttggcgatGatCT | 10 | rs715-c | 3091 | 3107 | 12033 | 12049 | B167 |
| 259 | CAGCTTGGCGATGATCT | 259,112 | CAgCttggcgatgaTCT | 10 | rs715-c | 3091 | 3107 | 12033 | 12049 | B168 |
| 259 | CAGCTTGGCGATGATCT | 259,113 | CagCTtggcgATgAtCT | 10 | rs715-c | 3091 | 3107 | 12033 | 12049 | B169 |
| 259 | CAGCTTGGCGATGATCT | 259,114 | CagCttggcgatgAtCT | 10 | rs715-c | 3091 | 3107 | 12033 | 12049 | B170 |
| 260 | TCAGCTTGGCGATGATCT | 260,057 | TcAgcTTggCgatgatCT | 10 | rs715-c | 3091 | 3108 | 12033 | 12050 | B171 |
| 259 | CAGCTTGGCGATGATCT | 259,115 | CAGCttggcgatgatCT | 10 | rs715-c | 3091 | 3107 | 12033 | 12049 | B172 |
| 259 | CAGCTTGGCGATGATCT | 259,116 | CAgCttggcgaTgAtCT | 10 | rs715-c | 3091 | 3107 | 12033 | 12049 | B173 |
| 259 | CAGCTTGGCGATGATCT | 259,117 | CagCttggcgaTgATCT | 10 | rs715-c | 3091 | 3107 | 12033 | 12049 | B174 |
| 260 | TCAGCTTGGCGATGATCT | 260,058 | TCagcttggcgatgaTCT | 10 | rs715-c | 3091 | 3108 | 12033 | 12050 | B175 |
| 259 | CAGCTTGGCGATGATCT | 259,118 | CAgcttggcGatGAtCT | 10 | rs715-c | 3091 | 3107 | 12033 | 12049 | B176 |
| 259 | CAGCTTGGCGATGATCT | 259,119 | CAgcTtGgcgatgAtCT | 10 | rs715-c | 3091 | 3107 | 12033 | 12049 | B177 |
| 259 | CAGCTTGGCGATGATCT | 259,120 | CagCTtGgcgatgAtCT | 10 | rs715-c | 3091 | 3107 | 12033 | 12049 | B178 |
| 259 | CAGCTTGGCGATGATCT | 259,121 | CaGctTggcgatgAtCT | 10 | rs715-c | 3091 | 3107 | 12033 | 12049 | B179 |
| 260 | TCAGCTTGGCGATGATCT | 260,059 | TCAgcTtggcgatGaTCT | 10 | rs715-c | 3091 | 3108 | 12033 | 12050 | B180 |
| 260 | TCAGCTTGGCGATGATCT | 260,060 | TCagcttggcgAtgATCT | 10 | rs715-c | 3091 | 3108 | 12033 | 12050 | B181 |
| 260 | TCAGCTTGGCGATGATCT | 260,061 | TcaGcTtggcgatGatCT | 10 | rs715-c | 3091 | 3108 | 12033 | 12050 | B182 |
| 259 | CAGCTTGGCGATGATCT | 259,122 | CagCttggcgATgatCT | 10 | rs715-c | 3091 | 3107 | 12033 | 12049 | B183 |
| 259 | CAGCTTGGCGATGATCT | 259,123 | CagCTTggcgatgAtCT | 10 | rs715-c | 3091 | 3107 | 12033 | 12049 | B184 |
| 260 | TCAGCTTGGCGATGATCT | 260,062 | TcAgcTtGgcgatgAtCT | 10 | rs715-c | 3091 | 3108 | 12033 | 12050 | B185 |
| 259 | CAGCTTGGCGATGATCT | 259,124 | CagCttggcgATGatCT | 10 | rs715-c | 3091 | 3107 | 12033 | 12049 | B186 |
| 260 | TCAGCTTGGCGATGATCT | 260,063 | TcaGcTtgGcgatgaTCT | 10 | rs715-c | 3091 | 3108 | 12033 | 12050 | B187 |
| 259 | CAGCTTGGCGATGATCT | 259,125 | CaGcTtggcgaTgAtCT | 10 | rs715-c | 3091 | 3107 | 12033 | 12049 | B188 |
| 259 | CAGCTTGGCGATGATCT | 259,126 | CAgcttggcgATgAtCT | 10 | rs715-c | 3091 | 3107 | 12033 | 12049 | B189 |
| 260 | TCAGCTTGGCGATGATCT | 260,064 | TCagcTtggcgatgATCT | 10 | rs715-c | 3091 | 3108 | 12033 | 12050 | B190 |
| 259 | CAGCTTGGCGATGATCT | 259,127 | CagCtTggcgaTgAtCT | 10 | rs715-c | 3091 | 3107 | 12033 | 12049 | B191 |
| 259 | CAGCTTGGCGATGATCT | 259,128 | CagCTTggcgaTgatCT | 10 | rs715-c | 3091 | 3107 | 12033 | 12049 | B192 |
| 260 | TCAGCTTGGCGATGATCT | 260,065 | TCagCttggcgaTgatCT | 10 | rs715-c | 3091 | 3108 | 12033 | 12050 | B193 |
| 259 | CAGCTTGGCGATGATCT | 259,129 | CAgcttggcgAtgaTCT | 10 | rs715-c | 3091 | 3107 | 12033 | 12049 | B194 |
| 260 | TCAGCTTGGCGATGATCT | 260,066 | TcaGcTtGgcgatgaTCT | 10 | rs715-c | 3091 | 3108 | 12033 | 12050 | B195 |
| 260 | TCAGCTTGGCGATGATCT | 260,067 | TcAgcTTgGcgatgatCT | 10 | rs715-c | 3091 | 3108 | 12033 | 12050 | B196 |
| 259 | CAGCTTGGCGATGATCT | 259,130 | CAgcTtggcgaTgAtCT | 10 | rs715-c | 3091 | 3107 | 12033 | 12049 | B197 |
| 260 | TCAGCTTGGCGATGATCT | 260,068 | TcAgCttGgcgatgAtCT | 10 | rs715-c | 3091 | 3108 | 12033 | 12050 | B198 |
| 259 | CAGCTTGGCGATGATCT | 259,131 | CagCTtggcgatGAtCT | 10 | rs715-c | 3091 | 3107 | 12033 | 12049 | B199 |
| 260 | TCAGCTTGGCGATGATCT | 260,069 | cAgcTtggcgaTgaTCT | 10 | rs715-c | 3091 | 3108 | 12033 | 12050 | B200 |
| 259 | CAGCTTGGCGATGATCT | 259,132 | CagCttggcgAtgAtCT | 10 | rs715-c | 3091 | 3107 | 12033 | 12049 | B201 |
| 259 | CAGCTTGGCGATGATCT | 259,133 | CAgCTtggcgatGatCT | 10 | rs715-c | 3091 | 3107 | 12033 | 12049 | B202 |
| 259 | CAGCTTGGCGATGATCT | 259,134 | CAgcttgGcgatgatCT | 10 | rs715-c | 3091 | 3107 | 12033 | 12049 | B203 |
| 259 | CAGCTTGGCGATGATCT | 259,135 | CagCttggcgatgatCT | 10 | rs715-c | 3091 | 3107 | 12033 | 12049 | B204 |
| 260 | TCAGCTTGGCGATGATCT | 260,070 | TcaGcttggcgATgAtCT | 10 | rs715-c | 3091 | 3108 | 12033 | 12050 | B205 |
| 259 | CAGCTTGGCGATGATCT | 259,136 | CaGcTtggcgaTgaTCT | 10 | rs715-c | 3091 | 3107 | 12033 | 12049 | B206 |
| 259 | CAGCTTGGCGATGATCT | 259,137 | CaGCttggCgatgatCT | 10 | rs715-c | 3091 | 3107 | 12033 | 12049 | B207 |
| 259 | CAGCTTGGCGATGATCT | 259,138 | CagCtTggcGatgatCT | 10 | rs715-c | 3091 | 3107 | 12033 | 12049 | B208 |
| 260 | TCAGCTTGGCGATGATCT | 260,071 | TCagCttggcgatGatCT | 10 | rs715-c | 3091 | 3108 | 12033 | 12050 | B209 |
| 260 | TCAGCTTGGCGATGATCT | 260,072 | TcaGctTggcgatGatCT | 10 | rs715-c | 3091 | 3108 | 12033 | 12050 | B210 |
| 260 | TCAGCTTGGCGATGATCT | 260,073 | TCagCttGgcgatgATCT | 10 | rs715-c | 3091 | 3108 | 12033 | 12050 | B211 |
| 260 | TCAGCTTGGCGATGATCT | 260,074 | TcaGcTTgGcgatgaTCT | 10 | rs715-c | 3091 | 3108 | 12033 | 12050 | B212 |
| 259 | CAGCTTGGCGATGATCT | 259,139 | CagCttggcgaTgaTCT | 10 | rs715-c | 3091 | 3107 | 12033 | 12049 | B213 |
| 259 | CAGCTTGGCGATGATCT | 259,140 | CAgcttggcgAtGatCT | 10 | rs715-c | 3091 | 3107 | 12033 | 12049 | B214 |
| 259 | CAGCTTGGCGATGATCT | 259,141 | CAgcttggcGAtgAtCT | 10 | rs715-c | 3091 | 3107 | 12033 | 12049 | B215 |
| 259 | CAGCTTGGCGATGATCT | 259,142 | CAgCTtggCgatgatCT | 10 | rs715-c | 3091 | 3107 | 12033 | 12049 | B216 |
| 259 | CAGCTTGGCGATGATCT | 259,143 | CaGcTTggCgatgatCT | 10 | rs715-c | 3091 | 3107 | 12033 | 12049 | B217 |
| 259 | CAGCTTGGCGATGATCT | 259,144 | CagCttggcgAtGAtCT | 10 | rs715-c | 3091 | 3107 | 12033 | 12049 | B218 |
| 259 | CAGCTTGGCGATGATCT | 259,145 | CagCTtggcgatGaTCT | 10 | rs715-c | 3091 | 3107 | 12033 | 12049 | B219 |
| 260 | TCAGCTTGGCGATGATCT | 260,075 | TcaGcTtggcgatGAtCT | 10 | rs715-c | 3091 | 3108 | 12033 | 12050 | B220 |
| 260 | TCAGCTTGGCGATGATCT | 260,076 | TcAGctTggcgatGatCT | 10 | rs715-c | 3091 | 3108 | 12033 | 12050 | B221 |
| 259 | CAGCTTGGCGATGATCT | 259,146 | CaGcTTggcgatGatCT | 10 | rs715-c | 3091 | 3107 | 12033 | 12049 | B222 |
| 259 | CAGCTTGGCGATGATCT | 259,147 | CAgcTtggcgatgAtCT | 10 | rs715-c | 3091 | 3107 | 12033 | 12049 | B223 |
| 259 | CAGCTTGGCGATGATCT | 259,148 | CaGcttggcgAtgAtCT | 10 | rs715-c | 3091 | 3107 | 12033 | 12049 | B224 |
| 259 | CAGCTTGGCGATGATCT | 259,149 | CAgctTggcgatgAtCT | 10 | rs715-c | 3091 | 3107 | 12033 | 12049 | B225 |
| 259 | CAGCTTGGCGATGATCT | 259,150 | CaGcttggcgATgAtCT | 10 | rs715-c | 3091 | 3107 | 12033 | 12049 | B226 |
| 260 | TCAGCTTGGCGATGATCT | 260,077 | TcaGcTtggcgaTgaTCT | 10 | rs715-c | 3091 | 3108 | 12033 | 12050 | B227 |
| 259 | CAGCTTGGCGATGATCT | 259,151 | CAgcttggcGaTgAtCT | 10 | rs715-c | 3091 | 3107 | 12033 | 12049 | B228 |
| 260 | TCAGCTTGGCGATGATCT | 260,078 | TcaGcTtgGcgatgatCT | 10 | rs715-c | 3091 | 3108 | 12033 | 12050 | B229 |
| 259 | CAGCTTGGCGATGATCT | 259,152 | CagCttggcgaTgatCT | 10 | rs715-c | 3091 | 3107 | 12033 | 12049 | B230 |
| 259 | CAGCTTGGCGATGATCT | 259,153 | CAgCttggcgATgatCT | 10 | rs715-c | 3091 | 3107 | 12033 | 12049 | B231 |
| 259 | CAGCTTGGCGATGATCT | 259,154 | CaGcTtgGcgatgatCT | 10 | rs715-c | 3091 | 3107 | 12033 | 12049 | B232 |
| 260 | TCAGCTTGGCGATGATCT | 260,079 | TCagcttGgcgatgaTCT | 10 | rs715-c | 3091 | 3108 | 12033 | 12050 | B233 |
| 259 | CAGCTTGGCGATGATCT | 259,155 | CagCTtggCgatgatCT | 10 | rs715-c | 3091 | 3107 | 12033 | 12049 | B234 |
| 260 | TCAGCTTGGCGATGATCT | 260,080 | TcaGctTggCgatgatCT | 10 | rs715-c | 3091 | 3108 | 12033 | 12050 | B235 |
| 260 | TCAGCTTGGCGATGATCT | 260,081 | TCAgCttGgcgatGaTCT | 10 | rs715-c | 3091 | 3108 | 12033 | 12050 | B236 |
| 259 | CAGCTTGGCGATGATCT | 259,156 | CAgcttggcGAtGatCT | 10 | rs715-c | 3091 | 3107 | 12033 | 12049 | B237 |
| 259 | CAGCTTGGCGATGATCT | 259,157 | CAgCttggcgAtGatCT | 10 | rs715-c | 3091 | 3107 | 12033 | 12049 | B238 |
| 260 | TCAGCTTGGCGATGATCT | 260,082 | TCagcttggcgaTgATCT | 10 | rs715-c | 3091 | 3108 | 12033 | 12050 | B239 |
| 259 | CAGCTTGGCGATGATCT | 259,158 | CAgcttgGcgaTgAtCT | 10 | rs715-c | 3091 | 3107 | 12033 | 12049 | B240 |
| 259 | CAGCTTGGCGATGATCT | 259,159 | CagCttgGCgatgatCT | 10 | rs715-c | 3091 | 3107 | 12033 | 12049 | B241 |
| 259 | CAGCTTGGCGATGATCT | 259,160 | CAgcttggcgAtgATCT | 10 | rs715-c | 3091 | 3107 | 12033 | 12049 | B242 |
| 259 | CAGCTTGGCGATGATCT | 259,161 | CAgcttggCgatGatCT | 10 | rs715-c | 3091 | 3107 | 12033 | 12049 | B243 |
| 260 | TCAGCTTGGCGATGATCT | 260,083 | TCAgcTtggcgatGatCT | 10 | rs715-c | 3091 | 3108 | 12033 | 12050 | B244 |
| 259 | CAGCTTGGCGATGATCT | 259,162 | CagCTtGgCgatgatCT | 10 | rs715-c | 3091 | 3107 | 12033 | 12049 | B245 |
| 259 | CAGCTTGGCGATGATCT | 259,163 | CaGcTtgGcgatgaTCT | 10 | rs715-c | 3091 | 3107 | 12033 | 12049 | B246 |
| 260 | TCAGCTTGGCGATGATCT | 260,084 | TcAgCttggcgAtgAtCT | 10 | rs715-c | 3091 | 3108 | 12033 | 12050 | B247 |
| 260 | TCAGCTTGGCGATGATCT | 260,085 | TcAgcTtgGcgatgatCT | 10 | rs715-c | 3091 | 3108 | 12033 | 12050 | B248 |
| 259 | CAGCTTGGCGATGATCT | 259,164 | CaGctTgGcgatgaTCT | 10 | rs715-c | 3091 | 3107 | 12033 | 12049 | B249 |
| 259 | CAGCTTGGCGATGATCT | 259,165 | CagCttggcgAtGatCT | 10 | rs715-c | 3091 | 3107 | 12033 | 12049 | B250 |
| 259 | CAGCTTGGCGATGATCT | 259,166 | CagCttggcgaTGatCT | 10 | rs715-c | 3091 | 3107 | 12033 | 12049 | B251 |
| 259 | CAGCTTGGCGATGATCT | 259,167 | CAgcttggcgatgAtCT | 10 | rs715-c | 3091 | 3107 | 12033 | 12049 | B252 |
| 259 | CAGCTTGGCGATGATCT | 259,168 | CAgctTggcgatgatCT | 10 | rs715-c | 3091 | 3107 | 12033 | 12049 | B253 |
| 260 | TCAGCTTGGCGATGATCT | 260,086 | TCagctTggcgatgaTCT | 10 | rs715-c | 3091 | 3108 | 12033 | 12050 | B254 |
| 259 | CAGCTTGGCGATGATCT | 259,169 | CAgcttGgcgaTgAtCT | 10 | rs715-c | 3091 | 3107 | 12033 | 12049 | B255 |
| 259 | CAGCTTGGCGATGATCT | 259,170 | CagCTtggcgaTgAtCT | 10 | rs715-c | 3091 | 3107 | 12033 | 12049 | B256 |
| 259 | CAGCTTGGCGATGATCT | 259,171 | CagCttggcgatGAtCT | 10 | rs715-c | 3091 | 3107 | 12033 | 12049 | B257 |
| 260 | TCAGCTTGGCGATGATCT | 260,087 | TcAGcttggcgAtGatCT | 10 | rs715-c | 3091 | 3108 | 12033 | 12050 | B258 |
| 260 | TCAGCTTGGCGATGATCT | 260,088 | TcAgcTtggcgatGatCT | 10 | rs715-c | 3091 | 3108 | 12033 | 12050 | B259 |
| 260 | TCAGCTTGGCGATGATCT | 260,089 | TCagcttggcgAtgaTCT | 10 | rs715-c | 3091 | 3108 | 12033 | 12050 | B260 |
| 259 | CAGCTTGGCGATGATCT | 259,172 | CaGctTggcgatGatCT | 10 | rs715-c | 3091 | 3107 | 12033 | 12049 | B261 |
| 260 | TCAGCTTGGCGATGATCT | 260,090 | TcAgcTtGgcgatgATCT | 10 | rs715-c | 3091 | 3108 | 12033 | 12050 | B262 |
| 260 | TCAGCTTGGCGATGATCT | 260,091 | TcaGctTggcgatGAtCT | 10 | rs715-c | 3091 | 3108 | 12033 | 12050 | B263 |
| 259 | CAGCTTGGCGATGATCT | 259,173 | CAGcTtggcgatGatCT | 10 | rs715-c | 3091 | 3107 | 12033 | 12049 | B264 |
| 260 | TCAGCTTGGCGATGATCT | 260,092 | TCagCttggcgAtgAtCT | 10 | rs715-c | 3091 | 3108 | 12033 | 12050 | B265 |
| 259 | CAGCTTGGCGATGATCT | 259,174 | CAgcttgGcgatgaTCT | 10 | rs715-c | 3091 | 3107 | 12033 | 12049 | B266 |
| 259 | CAGCTTGGCGATGATCT | 259,175 | CAGcttggcgatGatCT | 10 | rs715-c | 3091 | 3107 | 12033 | 12049 | B267 |
| 260 | TCAGCTTGGCGATGATCT | 260,093 | TCagCTtggcgatGatCT | 10 | rs715-c | 3091 | 3108 | 12033 | 12050 | B268 |
| 259 | CAGCTTGGCGATGATCT | 259,176 | CagCTtGgcGatgatCT | 10 | rs715-c | 3091 | 3107 | 12033 | 12049 | B269 |
| 259 | CAGCTTGGCGATGATCT | 259,177 | CagCTtggcgATgatCT | 10 | rs715-c | 3091 | 3107 | 12033 | 12049 | B270 |
| 259 | CAGCTTGGCGATGATCT | 259,178 | CAGcTtggcgatgaTCT | 10 | rs715-c | 3091 | 3107 | 12033 | 12049 | B271 |
| 260 | TCAGCTTGGCGATGATCT | 260,094 | TCagcttggcgatgatCT | 10 | rs715-c | 3091 | 3108 | 12033 | 12050 | B272 |
| 260 | TCAGCTTGGCGATGATCT | 260,095 | TcaGcttggcgAtGatCT | 10 | rs715-c | 3091 | 3108 | 12033 | 12050 | B273 |
| 260 | TCAGCTTGGCGATGATCT | 260,096 | TCagcttgGcgatgATCT | 10 | rs715-c | 3091 | 3108 | 12033 | 12050 | B274 |
| 259 | CAGCTTGGCGATGATCT | 259,179 | CAgCttGgcGatgatCT | 10 | rs715-c | 3091 | 3107 | 12033 | 12049 | B275 |
| 260 | TCAGCTTGGCGATGATCT | 260,097 | TcAgcTtgGcgatgaTCT | 10 | rs715-c | 3091 | 3108 | 12033 | 12050 | B276 |
| 259 | CAGCTTGGCGATGATCT | 259,180 | CaGctTGgcgatgaTCT | 10 | rs715-c | 3091 | 3107 | 12033 | 12049 | B277 |
| 259 | CAGCTTGGCGATGATCT | 259,181 | CAgcttggcGatgaTCT | 10 | rs715-c | 3091 | 3107 | 12033 | 12049 | B278 |
| 260 | TCAGCTTGGCGATGATCT | 260,098 | TCAgcttggcgaTgATCT | 10 | rs715-c | 3091 | 3108 | 12033 | 12050 | B279 |
| 260 | TCAGCTTGGCGATGATCT | 260,099 | TCaGcTtggcgatgATCT | 10 | rs715-c | 3091 | 3108 | 12033 | 12050 | B280 |
| 259 | CAGCTTGGCGATGATCT | 259,182 | CAgcttggcGatgatCT | 10 | rs715-c | 3091 | 3107 | 12033 | 12049 | B281 |
| 259 | CAGCTTGGCGATGATCT | 259,183 | CaGCttggcgatgatCT | 10 | rs715-c | 3091 | 3107 | 12033 | 12049 | B282 |
| 259 | CAGCTTGGCGATGATCT | 259,184 | CagCtTggcgaTgatCT | 10 | rs715-c | 3091 | 3107 | 12033 | 12049 | B283 |
| 259 | CAGCTTGGCGATGATCT | 259,185 | CaGcttggcgAtgATCT | 10 | rs715-c | 3091 | 3107 | 12033 | 12049 | B284 |
| 259 | CAGCTTGGCGATGATCT | 259,186 | CAGCttggcgatgATCT | 10 | rs715-c | 3091 | 3107 | 12033 | 12049 | B285 |
| 259 | CAGCTTGGCGATGATCT | 259,187 | CAgCttggCgatgatCT | 10 | rs715-c | 3091 | 3107 | 12033 | 12049 | B286 |
| 259 | CAGCTTGGCGATGATCT | 259,188 | CAgcttggcgAtgatCT | 10 | rs715-c | 3091 | 3107 | 12033 | 12049 | B287 |
| 259 | CAGCTTGGCGATGATCT | 259,189 | CagCTtggcGatgatCT | 10 | rs715-c | 3091 | 3107 | 12033 | 12049 | B288 |
| 260 | TCAGCTTGGCGATGATCT | 260,100 | TcaGctTggcgatgAtCT | 10 | rs715-c | 3091 | 3108 | 12033 | 12050 | B289 |
| 260 | TCAGCTTGGCGATGATCT | 260,101 | TCAGctTggcgatGaTCT | 10 | rs715-c | 3091 | 3108 | 12033 | 12050 | B290 |
| 280 | CTTGGCAATGATCTCATCC | 280,001 | CTtgGcaatgatCtcATCC | 9 | rs715-t | 3086 | 3104 | 12028 | 12046 | B291 |
| 280 | CTTGGCAATGATCTCATCC | 280,002 | CtTggCaatgatCtcAtCC | 9 | rs715-t | 3086 | 3104 | 12028 | 12046 | B292 |
| 280 | CTTGGCAATGATCTCATCC | 280,003 | CttggcaaTgAtcTcAtCC | 9 | rs715-t | 3086 | 3104 | 12028 | 12046 | B293 |
| 280 | CTTGGCAATGATCTCATCC | 280,004 | CTTGgcaatgatctcatCC | 9 | rs715-t | 3086 | 3104 | 12028 | 12046 | B294 |
| 280 | CTTGGCAATGATCTCATCC | 280,005 | CttggCaatgatctCatCC | 9 | rs715-t | 3086 | 3104 | 12028 | 12046 | B295 |
| 280 | CTTGGCAATGATCTCATCC | 280,006 | CTtggcaatgAtctCaTCC | 9 | rs715-t | 3086 | 3104 | 12028 | 12046 | B296 |
| 280 | CTTGGCAATGATCTCATCC | 280,007 | CTtggcaaTgATctCatCC | 9 | rs715-t | 3086 | 3104 | 12028 | 12046 | B297 |
| 280 | CTTGGCAATGATCTCATCC | 280,008 | CTTggcaatgatcTcaTCC | 9 | rs715-t | 3086 | 3104 | 12028 | 12046 | B298 |
| 280 | CTTGGCAATGATCTCATCC | 280,009 | CttggCaatgatctCAtCC | 9 | rs715-t | 3086 | 3104 | 12028 | 12046 | B299 |
| 280 | CTTGGCAATGATCTCATCC | 280,010 | CttGgCaatgatCtcatCC | 9 | rs715-t | 3086 | 3104 | 12028 | 12046 | B300 |
| 280 | CTTGGCAATGATCTCATCC | 280,011 | CTtggcAatgatCtcAtCC | 9 | rs715-t | 3086 | 3104 | 12028 | 12046 | B301 |
| 280 | CTTGGCAATGATCTCATCC | 280,012 | CttGgcaatgAtCtcatCC | 9 | rs715-t | 3086 | 3104 | 12028 | 12046 | B302 |
| 280 | CTTGGCAATGATCTCATCC | 280,013 | CTtggcaatGatctCaTCC | 9 | rs715-t | 3086 | 3104 | 12028 | 12046 | B303 |
| 280 | CTTGGCAATGATCTCATCC | 280,014 | CTtggcaatGaTctCatCC | 9 | rs715-t | 3086 | 3104 | 12028 | 12046 | B304 |
| 280 | CTTGGCAATGATCTCATCC | 280,015 | CttgGcaatgatCtcatCC | 9 | rs715-t | 3086 | 3104 | 12028 | 12046 | B305 |
| 280 | CTTGGCAATGATCTCATCC | 280,016 | CttGgCaatgatctCatCC | 9 | rs715-t | 3086 | 3104 | 12028 | 12046 | B306 |
| 280 | CTTGGCAATGATCTCATCC | 280,017 | CttggcaAtgatCtcAtCC | 9 | rs715-t | 3086 | 3104 | 12028 | 12046 | B307 |
| 280 | CTTGGCAATGATCTCATCC | 280,018 | CTTggcaatgATctCatCC | 9 | rs715-t | 3086 | 3104 | 12028 | 12046 | B308 |
| 280 | CTTGGCAATGATCTCATCC | 280,019 | CtTggCaatgatcTcAtCC | 9 | rs715-t | 3086 | 3104 | 12028 | 12046 | B309 |
| 280 | CTTGGCAATGATCTCATCC | 280,020 | CttggcaATgaTctCatCC | 9 | rs715-t | 3086 | 3104 | 12028 | 12046 | B310 |
| 280 | CTTGGCAATGATCTCATCC | 280,021 | CttggcaatgAtCtcAtCC | 9 | rs715-t | 3086 | 3104 | 12028 | 12046 | B311 |
| 280 | CTTGGCAATGATCTCATCC | 280,022 | CttGgcaatgATctCatCC | 9 | rs715-t | 3086 | 3104 | 12028 | 12046 | B312 |
| 280 | CTTGGCAATGATCTCATCC | 280,023 | CTtggcaatGatcTcaTCC | 9 | rs715-t | 3086 | 3104 | 12028 | 12046 | B313 |
| 280 | CTTGGCAATGATCTCATCC | 280,024 | CTtggcaAtgatCtcatCC | 9 | rs715-t | 3086 | 3104 | 12028 | 12046 | B314 |
| 280 | CTTGGCAATGATCTCATCC | 280,025 | CttggcaatgatCtcAtCC | 9 | rs715-t | 3086 | 3104 | 12028 | 12046 | B315 |
| 280 | CTTGGCAATGATCTCATCC | 280,026 | CTtggcaatgatctcatCC | 9 | rs715-t | 3086 | 3104 | 12028 | 12046 | B316 |
| 280 | CTTGGCAATGATCTCATCC | 280,027 | CttggcAatgatCtcAtCC | 9 | rs715-t | 3086 | 3104 | 12028 | 12046 | B317 |
| 280 | CTTGGCAATGATCTCATCC | 280,028 | CTtggcaatgatCtCaTCC | 9 | rs715-t | 3086 | 3104 | 12028 | 12046 | B318 |
| 280 | CTTGGCAATGATCTCATCC | 280,029 | CttggcaatgatCtcATCC | 9 | rs715-t | 3086 | 3104 | 12028 | 12046 | B319 |
| 280 | CTTGGCAATGATCTCATCC | 280,030 | CtTggcaatgatCtcAtCC | 9 | rs715-t | 3086 | 3104 | 12028 | 12046 | B320 |
| 280 | CTTGGCAATGATCTCATCC | 280,031 | CttGgcAatgatCtcatCC | 9 | rs715-t | 3086 | 3104 | 12028 | 12046 | B321 |
| 280 | CTTGGCAATGATCTCATCC | 280,032 | CTTGgcaatgatctcATCC | 9 | rs715-t | 3086 | 3104 | 12028 | 12046 | B322 |
| 280 | CTTGGCAATGATCTCATCC | 280,033 | CttggcAatgatCtCatCC | 9 | rs715-t | 3086 | 3104 | 12028 | 12046 | B323 |
| 280 | CTTGGCAATGATCTCATCC | 280,034 | CTtggcaatgatctCaTCC | 9 | rs715-t | 3086 | 3104 | 12028 | 12046 | B324 |
| 280 | CTTGGCAATGATCTCATCC | 280,035 | CtTggcaatgATcTcAtCC | 9 | rs715-t | 3086 | 3104 | 12028 | 12046 | B325 |
| 280 | CTTGGCAATGATCTCATCC | 280,036 | CttGgcaatgaTcTcAtCC | 9 | rs715-t | 3086 | 3104 | 12028 | 12046 | B326 |
| 280 | CTTGGCAATGATCTCATCC | 280,037 | CTTggCaatgatCtcAtCC | 9 | rs715-t | 3086 | 3104 | 12028 | 12046 | B327 |
| 280 | CTTGGCAATGATCTCATCC | 280,038 | CttggcaATgAtcTcAtCC | 9 | rs715-t | 3086 | 3104 | 12028 | 12046 | B328 |
| 280 | CTTGGCAATGATCTCATCC | 280,039 | CttggcaaTgATcTcAtCC | 9 | rs715-t | 3086 | 3104 | 12028 | 12046 | B329 |
| 280 | CTTGGCAATGATCTCATCC | 280,040 | CttggcaaTgaTctCatCC | 9 | rs715-t | 3086 | 3104 | 12028 | 12046 | B330 |
| 280 | CTTGGCAATGATCTCATCC | 280,041 | CttggcaaTgATctCatCC | 9 | rs715-t | 3086 | 3104 | 12028 | 12046 | B331 |
| 280 | CTTGGCAATGATCTCATCC | 280,042 | CTtggcAatgatctcATCC | 9 | rs715-t | 3086 | 3104 | 12028 | 12046 | B332 |
| 280 | CTTGGCAATGATCTCATCC | 280,043 | CtTggcAatgatCtcAtCC | 9 | rs715-t | 3086 | 3104 | 12028 | 12046 | B333 |
| 280 | CTTGGCAATGATCTCATCC | 280,044 | CTtggcaatgatcTcATCC | 9 | rs715-t | 3086 | 3104 | 12028 | 12046 | B334 |
| 280 | CTTGGCAATGATCTCATCC | 280,045 | CTtggcAatgatCtCatCC | 9 | rs715-t | 3086 | 3104 | 12028 | 12046 | B335 |
| 280 | CTTGGCAATGATCTCATCC | 280,046 | CTtggcaatGaTcTcAtCC | 9 | rs715-t | 3086 | 3104 | 12028 | 12046 | B336 |
| 280 | CTTGGCAATGATCTCATCC | 280,047 | CttggcaAtgAtCtcatCC | 9 | rs715-t | 3086 | 3104 | 12028 | 12046 | B337 |
| 280 | CTTGGCAATGATCTCATCC | 280,048 | CTtggcaatgAtctcATCC | 9 | rs715-t | 3086 | 3104 | 12028 | 12046 | B338 |
| 280 | CTTGGCAATGATCTCATCC | 280,049 | CTtggcaAtgatctcATCC | 9 | rs715-t | 3086 | 3104 | 12028 | 12046 | B339 |
| 280 | CTTGGCAATGATCTCATCC | 280,050 | CttggcaatGatctCAtCC | 9 | rs715-t | 3086 | 3104 | 12028 | 12046 | B340 |
| 280 | CTTGGCAATGATCTCATCC | 280,051 | CttggcAatgAtCtcatCC | 9 | rs715-t | 3086 | 3104 | 12028 | 12046 | B341 |
| 280 | CTTGGCAATGATCTCATCC | 280,052 | CTtGgcaatgatctcATCC | 9 | rs715-t | 3086 | 3104 | 12028 | 12046 | B342 |
| 280 | CTTGGCAATGATCTCATCC | 280,053 | CttggcaatgaTCtcatCC | 9 | rs715-t | 3086 | 3104 | 12028 | 12046 | B343 |
| 280 | CTTGGCAATGATCTCATCC | 280,054 | CttggcAAtGatCtcatCC | 9 | rs715-t | 3086 | 3104 | 12028 | 12046 | B344 |
| 280 | CTTGGCAATGATCTCATCC | 280,055 | CTtggcaAtgatcTcaTCC | 9 | rs715-t | 3086 | 3104 | 12028 | 12046 | B345 |
| 280 | CTTGGCAATGATCTCATCC | 280,056 | CTtgGcaatgatCtCatCC | 9 | rs715-t | 3086 | 3104 | 12028 | 12046 | B346 |
| 280 | CTTGGCAATGATCTCATCC | 280,057 | CTtggcaatgatcTCaTCC | 9 | rs715-t | 3086 | 3104 | 12028 | 12046 | B347 |
| 280 | CTTGGCAATGATCTCATCC | 280,058 | CttggcaatGatCtcAtCC | 9 | rs715-t | 3086 | 3104 | 12028 | 12046 | B348 |
| 280 | CTTGGCAATGATCTCATCC | 280,059 | CtTgGcaatgaTctCatCC | 9 | rs715-t | 3086 | 3104 | 12028 | 12046 | B349 |
| 280 | CTTGGCAATGATCTCATCC | 280,060 | CTtggcaatGAtcTcAtCC | 9 | rs715-t | 3086 | 3104 | 12028 | 12046 | B350 |
| 280 | CTTGGCAATGATCTCATCC | 280,061 | CttggcAatgatCtcaTCC | 9 | rs715-t | 3086 | 3104 | 12028 | 12046 | B351 |
| 280 | CTTGGCAATGATCTCATCC | 280,062 | CttGgcaatgaTcTCatCC | 9 | rs715-t | 3086 | 3104 | 12028 | 12046 | B352 |
| 280 | CTTGGCAATGATCTCATCC | 280,063 | CttggcAAtgAtCtcatCC | 9 | rs715-t | 3086 | 3104 | 12028 | 12046 | B353 |
| 280 | CTTGGCAATGATCTCATCC | 280,064 | CttggcaatgatctCAtCC | 9 | rs715-t | 3086 | 3104 | 12028 | 12046 | B354 |
| 280 | CTTGGCAATGATCTCATCC | 280,065 | CTtggcaaTgatctcATCC | 9 | rs715-t | 3086 | 3104 | 12028 | 12046 | B355 |
| 280 | CTTGGCAATGATCTCATCC | 280,066 | CTTggCaatgatcTcAtCC | 9 | rs715-t | 3086 | 3104 | 12028 | 12046 | B356 |
| 280 | CTTGGCAATGATCTCATCC | 280,067 | CtTgGcaatgatCtcAtCC | 9 | rs715-t | 3086 | 3104 | 12028 | 12046 | B357 |
| 280 | CTTGGCAATGATCTCATCC | 280,068 | CttggcaaTgatCtcAtCC | 9 | rs715-t | 3086 | 3104 | 12028 | 12046 | B358 |
| 280 | CTTGGCAATGATCTCATCC | 280,069 | CTTGgcaatgatctcaTCC | 9 | rs715-t | 3086 | 3104 | 12028 | 12046 | B359 |
| 280 | CTTGGCAATGATCTCATCC | 280,070 | CttggcAatgatCtcATCC | 9 | rs715-t | 3086 | 3104 | 12028 | 12046 | B360 |
| 280 | CTTGGCAATGATCTCATCC | 280,071 | CttggcaAtgatcTCatCC | 9 | rs715-t | 3086 | 3104 | 12028 | 12046 | B361 |
| 280 | CTTGGCAATGATCTCATCC | 280,072 | CTtgGcaatgatCTcAtCC | 9 | rs715-t | 3086 | 3104 | 12028 | 12046 | B362 |
| 280 | CTTGGCAATGATCTCATCC | 280,073 | CttGgCaatgatcTCatCC | 9 | rs715-t | 3086 | 3104 | 12028 | 12046 | B363 |
| 280 | CTTGGCAATGATCTCATCC | 280,074 | CTtggcaaTgAtcTcAtCC | 9 | rs715-t | 3086 | 3104 | 12028 | 12046 | B364 |
| 280 | CTTGGCAATGATCTCATCC | 280,075 | CTTggcaatGatCtcAtCC | 9 | rs715-t | 3086 | 3104 | 12028 | 12046 | B365 |
| 280 | CTTGGCAATGATCTCATCC | 280,076 | CTtGgCaatgatcTcAtCC | 9 | rs715-t | 3086 | 3104 | 12028 | 12046 | B366 |
| 280 | CTTGGCAATGATCTCATCC | 280,077 | CttGgcaatgATcTcAtCC | 9 | rs715-t | 3086 | 3104 | 12028 | 12046 | B367 |
| 280 | CTTGGCAATGATCTCATCC | 280,078 | CttGgcaatgAtCtcAtCC | 9 | rs715-t | 3086 | 3104 | 12028 | 12046 | B368 |
| 280 | CTTGGCAATGATCTCATCC | 280,079 | CttggcaAtgAtcTcAtCC | 9 | rs715-t | 3086 | 3104 | 12028 | 12046 | B369 |
| 280 | CTTGGCAATGATCTCATCC | 280,080 | CttGgcaatgatCtcAtCC | 9 | rs715-t | 3086 | 3104 | 12028 | 12046 | B370 |
| 280 | CTTGGCAATGATCTCATCC | 280,081 | CttggcAatgatCtcatCC | 9 | rs715-t | 3086 | 3104 | 12028 | 12046 | B371 |
| 280 | CTTGGCAATGATCTCATCC | 280,082 | CttggCaatgatCtcAtCC | 9 | rs715-t | 3086 | 3104 | 12028 | 12046 | B372 |
| 280 | CTTGGCAATGATCTCATCC | 280,083 | CttgGcaAtgatctCatCC | 9 | rs715-t | 3086 | 3104 | 12028 | 12046 | B373 |
| 280 | CTTGGCAATGATCTCATCC | 280,084 | CttggcaatgAtCtcatCC | 9 | rs715-t | 3086 | 3104 | 12028 | 12046 | B374 |
| 280 | CTTGGCAATGATCTCATCC | 280,085 | CtTggcaatgatCTcaTCC | 9 | rs715-t | 3086 | 3104 | 12028 | 12046 | B375 |
| 280 | CTTGGCAATGATCTCATCC | 280,086 | CttgGcaatgatCtcAtCC | 9 | rs715-t | 3086 | 3104 | 12028 | 12046 | B376 |
| 280 | CTTGGCAATGATCTCATCC | 280,087 | CTTggcaatgatctcaTCC | 9 | rs715-t | 3086 | 3104 | 12028 | 12046 | B377 |
| 280 | CTTGGCAATGATCTCATCC | 280,088 | CTtggcaaTgaTcTcAtCC | 9 | rs715-t | 3086 | 3104 | 12028 | 12046 | B378 |
| 280 | CTTGGCAATGATCTCATCC | 280,089 | CTtggcaatgaTctcATCC | 9 | rs715-t | 3086 | 3104 | 12028 | 12046 | B379 |
| 280 | CTTGGCAATGATCTCATCC | 280,090 | CttgGcaatgAtCtcatCC | 9 | rs715-t | 3086 | 3104 | 12028 | 12046 | B380 |
| 280 | CTTGGCAATGATCTCATCC | 280,091 | CttggcAAtgatcTCatCC | 9 | rs715-t | 3086 | 3104 | 12028 | 12046 | B381 |
| 280 | CTTGGCAATGATCTCATCC | 280,092 | CTtgGcaatgatcTcaTCC | 9 | rs715-t | 3086 | 3104 | 12028 | 12046 | B382 |
| 280 | CTTGGCAATGATCTCATCC | 280,093 | CtTggcaatgatctCAtCC | 9 | rs715-t | 3086 | 3104 | 12028 | 12046 | B383 |
| 280 | CTTGGCAATGATCTCATCC | 280,094 | CTtggcaatGatCtcAtCC | 9 | rs715-t | 3086 | 3104 | 12028 | 12046 | B384 |
| 280 | CTTGGCAATGATCTCATCC | 280,095 | CttGgcaatgAtcTcAtCC | 9 | rs715-t | 3086 | 3104 | 12028 | 12046 | B385 |
| 280 | CTTGGCAATGATCTCATCC | 280,096 | CTtggcaatgaTctCaTCC | 9 | rs715-t | 3086 | 3104 | 12028 | 12046 | B386 |
| 280 | CTTGGCAATGATCTCATCC | 280,097 | CttggcaAtgATctCatCC | 9 | rs715-t | 3086 | 3104 | 12028 | 12046 | B387 |
| 280 | CTTGGCAATGATCTCATCC | 280,098 | CTtgGcaatgaTctCatCC | 9 | rs715-t | 3086 | 3104 | 12028 | 12046 | B388 |
| 280 | CTTGGCAATGATCTCATCC | 280,099 | CTtgGcaatgaTcTcAtCC | 9 | rs715-t | 3086 | 3104 | 12028 | 12046 | B389 |
| 280 | CTTGGCAATGATCTCATCC | 280,100 | CtTggcaatgATctCatCC | 9 | rs715-t | 3086 | 3104 | 12028 | 12046 | B390 |
| 280 | CTTGGCAATGATCTCATCC | 280,101 | CTtggcaatgatCtcATCC | 9 | rs715-t | 3086 | 3104 | 12028 | 12046 | B391 |
| 280 | CTTGGCAATGATCTCATCC | 280,102 | CTtggCaatgatcTcaTCC | 9 | rs715-t | 3086 | 3104 | 12028 | 12046 | B392 |
| 280 | CTTGGCAATGATCTCATCC | 280,103 | CttggcaAtGatCtcatCC | 9 | rs715-t | 3086 | 3104 | 12028 | 12046 | B393 |
| 280 | CTTGGCAATGATCTCATCC | 280,104 | CTtggCaatgatctcATCC | 9 | rs715-t | 3086 | 3104 | 12028 | 12046 | B394 |
| 280 | CTTGGCAATGATCTCATCC | 280,105 | CttGgcAatgatctCatCC | 9 | rs715-t | 3086 | 3104 | 12028 | 12046 | B395 |
| 280 | CTTGGCAATGATCTCATCC | 280,106 | CttggcAatgaTCtcatCC | 9 | rs715-t | 3086 | 3104 | 12028 | 12046 | B396 |
| 280 | CTTGGCAATGATCTCATCC | 280,107 | CttgGcAatgatCtcAtCC | 9 | rs715-t | 3086 | 3104 | 12028 | 12046 | B397 |
| 280 | CTTGGCAATGATCTCATCC | 280,108 | CttggcaAtgatCtcatCC | 9 | rs715-t | 3086 | 3104 | 12028 | 12046 | B398 |
| 280 | CTTGGCAATGATCTCATCC | 280,109 | CtTggcaatgAtcTcAtCC | 9 | rs715-t | 3086 | 3104 | 12028 | 12046 | B399 |
| 280 | CTTGGCAATGATCTCATCC | 280,110 | CTtggcaatgaTcTcaTCC | 9 | rs715-t | 3086 | 3104 | 12028 | 12046 | B400 |
| 280 | CTTGGCAATGATCTCATCC | 280,111 | CttggcAAtgatCtCatCC | 9 | rs715-t | 3086 | 3104 | 12028 | 12046 | B401 |
| 280 | CTTGGCAATGATCTCATCC | 280,112 | CTtggcaAtgatctCaTCC | 9 | rs715-t | 3086 | 3104 | 12028 | 12046 | B402 |
| 280 | CTTGGCAATGATCTCATCC | 280,113 | CttGgcaatgatctCAtCC | 9 | rs715-t | 3086 | 3104 | 12028 | 12046 | B403 |
| 280 | CTTGGCAATGATCTCATCC | 280,114 | CTTggcaatgatctcatCC | 9 | rs715-t | 3086 | 3104 | 12028 | 12046 | B404 |
| 280 | CTTGGCAATGATCTCATCC | 280,115 | CTtggcaaTgatcTcaTCC | 9 | rs715-t | 3086 | 3104 | 12028 | 12046 | B405 |
| 280 | CTTGGCAATGATCTCATCC | 280,116 | CttGgCaatgatCtcAtCC | 9 | rs715-t | 3086 | 3104 | 12028 | 12046 | B406 |
| 280 | CTTGGCAATGATCTCATCC | 280,117 | CttGgcaatgaTctCatCC | 9 | rs715-t | 3086 | 3104 | 12028 | 12046 | B407 |
| 280 | CTTGGCAATGATCTCATCC | 280,118 | CttGgCaatgatcTcAtCC | 9 | rs715-t | 3086 | 3104 | 12028 | 12046 | B408 |
| 280 | CTTGGCAATGATCTCATCC | 280,119 | CTtggcaaTgaTctCatCC | 9 | rs715-t | 3086 | 3104 | 12028 | 12046 | B409 |
| 280 | CTTGGCAATGATCTCATCC | 280,120 | CTtGgcaatgAtcTcAtCC | 9 | rs715-t | 3086 | 3104 | 12028 | 12046 | B410 |
| 280 | CTTGGCAATGATCTCATCC | 280,121 | CtTggcaatGaTctCatCC | 9 | rs715-t | 3086 | 3104 | 12028 | 12046 | B411 |
| 280 | CTTGGCAATGATCTCATCC | 280,122 | CTtggcaatGatctcATCC | 9 | rs715-t | 3086 | 3104 | 12028 | 12046 | B412 |
| 280 | CTTGGCAATGATCTCATCC | 280,123 | CttggcaatgatCtcatCC | 9 | rs715-t | 3086 | 3104 | 12028 | 12046 | B413 |
| 280 | CTTGGCAATGATCTCATCC | 280,124 | CTtgGcaatgatctCaTCC | 9 | rs715-t | 3086 | 3104 | 12028 | 12046 | B414 |
| 280 | CTTGGCAATGATCTCATCC | 280,125 | CttggcAatgatctCaTCC | 9 | rs715-t | 3086 | 3104 | 12028 | 12046 | B415 |
| 280 | CTTGGCAATGATCTCATCC | 280,126 | CTtGgcaatgatCtcAtCC | 9 | rs715-t | 3086 | 3104 | 12028 | 12046 | B416 |
| 280 | CTTGGCAATGATCTCATCC | 280,127 | CTtggcaatgatctcaTCC | 9 | rs715-t | 3086 | 3104 | 12028 | 12046 | B417 |
| 280 | CTTGGCAATGATCTCATCC | 280,128 | CttggcAAtgatCtcAtCC | 9 | rs715-t | 3086 | 3104 | 12028 | 12046 | B418 |
| 280 | CTTGGCAATGATCTCATCC | 280,129 | CTtggcaaTgatctCaTCC | 9 | rs715-t | 3086 | 3104 | 12028 | 12046 | B419 |
| 280 | CTTGGCAATGATCTCATCC | 280,130 | CttggcAatgAtCtcAtCC | 9 | rs715-t | 3086 | 3104 | 12028 | 12046 | B420 |
| 280 | CTTGGCAATGATCTCATCC | 280,131 | CttggcAATgatctCatCC | 9 | rs715-t | 3086 | 3104 | 12028 | 12046 | B421 |
| 280 | CTTGGCAATGATCTCATCC | 280,132 | CttggcAatGatCtcAtCC | 9 | rs715-t | 3086 | 3104 | 12028 | 12046 | B422 |
| 280 | CTTGGCAATGATCTCATCC | 280,133 | CTtgGcaatgatCtcATCC | 9 | rs715-t | 3086 | 3104 | 12028 | 12046 | B423 |
| 280 | CTTGGCAATGATCTCATCC | 280,134 | CTtGgcaatgatcTcaTCC | 9 | rs715-t | 3086 | 3104 | 12028 | 12046 | B424 |
| 280 | CTTGGCAATGATCTCATCC | 280,135 | CTtGgcaatgatctCaTCC | 9 | rs715-t | 3086 | 3104 | 12028 | 12046 | B425 |
| 280 | CTTGGCAATGATCTCATCC | 280,136 | CTtggcaatGatCtCatCC | 9 | rs715-t | 3086 | 3104 | 12028 | 12046 | B426 |
| 280 | CTTGGCAATGATCTCATCC | 280,137 | CtTggcaatGatCtcAtCC | 9 | rs715-t | 3086 | 3104 | 12028 | 12046 | B427 |
| 280 | CTTGGCAATGATCTCATCC | 280,138 | CTtgGcaatgatCtcAtCC | 9 | rs715-t | 3086 | 3104 | 12028 | 12046 | B428 |
| 280 | CTTGGCAATGATCTCATCC | 280,139 | CTtGgcaatgaTctCatCC | 9 | rs715-t | 3086 | 3104 | 12028 | 12046 | B429 |
| 280 | CTTGGCAATGATCTCATCC | 280,140 | CttggcaaTgaTcTcAtCC | 9 | rs715-t | 3086 | 3104 | 12028 | 12046 | B430 |
| 280 | CTTGGCAATGATCTCATCC | 280,141 | CttggcaAtgATcTcAtCC | 9 | rs715-t | 3086 | 3104 | 12028 | 12046 | B431 |
| 280 | CTTGGCAATGATCTCATCC | 280,142 | CTTggcaatgAtcTcAtCC | 9 | rs715-t | 3086 | 3104 | 12028 | 12046 | B432 |
| 280 | CTTGGCAATGATCTCATCC | 280,143 | CTtGgcaatgaTcTcAtCC | 9 | rs715-t | 3086 | 3104 | 12028 | 12046 | B433 |
| 280 | CTTGGCAATGATCTCATCC | 280,144 | CTtggcaatgAtcTcaTCC | 9 | rs715-t | 3086 | 3104 | 12028 | 12046 | B434 |
| 280 | CTTGGCAATGATCTCATCC | 280,145 | CttgGcaatgatctCatCC | 9 | rs715-t | 3086 | 3104 | 12028 | 12046 | B435 |
| 280 | CTTGGCAATGATCTCATCC | 280,146 | CTTggcaatgatctcATCC | 9 | rs715-t | 3086 | 3104 | 12028 | 12046 | B436 |
| 280 | CTTGGCAATGATCTCATCC | 280,147 | CttggcaatgatCtcaTCC | 9 | rs715-t | 3086 | 3104 | 12028 | 12046 | B437 |
| 280 | CTTGGCAATGATCTCATCC | 280,148 | CTtggCaatgatctCaTCC | 9 | rs715-t | 3086 | 3104 | 12028 | 12046 | B438 |
| 280 | CTTGGCAATGATCTCATCC | 280,149 | CTTggcaatgatctCaTCC | 9 | rs715-t | 3086 | 3104 | 12028 | 12046 | B439 |
| 280 | CTTGGCAATGATCTCATCC | 280,150 | CttgGcaatgatctCAtCC | 9 | rs715-t | 3086 | 3104 | 12028 | 12046 | B440 |
| 280 | CTTGGCAATGATCTCATCC | 280,151 | CTtggcaatgatCTcaTCC | 9 | rs715-t | 3086 | 3104 | 12028 | 12046 | B441 |
| 280 | CTTGGCAATGATCTCATCC | 280,152 | CttGgcAatgatCtcAtCC | 9 | rs715-t | 3086 | 3104 | 12028 | 12046 | B442 |
| 280 | CTTGGCAATGATCTCATCC | 280,153 | CttggcaatgAtCtCatCC | 9 | rs715-t | 3086 | 3104 | 12028 | 12046 | B443 |
| 280 | CTTGGCAATGATCTCATCC | 280,154 | CttGgcaAtgatctCatCC | 9 | rs715-t | 3086 | 3104 | 12028 | 12046 | B444 |
| 280 | CTTGGCAATGATCTCATCC | 280,155 | CTtGgcaatgatCTcaTCC | 9 | rs715-t | 3086 | 3104 | 12028 | 12046 | B445 |
| 280 | CTTGGCAATGATCTCATCC | 280,156 | CttgGcAAtgatctCatCC | 9 | rs715-t | 3086 | 3104 | 12028 | 12046 | B446 |
| 280 | CTTGGCAATGATCTCATCC | 280,157 | CTtggcAatgatctCaTCC | 9 | rs715-t | 3086 | 3104 | 12028 | 12046 | B447 |
| 280 | CTTGGCAATGATCTCATCC | 280,158 | CttggcAAtgatCtcatCC | 9 | rs715-t | 3086 | 3104 | 12028 | 12046 | B448 |
| 280 | CTTGGCAATGATCTCATCC | 280,159 | CttggCaatgatCtcatCC | 9 | rs715-t | 3086 | 3104 | 12028 | 12046 | B449 |
| 280 | CTTGGCAATGATCTCATCC | 280,160 | CTtggcAatgatcTcaTCC | 9 | rs715-t | 3086 | 3104 | 12028 | 12046 | B450 |
| 299 | TCGAGGTTAAATGGCTT | 299 | TCgaggttaaatgGCTT | | | | | 1049 | 1065 | S1 |
| 300 | ATCGAGGTTAAATGGCTT | 300 | ATCGaggttaaatggCTT | | | | | 1049 | 1066 | S2 |
| 301 | GGTCAGGGTAATGGTCA | 301 | GGtcagggtaatggtCA | | | | | 3374 | 3390 | S3 |
| 302 | AAACATGAAGGGGATGGA | 302 | AAACatgaaggggaTGGA | | | | | 3423 | 3440 | S4 |
| 303 | GGAAATGTTTCTGAAGGG | 303 | GGAAatgtttctgaagGG | | | | | 3533 | 3550 | SS |
| 304 | GAATGGGAAATGTTTCTG | 304 | GAATgggaaatgtttCTG | | | | | 3538 | 3555 | S6 |
| 305 | AAGTTGGTAGGGCTGGA | 305 | AAgttggtagggctgGA | | | | | 3557 | 3573 | S7 |
| 306 | AAAGTTGGTAGGGCTGG | 306 | AAAGttggtagggctGG | | | | | 3558 | 3574 | S8 |
| 307 | AAAAGTTGGTAGGGCTGG | 307 | AAaagttggtagggcTGG | | | | | 3558 | 3575 | S9 |
| 308 | AAAAGTTGGTAGGGCTG | 308 | AAaagttggtaggGCTG | | | | | 3559 | 3575 | S10 |
| 309 | GAAAAGTTGGTAGGGCTG | 309 | GAAaagttggtagggCTG | | | | | 3559 | 3576 | 511 |
| 310 | GAAAAGTTGGTAGGGCT | 310 | GAAaagttggtaggGCT | | | | | 3560 | 3576 | S12 |
| 311 | GGAAAAGTTGGTAGGGCT | 311 | GGaaaagttggtagggCT | | | | | 3560 | 3577 | S13 |
| 312 | AGAGACTTAAAGAGGAGA | 312 | AGAgacttaaagaggAGA | | | | | 4400 | 4417 | S14 |
| 313 | GGTTGTTGGTGATCAG | 313 | GGttgttggtgatCAG | | | 1035 | 1050 | 5064 | 5079 | S15 |
| 314 | ATGCATAATCGTAGGG | 314 | ATGcataatcgtAGGG | | | 1050 | 1065 | 5079 | 5094 | S16 |
| 315 | AAAGGATGTAAGATGCA | 315 | AAAGgatgtaagaTGCA | | | | | 5616 | 5632 | S17 |
| 316 | AAAGGATGTAAGATGC | 316 | AAAGgatgtaagATGC | | | | | 5617 | 5632 | S18 |
| 317 | ACAAAGGATGTAAGATG | 317 | ACAAaggatgtaaGATG | | | | | 5618 | 5634 | S19 |
| 318 | AACAAAGGATGTAAGATG | 318 | AACAaaggatgtaaGATG | | | | | 5618 | 5635 | S20 |
| 319 | CTATTTTGTCTATGGTGT | 319 | CTATtttgtctatggtGT | | | | | 7269 | 7286 | S21 |
| 320 | CTATTTTGTCTATGGTG | 320 | CTattttgtctatGGTG | | | | | 7270 | 7286 | S22 |
| 321 | CAGGTGGTTGTCAAACA | 321 | CAggtggttgtcaAACA | | | 1786 | 1802 | 7901 | 7917 | S23 |
| 322 | CATTGAAGTGGTGGGGTG | 322 | CAttgaagtggtggggTG | | | | | 8208 | 8225 | S24 |
| 323 | GATGGAGAGAATTCGAGA | 323 | GATGgagagaattcgaGA | | | | | 8749 | 8766 | S25 |
| 324 | CGTACAAAGTGGGGATG | 324 | CGtacaaagtgggGATG | | | 2127 | 2143 | 8894 | 8910 | S26 |
| 325 | ACGTACAAAGTGGGGATG | 325 | ACGtacaaagtggggATG | | | 2127 | 2144 | 8894 | 8911 | S27 |
| 326 | CGTACAAAGTGGGGAT | 326 | CGtacaaagtggGGAT | | | 2128 | 2143 | 8895 | 8910 | S28 |
| 327 | ACGTACAAAGTGGGGA | 327 | ACGtacaaagtggGGA | | | 2129 | 2144 | 8896 | 8911 | S29 |
| 328 | AACGTACAAAGTGGGG | 328 | AACGtacaaagtGGGG | | | 2130 | 2145 | 8897 | 8912 | S30 |
| 329 | AGTAGGAGGAGTCTGTGA | 329 | AGtaggaggagtctgtGA | | | | | 9605 | 9622 | S31 |
| 330 | AAGTAGGAGGAGTCTGTG | 330 | AAGtaggaggagtctgTG | | | | | 9606 | 9623 | S32 |
| 331 | GAAGTAGGAGGAGTCTGT | 331 | GAAgtaggaggagtctGT | | | | | 9607 | 9624 | S33 |
| 332 | GGAAGTAGGAGGAGTCTG | 332 | GGaagtaggaggagtcTG | | | | | 9608 | 9625 | S34 |
| 333 | GGAGGGGAAGAGTTTCAG | 333 | GGaggggaagagtttcAG | | | | | 11413 | 11430 | S35 |
| 334 | TCTTGCAGGTAGAGGGAA | 334 | TCttgcaggtagagggAA | | | | | 11503 | 11520 | S36 |
| 335 | GAGTGATAAGTGAGTCA | 335 | GAGTgataagtgagtCA | | | | | 12620 | 12636 | S37 |
| 336 | TTATTAGGGGACTGTGAG | 336 | TTAttaggggactgtGAG | | | | | 13243 | 13260 | S38 |
| 337 | GAAGGCTGTTATTTTCAT | 337 | GAAggctgttatttTCAT | | | | | 13794 | 13811 | S39 |
| 338 | GGAAGGCTGTTATTTTCA | 338 | GGaaggctgttatttTCA | | | | | 13795 | 13812 | S40 |
| 339 | AGGAGGGGATCTGAGAAC | 339 | AGgaggggatctgagAAC | | | | | 15987 | 16004 | S41 |
| 340 | AAGGAGGGGATCTGAGAA | 340 | AAGgaggggatctgaGAA | | | | | 15988 | 16005 | S42 |
| 341 | AGGCGTTCTTGAGTTTG | 341 | AGgcgttcttgagttTG | | | 4577 | 4593 | 18493 | 18509 | S43 |
| 342 | AAATGATCTGTACCAGG | 342 | AAAtgatctgtacCAGG | | | | | 21431 | 21447 | S44 |
| 343 | AAGTTCTGGAGGGTAGGG | 343 | AAgttctggagggtagGG | | | | | 22659 | 22676 | S45 |
| 344 | TGAGAGGGGTCTGATGG | 344 | TGagaggggtctgatGG | | | | | 22756 | 22772 | S46 |

*For Compounds, capital letters = LNA nucleosides, lower case letter = DNA nucleosides, optionally all internucleoside linkages are phosphorothioate.
In the examples, capital letters = beta-D-oxy-LNA nucleosides, LNA cytosines = 5 methyl cytosine LNA, lower case letters = DNA nucleosides, all internucleoside linkages between the nucleosides illustrated are phosphorothioate internucleoside linkages.

TABLE 6
Knock down of MYH7 RNA in 8820 and NH10 cells following treatment with 5 μM oligos.
RNA was measured using the QuantiGene assay. Both cells types are homozygous for
each SNP. Data presented at % mRNA compared to the level in PBS treated cells.

| | Perfect match to | Mismatch to | Perfect match to | Mismatch to | |
|---|---|---|---|---|---|
| | c-allele in | t-allele in | t-allele in | c-allele in | Compound ref. |
| CMP ID | 8820 cells | NH10 cells | NH10 cells | 8820 cells | used in |
| NO | (% PBS) | (% PBS) | (% PBS) | (% PBS) | examples |
| 11 | 112 | 113 | | | A1 |
| 12 | 102 | 73 | | | A2 |
| 13 | 102 | 264 | | | A3 |
| 14 | 118 | 161 | | | A4 |
| 15 | 132 | 87 | | | A5 |
| 16 | 117 | 125 | | | A6 |
| 17 | 124 | 92 | | | A7 |
| 18 | 136 | 87 | | | A8 |
| 19 | 106 | 218 | | | A9 |
| 20 | 100 | 115 | | | A10 |
| 21 | 121 | 121 | | | A11 |
| 22 | 100 | 81 | | | A12 |
| 23 | 101 | 246 | | | A13 |
| 24 | 121 | 122 | | | A14 |
| 25 | 115 | 90 | | | A15 |
| 26 | 99 | 98 | | | A16 |
| 27 | 99 | | | | A17 |
| 28 | 95 | 154 | | | A18 |
| 29 | 90 | 115 | | | A19 |
| 30 | 120 | 153 | | | A20 |
| 31 | 138 | 85 | | | A21 |
| 32 | 96 | 98 | | | A22 |
| 33 | 94 | 82 | | | A23 |
| 34 | 95 | 139 | | | A24 |
| 35 | 53 | 137 | | | A25 |
| 36 | 72 | 136 | | | A26 |
| 37 | 83 | 84 | | | A27 |
| 38 | 100 | 106 | | | A28 |
| 39 | 78 | 150 | | | A29 |
| 40 | 88 | 79 | | | A30 |
| 41 | 45 | 132 | | | A31 |
| 42 | 34 | 58 | | | A32 |
| 43 | 70 | 75 | | | A33 |
| 44 | 93 | 117 | | | A34 |
| 45 | 84 | 90 | | | A35 |
| 46 | 79 | 131 | | | A36 |
| 47 | 66 | 70 | | | A37 |
| 48 | 88 | 83 | | | A38 |
| 49 | 66 | 81 | | | A39 |
| 50 | 78 | | | | A40 |
| 51 | 36 | 59 | | | A41 |
| 52 | 60 | 71 | | | A42 |
| 53 | 90 | 68 | | | A43 |
| 54 | 51 | 45 | | | A44 |
| 55 | 55 | 70 | | | A45 |
| 56 | 89 | 121 | | | A46 |
| 57 | 84 | 100 | | | A47 |
| 58 | 60 | 92 | | | A48 |
| 59 | 34 | 49 | | | A49 |
| 60 | 40 | 106 | | | A50 |
| 61 | 31 | 42 | | | A51 |
| 62 | 39 | 80 | | | A52 |
| 63 | 40 | 55 | | | A53 |
| 64 | 32 | 52 | | | A54 |
| 65 | 48 | 87 | | | A55 |
| 66 | 93 | 141 | | | A56 |
| 67 | 100 | 125 | | | A57 |
| 68 | 104 | 118 | | | A58 |
| 69 | 114 | 331 | | | A59 |
| 70 | 106 | 97 | | | A60 |
| 71 | 90 | 133 | | | A61 |
| 72 | 92 | 178 | | | A62 |
| 73 | 93 | 145 | | | A63 |
| 74 | 102 | 212 | | | A64 |
| 75 | 105 | 356 | | | A65 |
| 76 | 68 | 130 | | | A66 |
| 77 | 118 | 135 | | | A67 |
| 78 | 101 | 118 | | | A68 |
| 79 | 121 | 107 | | | A69 |
| 80 | 123 | 77 | | | A70 |
| 81 | 55 | 128 | | | A71 |
| 82 | 110 | 137 | | | A72 |
| 83 | 124 | 169 | | | A73 |
| 84 | 104 | 153 | | | A74 |
| 85 | 104 | 189 | | | A75 |
| 86 | | | 52 | 64 | A76 |
| 87 | | | 101 | 139 | A77 |
| 88 | | | 20 | 59 | A78 |
| 89 | | | 49 | | A79 |
| 90 | | | 97 | 117 | A80 |
| 91 | | | 85 | 83 | A81 |
| 92 | | | 90 | 181 | A82 |
| 93 | | | 74 | 89 | A83 |
| 94 | | | 132 | 192 | A84 |
| 95 | | | 65 | 137 | A85 |
| 96 | | | 51 | 80 | A86 |
| 97 | | | 124 | 132 | A87 |
| 98 | | | 71 | | A88 |
| 99 | | | 100 | 139 | A89 |
| 100 | | | 103 | 196 | A90 |
| 101 | | | 129 | 146 | A91 |
| 102 | | | 106 | 154 | A92 |
| 103 | | | 55 | 94 | A93 |
| 104 | | | 97 | 171 | A94 |
| 105 | | | 99 | 131 | A95 |
| 106 | | | 112 | 128 | A96 |
| 107 | | | 51 | 84 | A97 |
| 108 | | | 77 | 128 | A98 |
| 109 | | | 66 | 116 | A99 |
| 110 | | | 53 | 133 | A100 |
| 111 | | | 148 | 123 | A101 |
| 112 | | | 122 | 159 | A102 |
| 113 | | | 36 | 56 | A103 |
| 114 | | | 95 | 164 | A104 |
| 115 | | | 95 | 126 | A105 |
| 116 | | | 57 | 140 | A106 |
| 117 | | | 82 | 128 | A107 |
| 118 | | | 36 | 89 | A108 |
| 119 | | | 33 | 60 | A109 |
| 120 | | | 59 | 73 | A110 |
| 121 | | | 37 | 109 | A111 |
| 122 | | | 118 | 136 | A112 |
| 123 | | | 85 | 131 | A113 |
| 124 | | | 100 | | A114 |
| 125 | | | 83 | 117 | A115 |
| 126 | | | 54 | 126 | A116 |
| 127 | | | 73 | 114 | A117 |
| 128 | | | 57 | 133 | A118 |
| 129 | | | 101 | 147 | A119 |
| 130 | | | 120 | 117 | A120 |
| 131 | | | 250 | 142 | A121 |
| 132 | | | 257 | 129 | A122 |
| 133 | | | 81 | 73 | A123 |
| 134 | | | 91 | 104 | A124 |
| 135 | | | 205 | 165 | A125 |
| 136 | | | 158 | 119 | A126 |
| 137 | | | 95 | 102 | A127 |
| 138 | | | 67 | 89 | A128 |
| 139 | | | 69 | 127 | A129 |
| 140 | | | 114 | 173 | A130 |
| 141 | | | 79 | 98 | A131 |
| 142 | | | 114 | 118 | A132 |
| 143 | | | 180 | 108 | A133 |
| 144 | | | 63 | 83 | A134 |
| 145 | | | 83 | 76 | A135 |
| 146 | | | 69 | 89 | A136 |
| 147 | | | 103 | 116 | A137 |
| 148 | | | 83 | 97 | A138 |
| 149 | | | 69 | 65 | A139 |
| 150 | | | 85 | 111 | A140 |
| 151 | | | 64 | 76 | A141 |
| 152 | | | 137 | 120 | A142 |
| 153 | | | 144 | 112 | A143 |
| 154 | | | 100 | 154 | A144 |
| 155 | | | 124 | 131 | A145 |
| 156 | | | 113 | 85 | A146 |
| 157 | | | 133 | 88 | A147 |
| 158 | | | 108 | 118 | A148 |
| 159 | | | 60 | 78 | A149 |
| 160 | | | 89 | 123 | A150 |
| 161 | | | 76 | 141 | A151 |
| 162 | | | 154 | 120 | A152 |
| 163 | | | 122 | 126 | A153 |
| 164 | | | 104 | 119 | A154 |
| 165 | | | 58 | 84 | A155 |
| 166 | | | 112 | 95 | A156 |
| 167 | | | 103 | 139 | A157 |
| 168 | | | 99 | 87 | A158 |
| 169 | | | 69 | 102 | A159 |
| 170 | | | 88 | 104 | A160 |
| 171 | | | 107 | 89 | A161 |
| 172 | | | 91 | 87 | A162 |
| 173 | | | 58 | 96 | A163 |
| 174 | | | 127 | 98 | A164 |
| 175 | | | 101 | 93 | A165 |
| 176 | | | 60 | 129 | A166 |
| 177 | | | 83 | 112 | A167 |
| 178 | | | 88 | 116 | A168 |
| 179 | | | 53 | 94 | A169 |
| 180 | | | 78 | 90 | A170 |
| 181 | | | 101 | 134 | A171 |
| 182 | | | 73 | 106 | A172 |
| 183 | | | 134 | 106 | A173 |
| 184 | | | 90 | 117 | A174 |
| 185 | | | 67 | 100 | A175 |
| 186 | | | 123 | 142 | A176 |
| 187 | | | 125 | 114 | A177 |
| 188 | | | 88 | 130 | A178 |
| 189 | | | 95 | 119 | A179 |
| 190 | | | 169 | 130 | A180 |
| 191 | | | 50 | 88 | A181 |
| 192 | | | 76 | 89 | A182 |
| 193 | 100 | 168 | | | A183 |
| 194 | 95 | 139 | | | A184 |
| 195 | 104 | 125 | | | A185 |
| 196 | 98 | 178 | | | A186 |
| 197 | 58 | 165 | | | A187 |
| 198 | 120 | 136 | | | A188 |
| 199 | 86 | 91 | | | A189 |
| 200 | 50 | 131 | | | A190 |
| 201 | 103 | 203 | | | A191 |
| 202 | 82 | 192 | | | A192 |
| 203 | 123 | 133 | | | A193 |
| 204 | 28 | 127 | | | A194 |
| 205 | 83 | 125 | | | A195 |
| 206 | 94 | 143 | | | A196 |
| 207 | 36 | 85 | | | A197 |
| 208 | 47 | 94 | | | A198 |
| 209 | 53 | 93 | | | A199 |
| 210 | 72 | 164 | | | A200 |
| 211 | 35 | 82 | | | A201 |
| 212 | 38 | 68 | | | A202 |
| 213 | 26 | 53 | | | A203 |
| 214 | 40 | 131 | | | A204 |
| 215 | 38 | 49 | | | A205 |
| 216 | 40 | 112 | | | A206 |
| 217 | 52 | 93 | | | A207 |
| 218 | 81 | 191 | | | A208 |
| 219 | 45 | 84 | | | A209 |
| 220 | 45 | 130 | | | A210 |
| 221 | 70 | 175 | | | A211 |
| 222 | 89 | 209 | | | A212 |
| 223 | 32 | 66 | | | A213 |
| 224 | 23 | 94 | | | A214 |
| 225 | 57 | 132 | | | A215 |
| 226 | 120 | 129 | | | A216 |
| 227 | 13 | 43 | | | A217 |
| 228 | 34 | 112 | | | A218 |
| 229 | 114 | 92 | | | A219 |
| 230 | 124 | 159 | | | A220 |
| 231 | 102 | 134 | | | A221 |
| 232 | 120 | 101 | | | A222 |
| 233 | 87 | 91 | | | A223 |
| 234 | 107 | 151 | | | A224 |
| 235 | 132 | 154 | | | A225 |
| 236 | 49 | 96 | | | A226 |
| 237 | 78 | 175 | | | A227 |
| 238 | 93 | 177 | | | A228 |
| 239 | 44 | 133 | | | A229 |
| 240 | 46 | 88 | | | A230 |
| 241 | 50 | 88 | | | A231 |
| 242 | 41 | 134 | | | A232 |
| 243 | 36 | 96 | | | A233 |
| 244 | 34 | 65 | | | A234 |
| 245 | 66 | 156 | | | A235 |
| 246 | 49 | 160 | | | A236 |
| 247 | 54 | 116 | | | A237 |
| 248 | 80 | 151 | | | A238 |
| 249 | 80 | 125 | | | A239 |
| 250 | 54 | 89 | | | A240 |
| 251 | 81 | 130 | | | A241 |
| 252 | | | 63 | 76 | A242 |
| 253 | | | 138 | 99 | A243 |
| 254 | | | 81 | 80 | A244 |
| 255 | | | 89 | 70 | A245 |
| 256 | | | 43 | 40 | A246 |
| 257 | | | 120 | 101 | A247 |
| 258 | | | 93 | 100 | A248 |
| 259 | | | 60 | 76 | A249 |
| 260 | | | 55 | 53 | A250 |
| 261 | | | 55 | 59 | A251 |
| 262 | | | 122 | 104 | A252 |
| 263 | | | 86 | 71 | A253 |
| 264 | | | 82 | 82 | A254 |
| 265 | | | 57 | 52 | A255 |
| 266 | | | 94 | 108 | A256 |
| 267 | 43 | 117 | | | A257 |
| 268 | 109 | 171 | | | A258 |
| 269 | 42 | 151 | | | A259 |
| 270 | 61 | 74 | | | A260 |
| 271 | 47 | 164 | | | A261 |
| 272 | 57 | 103 | | | A262 |
| 273 | 67 | 92 | | | A263 |
| 274 | 51 | 128 | | | A264 |
| 275 | 80 | 146 | | | A265 |
| 276 | 60 | 97 | | | A266 |
| 277 | 48 | 125 | | | A267 |
| 278 | 47 | 135 | | | A268 |
| 279 | 29 | 86 | | | A269 |
| 280 | 34 | 97 | | | A270 |
| 281 | 66 | 151 | | | A271 |
| 282 | 70 | 102 | | | A272 |
| 283 | 55 | 202 | | | A273 |
| 284 | 58 | 116 | | | A274 |
| 285 | 67 | 102 | | | A275 |
| 286 | 59 | 140 | | | A276 |
| 287 | 79 | | | | A277 |
| 288 | 79 | 173 | | | A278 |
| 289 | 59 | 134 | | | A279 |
| 290 | 80 | 122 | | | A280 |
| 291 | 72 | 133 | | | A281 |
| 292 | 73 | 78 | | | A282 |
| 293 | 51 | 141 | | | A283 |
| 294 | 45 | 116 | | | A284 |
| 295 | 33 | 73 | | | A285 |
| 296 | 41 | 82 | | | A286 |
| 297 | 47 | 136 | | | A287 |
| 298 | 58 | 142 | | | A288 |
| 315 | 15 | 16 | 17 | 13 | S17 |

TABLE 7
Knock down of MYH7 RNA in CC-2580 cells following 6 days treatment
with 5 μM oligonucleotide. RNA was measured using the ddPCR
assay. The cell line is heterozygous for the Rs715 SNP. Data presented
at % mRNA compared to the level in PBS treated cells.

| | Perfect match to | Mismatch to | Perfect match to | Mismatch to | |
|---|---|---|---|---|---|
| | c-allele in | t-allele in | t-allele in | c-allele in | Compound ref. |
| CMP ID | CC-2580 cells | CC-2580 cells | CC-2580 cells | CC-2580 cells | used in |
| NO | (% PBS) | (% PBS) | (% PBS) | (% PBS) | examples |
| 260.1 | 30 | 54 | | | B1 |
| 260.2 | 96 | 107 | | | B2 |
| 259.1 | 98 | 100 | | | B3 |
| 259.2 | 66 | 111 | | | B4 |
| 259.3 | 26 | 61 | | | B5 |
| 259.4 | 47 | 91 | | | B6 |
| 259.5 | 110 | 170 | | | B7 |
| 259.6 | 74 | 112 | | | B8 |
| 260.3 | 86 | 111 | | | B9 |
| 259.7 | 60 | 107 | | | B10 |
| 259.8 | 74 | 93 | | | B11 |
| 260.4 | 52 | 79 | | | B12 |
| 259.9 | 75 | 100 | | | B13 |
| 259.1 | 74 | 130 | | | B14 |
| 259.11 | 80 | 81 | | | B15 |
| 259.12 | 42 | 73 | | | B16 |
| 259.13 | 70 | 68 | | | B17 |
| 259.14 | 60 | 78 | | | B18 |
| 259.15 | 132 | 142 | | | B19 |
| 259.16 | 99 | 108 | | | B20 |
| 260.5 | 28 | 51 | | | B21 |
| 259.17 | 89 | 116 | | | B22 |
| 259.18 | 60 | 108 | | | B23 |
| 259.19 | 68 | 83 | | | B24 |
| 259.2 | 51 | 70 | | | B25 |
| 259.21 | 71 | 140 | | | B26 |
| 260.6 | 68 | 125 | | | B27 |
| 259.22 | 91 | 110 | | | B28 |
| 260.7 | 78 | 104 | | | B29 |
| 260.8 | 49 | 80 | | | B30 |
| 259.23 | 53 | 70 | | | B31 |
| 259.24 | 41 | 84 | | | B32 |
| 259.25 | 75 | 84 | | | B33 |
| 259.26 | 65 | 82 | | | B34 |
| 260.9 | 130 | 141 | | | B35 |
| 259.27 | 61 | 70 | | | B36 |
| 260.1 | 49 | 63 | | | B37 |
| 259.28 | 40 | 63 | | | B38 |
| 259.29 | 51 | 78 | | | B39 |
| 259.3 | 61 | 91 | | | B40 |
| 260.11 | 78 | 112 | | | B41 |
| 260.12 | 39 | 75 | | | B42 |
| 259.31 | 33 | 55 | | | B43 |
| 260.13 | 30 | 72 | | | B44 |
| 260.14 | 69 | 84 | | | B45 |
| 259.32 | 79 | 97 | | | B46 |
| 259.33 | 59 | 101 | | | B47 |
| 260.15 | 52 | 54 | | | B48 |
| 259.34 | 79 | 134 | | | B49 |
| 260.16 | 82 | 98 | | | B50 |
| 260.17 | 61 | 73 | | | B51 |
| 260.18 | 82 | 114 | | | B52 |
| 260.19 | 75 | 88 | | | B53 |
| 260.2 | 125 | 142 | | | B54 |
| 260.21 | 20 | 50 | | | B55 |
| 260.22 | 24 | 44 | | | B56 |
| 260.23 | 133 | 160 | | | B57 |
| 259.35 | 101 | 107 | | | B58 |
| 259.36 | 64 | 81 | | | B59 |
| 259.37 | 105 | 139 | | | B60 |
| 259.38 | 103 | 143 | | | B61 |
| 259.39 | 36 | 57 | | | B62 |
| 260.24 | 79 | 79 | | | B63 |
| 259.4 | 86 | 92 | | | B64 |
| 259.41 | 93 | 109 | | | B65 |
| 259.42 | 56 | 84 | | | B66 |
| 259.43 | 60 | 80 | | | B67 |
| 259.44 | 91 | 98 | | | B68 |
| 260.25 | 83 | 129 | | | B69 |
| 259.45 | 127 | 145 | | | B70 |
| 259.46 | 72 | 95 | | | B71 |
| 259.47 | 59 | 88 | | | B72 |
| 260.26 | 70 | 74 | | | B73 |
| 259.48 | 66 | 83 | | | B74 |
| 260.27 | 95 | 135 | | | B75 |
| 259.49 | 51 | 89 | | | B76 |
| 259.5 | 93 | 123 | | | B77 |
| 259.51 | 79 | 96 | | | B78 |
| 259.52 | 73 | 99 | | | B79 |
| 259.53 | 91 | 116 | | | B80 |
| 259.54 | 60 | 114 | | | B81 |
| 260.28 | 34 | 67 | | | B82 |
| 260.29 | 68 | 101 | | | B83 |
| 259.55 | 20 | 67 | | | B84 |
| 260.3 | 22 | 53 | | | B85 |
| 259.56 | 112 | 160 | | | B86 |
| 260.31 | 32 | 48 | | | B87 |
| 260.32 | 70 | 120 | | | B88 |
| 259.57 | 92 | 102 | | | B89 |
| 259.58 | 62 | 80 | | | B90 |
| 260.33 | 41 | 60 | | | B91 |
| 259.59 | 81 | 110 | | | B92 |
| 259.6 | 76 | 114 | | | B93 |
| 259.61 | 95 | 108 | | | B94 |
| 259.62 | 69 | 93 | | | B95 |
| 259.63 | 55 | 114 | | | B96 |
| 260.34 | 65 | 84 | | | B97 |
| 260.35 | 56 | 84 | | | B98 |
| 260.36 | 36 | 45 | | | B99 |
| 259.64 | 81 | 95 | | | B100 |
| 259.65 | 71 | 107 | | | B101 |
| 259.66 | 55 | 69 | | | B102 |
| 259.67 | 87 | 97 | | | B103 |
| 259.68 | 35 | 65 | | | B104 |
| 259.69 | 85 | 109 | | | B105 |
| 259.7 | 41 | 46 | | | B106 |
| 259.71 | 88 | 97 | | | B107 |
| 259.72 | 54 | 72 | | | B108 |
| 260.37 | 67 | 97 | | | B109 |
| 260.38 | 84 | 102 | | | B110 |
| 260.39 | 64 | 89 | | | B111 |
| 259.73 | 79 | 122 | | | B112 |
| 260.4 | 104 | 112 | | | B113 |
| 259.74 | 57 | 101 | | | B114 |
| 260.41 | 79 | 99 | | | B115 |
| 259.75 | 63 | 67 | | | B116 |
| 260.42 | 58 | 97 | | | B117 |
| 259.76 | 57 | 86 | | | B118 |
| 259.77 | 121 | 150 | | | B119 |
| 259.78 | 72 | 94 | | | B120 |
| 259.79 | 91 | 110 | | | B121 |
| 259.8 | 56 | 90 | | | B122 |
| 260.43 | 64 | 81 | | | B123 |
| 259.81 | 25 | 41 | | | B124 |
| 259.82 | 63 | 98 | | | B125 |
| 260.44 | 57 | 97 | | | B126 |
| 260.45 | 64 | 99 | | | B127 |
| 259.83 | 48 | 80 | | | B128 |
| 260.46 | 65 | 92 | | | B129 |
| 259.84 | 117 | 115 | | | B130 |
| 259.85 | 85 | 114 | | | B131 |
| 259.86 | 68 | 107 | | | B132 |
| 259.87 | 113 | 147 | | | B133 |
| 259.88 | 100 | 124 | | | B134 |
| 259.89 | 58 | 88 | | | B135 |
| 259.9 | 63 | 86 | | | B136 |
| 259.91 | 106 | 99 | | | B137 |
| 259.92 | 79 | 125 | | | B138 |
| 260.47 | 92 | 129 | | | B139 |
| 259.93 | 116 | 152 | | | B140 |
| 260.48 | 33 | 45 | | | B141 |
| 260.49 | 56 | 93 | | | B142 |
| 259.94 | 110 | 151 | | | B143 |
| 259.95 | 80 | 141 | | | B144 |
| 259.96 | 84 | 86 | | | B145 |
| 259.97 | 71 | 77 | | | B146 |
| 259.98 | 33 | 64 | | | B147 |
| 260.5 | 75 | 96 | | | B148 |
| 259.99 | 35 | 55 | | | B149 |
| 260.51 | 37 | 80 | | | B150 |
| 259.1 | 65 | 93 | | | B151 |
| 260.52 | 67 | 82 | | | B152 |
| 259.101 | 40 | 87 | | | B153 |
| 259.102 | 57 | 91 | | | B154 |
| 259.103 | 52 | 74 | | | B155 |
| 260.53 | 53 | 77 | | | B156 |
| 259.104 | 50 | 90 | | | B157 |
| 260.54 | 86 | 100 | | | B158 |
| 259.105 | 83 | 125 | | | B159 |
| 259.106 | 101 | 110 | | | B160 |
| 259.107 | 59 | 137 | | | B161 |
| 259.108 | 66 | 84 | | | B162 |
| 259.109 | 58 | 91 | | | B163 |
| 260.55 | 57 | 55 | | | B164 |
| 260.56 | 64 | 66 | | | B165 |
| 259.11 | 82 | 95 | | | B166 |
| 259.111 | 59 | 89 | | | B167 |
| 259.112 | 31 | 68 | | | B168 |
| 259.113 | 78 | 86 | | | B169 |
| 259.114 | 96 | 136 | | | B170 |
| 260.57 | 83 | 97 | | | B171 |
| 259.115 | 34 | 69 | | | B172 |
| 259.116 | 115 | 142 | | | B173 |
| 259.117 | 93 | 116 | | | B174 |
| 260.58 | 36 | 60 | | | B175 |
| 259.118 | 70 | 75 | | | B176 |
| 259.119 | 91 | 133 | | | B177 |
| 259.12 | 73 | 112 | | | B178 |
| 259.121 | 58 | 91 | | | B179 |
| 260.59 | 58 | 89 | | | B180 |
| 260.6 | 69 | 60 | | | B181 |
| 260.61 | 69 | 91 | | | B182 |
| 259.122 | 144 | 136 | | | B183 |
| 259.123 | 70 | 108 | | | B184 |
| 260.62 | 78 | 121 | | | B185 |
| 259.124 | 130 | 130 | | | B186 |
| 260.63 | 72 | 100 | | | B187 |
| 259.125 | 95 | 112 | | | B188 |
| 259.126 | 86 | 82 | | | B189 |
| 260.64 | 56 | 89 | | | B190 |
| 259.127 | 101 | 124 | | | B191 |
| 259.128 | 92 | 128 | | | B192 |
| 260.65 | 65 | 83 | | | B193 |
| 259.129 | 52 | 71 | | | B194 |
| 260.66 | 72 | 124 | | | B195 |
| 260.67 | 69 | 103 | | | B196 |
| 259.13 | 82 | 97 | | | B197 |
| 260.68 | 77 | 106 | | | B198 |
| 259.131 | 62 | 78 | | | B199 |
| 260.69 | 50 | 68 | | | B200 |
| 259.132 | 147 | 175 | | | B201 |
| 259.133 | 58 | 96 | | | B202 |
| 259.134 | 46 | 82 | | | B203 |
| 259.135 | 73 | 98 | | | B204 |
| 260.7 | 86 | 92 | | | B205 |
| 259.136 | 62 | 71 | | | B206 |
| 259.137 | 62 | 107 | | | B207 |
| 259.138 | 88 | 104 | | | B208 |
| 260.71 | 65 | 84 | | | B209 |
| 260.72 | 63 | 89 | | | B210 |
| 260.73 | 81 | 119 | | | B211 |
| 260.74 | 64 | 66 | | | B212 |
| 259.139 | 97 | 101 | | | B213 |
| 259.14 | 74 | 95 | | | B214 |
| 259.141 | 83 | 97 | | | B215 |
| 259.142 | 69 | 97 | | | B216 |
| 259.143 | 117 | 129 | | | B217 |
| 259.144 | 79 | 87 | | | B218 |
| 259.145 | 96 | 121 | | | B219 |
| 260.75 | 97 | 115 | | | B220 |
| 260.76 | 58 | 85 | | | B221 |
| 259.146 | 69 | 83 | | | B222 |
| 259.147 | 61 | 115 | | | B223 |
| 259.148 | 61 | 73 | | | B224 |
| 259.149 | 72 | 96 | | | B225 |
| 259.15 | 62 | 66 | | | B226 |
| 260.77 | 53 | 70 | | | B227 |
| 259.151 | 99 | 101 | | | B228 |
| 260.78 | 73 | 104 | | | B229 |
| 259.152 | 104 | 111 | | | B230 |
| 259.153 | 76 | 87 | | | B231 |
| 259.154 | 72 | 97 | | | B232 |
| 260.79 | 41 | 79 | | | B233 |
| 259.155 | 68 | 98 | | | B234 |
| 260.8 | 63 | 94 | | | B235 |
| 260.81 | 100 | 98 | | | B236 |
| 259.156 | 79 | 71 | | | B237 |
| 259.157 | 96 | 120 | | | B238 |
| 260.82 | 54 | 55 | | | B239 |
| 259.158 | 80 | 85 | | | B240 |
| 259.159 | 77 | 84 | | | B241 |
| 259.16 | 63 | 65 | | | B242 |
| 259.161 | 83 | 102 | | | B243 |
| 260.83 | 56 | 82 | | | B244 |
| 259.162 | 91 | 115 | | | B245 |
| 259.163 | 82 | 97 | | | B246 |
| 260.84 | 95 | 116 | | | B247 |
| 260.85 | 66 | 101 | | | B248 |
| 259.164 | 71 | 97 | | | B249 |
| 259.165 | 89 | 85 | | | B250 |
| 259.166 | 102 | 112 | | | B251 |
| 259.167 | 34 | 54 | | | B252 |
| 259.168 | 53 | 80 | | | B253 |
| 260.86 | 38 | 70 | | | B254 |
| 259.169 | 85 | 95 | | | B255 |
| 259.17 | 72 | 102 | | | B256 |
| 259.171 | 71 | 95 | | | B257 |
| 260.87 | 65 | 76 | | | B258 |
| 260.88 | 69 | 83 | | | B259 |
| 260.89 | 60 | 67 | | | B260 |
| 259.172 | 53 | 75 | | | B261 |
| 260.9 | 75 | 113 | | | B262 |
| 260.91 | 88 | 105 | | | B263 |
| 259.173 | 66 | 104 | | | B264 |
| 260.92 | 137 | 145 | | | B265 |
| 259.174 | 63 | 114 | | | B266 |
| 259.175 | 36 | 54 | | | B267 |
| 260.93 | 74 | 102 | | | B268 |
| 259.176 | 117 | 143 | | | B269 |
| 259.177 | 85 | 88 | | | B270 |
| 259.178 | 29 | 62 | | | B271 |
| 260.94 | 44 | 67 | | | B272 |
| 260.95 | 63 | 68 | | | B273 |
| 260.96 | 42 | 54 | | | B274 |
| 259.179 | 85 | 113 | | | B275 |
| 260.97 | 100 | 145 | | | B276 |
| 259.18 | 50 | 92 | | | B277 |
| 259.181 | 82 | 105 | | | B278 |
| 260.98 | 58 | 64 | | | B279 |
| 260.99 | 89 | 135 | | | B280 |
| 259.182 | 95 | 121 | | | B281 |
| 259.183 | 47 | 88 | | | B282 |
| 259.184 | 78 | 93 | | | B283 |
| 259.185 | 60 | 69 | | | B284 |
| 259.186 | 73 | 96 | | | B285 |
| 259.187 | 77 | 101 | | | B286 |
| 259.188 | 68 | 97 | | | B287 |
| 259.189 | 77 | 98 | | | B288 |
| 260.1 | 76 | 80 | | | B289 |
| 260.101 | 92 | 106 | | | B290 |
| 280.1 | | | 62 | 92 | B291 |
| 280.2 | | | 75 | 111 | B292 |
| 280.3 | | | 78 | 96 | B293 |
| 280.4 | | | 40 | 55 | B294 |
| 280.5 | | | 28 | 71 | B295 |
| 280.6 | | | 32 | 67 | B296 |
| 280.7 | | | 66 | 90 | B297 |
| 280.8 | | | 20 | 36 | B298 |
| 280.9 | | | 27 | 84 | B299 |
| 280.1 | | | 58 | 103 | B300 |
| 280.11 | | | 45 | 89 | B301 |
| 280.12 | | | 73 | 113 | B302 |
| 280.13 | | | 33 | 57 | B303 |
| 280.14 | | | 37 | 65 | B304 |
| 280.15 | | | 51 | 85 | B305 |
| 280.16 | | | 47 | 97 | B306 |
| 280.17 | | | 42 | 79 | B307 |
| 280.18 | | | 29 | 38 | B308 |
| 280.19 | | | 62 | 115 | B309 |
| 280.2 | | | 74 | 95 | B310 |
| 280.21 | | | 66 | 107 | B311 |
| 280.22 | | | 93 | 102 | B312 |
| 280.23 | | | 46 | 69 | B313 |
| 280.24 | | | 63 | 124 | B314 |
| 280.25 | | | 52 | 82 | B315 |
| 280.26 | | | 55 | 84 | B316 |
| 280.27 | | | 42 | 81 | B317 |
| 280.28 | | | 46 | 91 | B318 |
| 280.29 | | | 46 | 67 | B319 |
| 280.3 | | | 32 | 57 | B320 |
| 280.31 | | | 47 | 96 | B321 |
| 280.32 | | | 39 | 50 | B322 |
| 280.33 | | | 45 | 84 | B323 |
| 280.34 | | | 29 | 65 | B324 |
| 280.35 | | | 76 | 71 | B325 |
| 280.36 | | | 66 | 83 | B326 |
| 280.37 | | | 89 | 118 | B327 |
| 280.38 | | | 114 | 143 | B328 |
| 280.39 | | | 107 | 117 | B329 |
| 280.4 | | | 56 | 73 | B330 |
| 280.41 | | | 57 | 76 | B331 |
| 280.42 | | | 31 | 64 | B332 |
| 280.43 | | | 52 | 85 | B333 |
| 280.44 | | | 17 | 51 | B334 |
| 280.45 | | | 37 | 63 | B335 |
| 280.46 | | | 81 | 109 | B336 |
| 280.47 | | | 55 | 71 | B337 |
| 280.48 | | | 49 | 83 | B338 |
| 280.49 | | | 31 | 55 | B339 |
| 280.5 | | | 55 | 69 | B340 |
| 280.51 | | | 51 | 83 | B341 |
| 280.52 | | | 19 | 40 | B342 |
| 280.53 | | | 68 | 96 | B343 |
| 280.54 | | | 70 | 78 | B344 |
| 280.55 | | | 42 | 85 | B345 |
| 280.56 | | | 102 | 145 | B346 |
| 280.57 | | | 35 | 62 | B347 |
| 280.58 | | | 51 | 91 | B348 |
| 280.59 | | | 104 | 129 | B349 |
| 280.6 | | | 63 | 93 | B350 |
| 280.61 | | | 59 | 102 | B351 |
| 280.62 | | | 44 | 61 | B352 |
| 280.63 | | | 58 | 86 | B353 |
| 280.64 | | | 62 | 108 | B354 |
| 280.65 | | | 17 | 45 | B355 |
| 280.66 | | | 70 | 105 | B356 |
| 280.67 | | | 91 | 119 | B357 |
| 280.68 | | | 50 | 66 | B358 |
| 280.69 | | | 27 | 42 | B359 |
| 280.7 | | | 34 | 64 | B360 |
| 280.71 | | | 43 | 79 | B361 |
| 280.72 | | | 103 | 110 | B362 |
| 280.73 | | | 72 | 101 | B363 |
| 280.74 | | | 60 | 99 | B364 |
| 280.75 | | | 56 | 74 | B365 |
| 280.76 | | | 84 | 104 | B366 |
| 280.77 | | | 114 | 132 | B367 |
| 280.78 | | | 103 | 125 | B368 |
| 280.79 | | | 94 | 116 | B369 |
| 280.8 | | | 41 | 61 | B370 |
| 280.81 | | | 51 | 83 | B371 |
| 280.82 | | | 94 | 133 | B372 |
| 280.83 | | | 55 | 119 | B373 |
| 280.84 | | | 55 | 78 | B374 |
| 280.85 | | | 29 | 56 | B375 |
| 280.86 | | | 78 | 97 | B376 |
| 280.87 | | | 29 | 54 | B377 |
| 280.88 | | | 65 | 95 | B378 |
| 280.89 | | | 48 | 78 | B379 |
| 280.9 | | | 88 | 99 | B380 |
| 280.91 | | | 29 | 74 | B381 |
| 280.92 | | | 47 | 72 | B382 |
| 280.93 | | | 22 | 54 | B383 |
| 280.94 | | | 41 | 64 | B384 |
| 280.95 | | | 86 | 94 | B385 |
| 280.96 | | | 32 | 62 | B386 |
| 280.97 | | | 66 | 83 | B387 |
| 280.98 | | | 154 | 116 | B388 |
| 280.99 | | | 125 | 143 | B389 |
| 280.1 | | | 36 | 53 | B390 |
| 280.101 | | | 23 | 49 | B391 |
| 280.102 | | | 44 | 83 | B392 |
| 280.103 | | | 40 | 68 | B393 |
| 280.104 | | | 16 | 43 | B394 |
| 280.105 | | | 43 | 80 | B395 |
| 280.106 | | | 64 | 98 | B396 |
| 280.107 | | | 71 | 114 | B397 |
| 280.108 | | | 59 | 81 | B398 |
| 280.109 | | | 57 | 86 | B399 |
| 280.11 | | | 48 | 66 | B400 |
| 280.111 | | | 33 | 48 | B401 |
| 280.112 | | | 30 | 64 | B402 |
| 280.113 | | | 19 | 56 | B403 |
| 280.114 | | | 35 | 64 | B404 |
| 280.115 | | | 38 | 70 | B405 |
| 280.116 | | | 121 | 162 | B406 |
| 280.117 | | | 40 | 63 | B407 |
| 280.118 | | | 97 | 128 | B408 |
| 280.119 | | | 52 | 82 | B409 |
| 280.12 | | | 99 | 99 | B410 |
| 280.121 | | | 52 | 67 | B411 |
| 280.122 | | | 51 | 97 | B412 |
| 280.123 | | | 70 | 124 | B413 |
| 280.124 | | | 38 | 74 | B414 |
| 280.125 | | | 25 | 61 | B415 |
| 280.126 | | | 62 | 77 | B416 |
| 280.127 | | | 84 | 135 | B417 |
| 280.128 | | | 76 | 100 | B418 |
| 280.129 | | | 32 | 59 | B419 |
| 280.13 | | | 69 | 78 | B420 |
| 280.131 | | | 71 | 80 | B421 |
| 280.132 | | | 106 | 121 | B422 |
| 280.133 | | | 44 | 89 | B423 |
| 280.134 | | | 36 | 62 | B424 |
| 280.135 | | | 31 | 53 | B425 |
| 280.136 | | | 41 | 66 | B426 |
| 280.137 | | | 67 | 105 | B427 |
| 280.138 | | | 74 | 102 | B428 |
| 280.139 | | | 42 | 55 | B429 |
| 280.14 | | | 63 | 88 | B430 |
| 280.141 | | | 82 | 107 | B431 |
| 280.142 | | | 40 | 65 | B432 |
| 280.143 | | | 61 | 74 | B433 |
| 280.144 | | | 34 | 69 | B434 |
| 280.145 | | | 36 | 63 | B435 |
| 280.146 | | | 11 | 34 | B436 |
| 280.147 | | | 39 | 88 | B437 |
| 280.148 | | | 39 | 77 | B438 |
| 280.149 | | | 15 | 26 | B439 |
| 280.15 | | | 33 | 101 | B440 |
| 280.151 | | | 26 | 51 | B441 |
| 280.152 | | | 54 | 87 | B442 |
| 280.153 | | | 42 | 76 | B443 |
| 280.154 | | | 68 | 122 | B444 |
| 280.155 | | | 42 | 79 | B445 |
| 280.156 | | | 83 | 118 | B446 |
| 280.157 | | | 20 | 53 | B447 |
| 280.158 | | | 43 | 71 | B448 |
| 280.159 | | | 57 | 86 | B449 |
| 280.16 | | | 47 | 96 | B450 |
| 280 | | | 30 | 61 | A270 |
| 259 | 23 | 58 | | | A249 |
| 260 | 31 | 50 | | | A250 |
| 315 | 12 | 11 | 11 | 12 | S17 |

TABLE 8
Knock down of MYH7 RNA in CC-2580 cells following 6 days treatment with various concentration of
oligonucleotide. Allele specific RNA was measured using the ddPCR assay. EC50 values are calculated
for each allele and the selectivity was calculated as the ratio between the two EC50 values.

| | EC50 for | EC50 for | | EC50 for | EC50 for | | |
|---|---|---|---|---|---|---|---|
| | perfect match | mismatch to | Selectivity | perfect match | mismatch to | Selectivity | |
| | to c-allele in | t-allele in | between c- and | to t-allele in | c-allele in | between t- and | Compound |
| CMP ID | CC-2580 cells | CC-2580 cells | t-allele in | CC-2580 cells | CC-2580 cells | c-allele in | ref. used in |
| NO | (uM) | (uM) | CC-2580 cells | (uM) | (uM) | CC-2580 cells | examples |
| 259 | 0.18 | 3.67 | 20.9 | | | | A249 |
| 260 | 0.13 | 0.35 | 2.6 | | | | A250 |
| 280 | | | | 0.12 | 0.86 | 7.2 | A270 |
| 280.91 | | | | 0.26 | 5.00 | 19.2 | B381 |
| 280.93 | | | | 0.36 | 1.95 | 5.4 | B383 |
| 280.113 | | | | 0.13 | 1.01 | 7.8 | B403 |
| 280.15 | | | | 0.63 | 5.00 | 7.9 | B440 |
| 280.9 | | | | 0.21 | 5.00 | 23.8 | B299 |
| 280.125 | | | | 0.15 | 2.29 | 15.8 | B415 |
| 280.5 | | | | 0.26 | 5.00 | 19.2 | B295 |
| 280.146 | | | | 0.09 | 0.29 | 3.1 | B436 |
| 280.104 | | | | 0.09 | 1.16 | 12.8 | B394 |
| 280.65 | | | | 0.07 | 0.50 | 7.2 | B355 |
| 280.44 | | | | 0.10 | 0.54 | 5.6 | B334 |
| 280.149 | | | | 0.07 | 0.17 | 2.4 | B439 |
| 280.157 | | | | 0.16 | 1.79 | 11.1 | B447 |
| 280.151 | | | | 0.17 | 0.95 | 5.6 | B441 |
| 280.85 | | | | 0.22 | 0.92 | 4.1 | B375 |
| 259.112 | 0.25 | 1.76 | 7.0 | | | | B168 |
| 259.101 | 0.65 | 5.00 | 7.7 | | | | B153 |
| 259.115 | 0.32 | 2.38 | 7.3 | | | | B172 |
| 259.55 | 0.41 | 3.43 | 8.3 | | | | B84 |
| 259.178 | 0.29 | 1.45 | 5.1 | | | | B271 |
| 259.98 | 0.15 | 0.88 | 5.9 | | | | B147 |
| 259.3 | 0.23 | 1.46 | 6.4 | | | | B5 |
| 260.3 | 0.10 | 0.69 | 7.2 | | | | B85 |
| 260.21 | 0.22 | 1.98 | 9.1 | | | | B55 |
| 260.28 | 0.16 | 1.62 | 10.2 | | | | B82 |
| 260.79 | 0.17 | 3.33 | 20.1 | | | | B233 |
| 260.51 | 0.26 | 1.97 | 7.6 | | | | B150 |
| 260.5 | 0.24 | 1.47 | 6.1 | | | | B21 |
| 260.58 | 0.50 | 0.51 | 1.0 | | | | B175 |
| 260.22 | 0.07 | 0.78 | 11.2 | | | | B56 |
| 260.13 | 0.08 | 0.57 | 7.6 | | | | B44 |

TABLE 9
Knock down of MYH7 RNA in hIPSC cardiomyocytes (CMs) following 6 days treatment with various concentration
of oligonucleotide. Allele specific RNA was measured using the ddPCR assay. EC50 values are calculated
for each allele and the selectivity was calculated as the ratio between the two EC50 values.

| | EC50 for | EC50 for | | EC50 for | EC50 for | | |
|---|---|---|---|---|---|---|---|
| | perfect match | mismatch to | Selectivity | perfect match | mismatch to | Selectivity | |
| | to c-allele in | t-allele in | between c- and | to t-allele in | c-allele in | between t- and | Compound |
| CMP ID | hIPSC-CM cells | hIPSC-CM cells | t-allele in | hIPSC-CM cells | hIPSC-CM cells | c-allele in | ref. used in |
| NO | (uM) | (uM) | hIPSC-CM cells | (uM) | (uM) | hIPSC-CM cells | examples |
| 259 | 0.05 | 0.28 | 5.1 | | | | A249 |
| 260 | 0.19 | 0.43 | 2.2 | | | | A250 |
| 280 | | | | 0.11 | 0.60 | 5.4 | A270 |
| 280.91 | | | | 0.10 | 0.34 | 3.5 | B381 |
| 280.113 | | | | 0.06 | 0.26 | 4.2 | B403 |
| 280.15 | | | | 0.09 | 0.86 | 9.2 | B440 |
| 280.9 | | | | 0.08 | 0.60 | 7.7 | B299 |
| 280.146 | | | | 0.04 | 0.10 | 2.2 | B436 |
| 280.104 | | | | 0.06 | 0.30 | 4.7 | B394 |
| 280.65 | | | | 0.09 | 0.42 | 4.5 | B355 |
| 280.44 | | | | 0.05 | 0.18 | 3.8 | B334 |
| 259.55 | 0.12 | 0.98 | 7.9 | | | | B84 |
| 259.3 | 0.47 | 5.00 | 10.7 | | | | B5 |
| 260.3 | 0.05 | 0.21 | 4.1 | | | | B85 |
| 260.21 | 0.09 | 0.96 | 11.2 | | | | B55 |
| 260.28 | 0.13 | 1.88 | 14.5 | | | | B82 |
| 260.5 | 0.09 | 0.66 | 7.0 | | | | B21 |
| 260.22 | 0.03 | 0.16 | 5.2 | | | | B56 |
| 260.13 | 0.04 | 0.32 | 7.3 | | | | B44 |

1. An antisense oligonucleotide for the inhibition of a human myosin heavy chain 7 (Myh7) transcript, wherein said oligonucleotide comprises a contiguous nucleotide sequence of 10-30 nucleotides in length which are at least 90% complementary to a sequence selected from the group consisting of SEQ ID NOs 3-10.

2. The antisense oligonucleotide according to claim 1, wherein said oligonucleotide comprises a contiguous nucleotide sequence of 13-24 nucleotides in length which are fully complementary to a sequence selected from the group consisting of SEQ ID NOs 3-10.

3. The antisense oligonucleotide according to claim 1, wherein said antisense oligonucleotide is complementary to a region of the sequence selected from SEQ ID NOs 3-10 which comprises the 20^{th} nucleotide from the 5′ end of the sequence selected from SEQ ID NOs 3-10.

4. The antisense oligonucleotide according to claim 1, wherein the contiguous nucleotide sequence of the oligonucleotide is fully complementary to a sequence selected from the group consisting of SEQ ID NO 3 or SEQ ID NO 4.

5. The antisense oligonucleotide according to claim 1, wherein the contiguous nucleotide sequence of the oligonucleotide is fully complementary to a sequence selected from the group consisting of SEQ ID NO 5 or SEQ ID NO 6.

6. The antisense oligonucleotide according to claim 1, wherein the contiguous nucleotide sequence of the oligonucleotide is fully complementary to a sequence selected from the group consisting of SEQ ID NO 7 or SEQ ID NO 8.

7. The antisense oligonucleotide according to claim 1, wherein the contiguous nucleotide sequence of the oligonucleotide is fully complementary to a sequence selected from the group consisting of SEQ ID NO 9 or SEQ ID NO 10.

8. The antisense oligonucleotide according to claim 1, wherein the Myh7 transcript is the human Myh7 mature mRNA or pre-mRNA.

9. The antisense oligonucleotide according to claim 1, wherein the Myh7 transcript originates from a disease associated allele of the human Myh7 gene.

10. The antisense oligonucleotide according to claim 1, wherein the antisense oligonucleotide is selective for Myh7 transcript originating from a disease associated allele of the human Myh7 transcript, as compared to a non-disease associated allele.

11. The antisense oligonucleotide according to claim 9, wherein the Myh7 transcript originating from a disease associated allele comprises one or more disease associated single nucleotide polymorphisms which are present in a region other than the sequence selected from the group consisting of SEQ ID NO 3-10.

12. The antisense oligonucleotide of claims 1, comprising one or more modified nucleosides.

13. The antisense oligonucleotide of claim 12, wherein the one or more modified nucleosides is a 2′ sugar modified nucleoside.

14. The antisense oligonucleotide of claim 13, wherein the one or more 2′ sugar modified nucleoside is independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

15. The antisense oligonucleotide of claim 12, wherein the one or more modified nucleoside is a LNA nucleoside.

16. The antisense oligonucleotide of claim 1, where the oligonucleotide comprises at least one phosphorothioate internucleoside linkage within the contiguous nucleotide sequence.

17. The antisense oligonucleotide of claim 16, wherein the internucleoside linkages within the contiguous nucleotide sequence are phosphorothioate internucleoside linkages.

18. The antisense oligonucleotide of claim 1, wherein the oligonucleotide is capable of recruiting RNase H.

19. The antisense oligonucleotide of claim 1, wherein the antisense oligonucleotide, or contiguous nucleotide sequence thereof, consists or comprises a gapmer of formula 5′-F-G-F′-3′, where region F and F′ independently comprise 1-8 nucleosides, of which 1-5 are 2′ sugar modified and defines the 5′ and 3′ end of the F and F′ region, and G is a region between 5 and 16 nucleosides which are capable of recruiting RNaseH, such as a region comprising 5-16 DNA nucleosides.

20. The antisense oligonucleotide according to claim 19, wherein region F and F′ comprise at least one LNA nucleoside, and wherein region G comprises 7-14 nucleotides.

21. The antisense oligonucleotide according to claim 1, wherein the antisense oligonucleotide comprises a sequence selected from the group consisting of 11-344.

22. The antisense oligonucleotide according to claim 1, wherein the antisense oligonucleotide consists or comprises of compound ID No 11-344, wherein a capital letter represents a LNA nucleotide, LNA C are LNA 5 methyl cytosine, lower case letters are DNA nucleosides, and optionally all internucleoside linkages are phosphorothioate internucleotides linkages.

23. The antisense oligonucleotide according to claim 1, wherein the antisense oligonucleotide consists or comprises of compound ID No 11-344, wherein a capital letter represents a beta-D-oxy LNA nucleotide, LNA C are LNA 5 methyl cytosine, lower case letters are DNA nucleosides, and all internucleoside linkages are phosphorothioate internucleotides linkages.

24. A conjugate comprising the oligonucleotide according to claim 1, and at least one conjugate moiety covalently attached to said oligonucleotide.

25. A pharmaceutically acceptable salt of the antisense oligonucleotide according to claim 1.

26. A pharmaceutical composition comprising the oligonucleotide of claim 1 and a pharmaceutically acceptable diluent, solvent, carrier, salt and/or adjuvant.

27. An in vivo or in vitro method for modulating human myosin heavy chain 7 (Myh7) expression in a target cell which is expressing Myh7, said method comprising administering an oligonucleotide of claim 1, in an effective amount to said cell.

28. A method for treating or preventing a disease comprising administering a therapeutically or prophylactically effective amount of an oligonucleotide of claim to a subject suffering from or susceptible to the disease.

29. The method of claim 28, wherein the disease is selected from the group consisting of hypertrophic cardiomyopathy.

30. The oligonucleotide of claim 1 for use in medicine.

31. The oligonucleotide of claim 1 for use in the treatment or prevention of hypertrophic cardiomyopathy.

32. Use of the oligonucleotide of claim 1 for the preparation of a medicament for treatment or prevention of hypertrophic cardiomyopathy.

33. A method for treatment of a human subject in need to treatment for hypertrophic cardiomyopathy, said treatment comprising the step of:
a. Taking a biological sample from the human subject
b. Sequencing the Myh7 nucleic acid alleles present in the sample of the human subject;
c. Determining the presence of a disease associated Myh7 allelic variant of the Myh7 nucleic acid;
d. Administering a therapeutically effective amount of an antisense oligonucleotide to the human subject which is selective for the disease associated Myh7 allelic variant as compared to a non-disease associate allele.

34. The method according to claim 33, wherein the antisense oligonucleotide is as according to claim 1.


SEQUENCE LISTING (357 sequences)

SEQ ID NO: 1 - DNA, 22981 bp, homo sapiens
gggggtgggggtgccctgctgccccatatatacagcccctgagaccaggtctggctccacagctctgtcctgctctgtgtctttccctgctgctctcaggtaggagcgggagctggaggctttactctgggataagggggctccaggcttaggaagggattcctcttggaatagcaagcttcatgcaggacttcatgcagagtaccaggtccagtcactgggcacacatgtgcaggtctaaacatgggcgtatgtgccacaggagttcctaggggaagatatctgcatctgagcatatgggaccaatatgcattacagggtgtgtgtgtgtgtgtgtgtgtgtgtgtgtgtgtggacaagcctgcatagtctgaatgtgagaaagggtatgtacctcactgactgatacagaaatatcaagtgggagatgtgggtgtatgatcacgtctgtgaacgaggaggttcctttgcagggagggagaaagaatggtggagaaggattctaggaagtgtgtatgccttcatgtgattctgtgaggcctggaatgtgtagctacccctgtccatgcatgcagaggttcttgtctatgcctccacgagtcatgactgttatagtatctgtaccctctgcagaaggaactcacaggcacgggggtgtaggatgttccaggtctttgtgtactgaagaacacgcagtgatgtgatctacttacgtggcacacatcctgaaatgtgcacatttgctgttgaggcatatatggcaccctggggttggggtgcggattgaggtatgggaaggaagcctgtcttcatgtgagaggcatgcccaagcagcaggcatgagtgttcatcagagggcttgtggagggtgtttggctatgtgatgggtgtgtctgtgagaggaagcccacagtgtcttctctctgagaatgtccacaaagtctgacaacaaaatggggacaggatggaaaagaagtctctttagagtcatcagacttgcattcaagtcttggctctcccacttgttgtcatgggctcttggccaagcaagccatttaacctcgatttcctcatctgtcaaatggggtgaagactgagtaggagaaagtgcattgaagtgcttttagactggaaagtgccgaacaaatgtgaccattagttgtctctcagatggctcaaagaaaggtaccactagatgggtacaggcaggggtgggaccatctcgcaggagtgaagaggattagggttcaggttgagttgacaccaggaagctgttagaactagttcttgaggaaaaagggaatagggtttgaaaaacaagaaggatgggaccagagaagctgaccaccctttccaccttgttttggaagagggtggatgctgaagaacagagccagggagaagcaggaaggtgggactggtcaggttgggcacagcctgccctgacacagcctcttccctctctccaggtcccctgcaggccttggcccctttcctcatctgtagacacacttgagtagcccaggtaagaaaagctgaagctagagtgttgaaaatctagtaagactgggcattagagccccaaagccagcctatggaactctagtttctgccatgtgctggagagaatatcttggctatcaagagctacttacctgtgaccagggggtccagggatagatgagggtcagagatggaaatagttagatttctgctcctacagggagctcagaatgctcttccttcccctaggagagccccagcttgaacaatagttagttgttccttttaccctggcaggtttgctaggctgctcctctggttaaggggtaacctcaaagaggaagggactcactggtaactcctcttgactcttgagcatggtgctaggttttggggctcccactgaaggggagagcccagggagggaagggaagaatgggcagatgggagggcagccagcttctgctcactccaggcacagccatgggagattcggagatggcagtctttggggctgccgccccctacctgcgcaagtcagagaaggagcggctagaagcgcagaccaggccttttgacctcaagaaggatgtcttcgtgcctgatgacaaacaggagtttgtcaaggccaagatcgtgtctcgagagggtggcaaagtcactgccgagaccgagtatggcaaggtgggtgtcaggctgatgtgagagtccaccctggccacctgtacacctgggtggacagagaggggtacacccatgccccatgcccatgcagacctgaggatggtcccattctccttccctttggggaagaatccagaccctccaaagagccctctccttcccaatatcccttctgtcttccctgtgagatcctggttccttctctcttgagcactattgccctgtcactcaccaactcctaaccctcttgaggaaggagggaaagcccaggctgacaggagggcttgggtgggggctcttgcagacagtgaccgtgaaggaggaccaggtgatgcagcagaacccacccaagttcgacaaaatcgaggacatggccatgctgaccttcctgcatgagcccgcggtgctctacaacctcaaggatcgctacggctcctggatgatctacgtgagtgctgcacctggccctacgttgggatctctgttcttgctccatccatgtccaccccaggagccaagagtgtcttctgtttgtgtctaggcagggttacactctaacctcgtcccaacatccttggttcaattccaacactctggggactggcattactcagattgagtgcatgcagaggttttcctttctcttctttctctcctgggatctttctctaactcccaaaatcaccagccctcccccttcgcaactggcaagtcactgctccttttctatccccagacctactcgggcctcttctgtgtcaccgtcaacccttacaagtggctgccggtgtacactcctgaggtggtggctgcctaccggggcaagaagaggagcgaggccccgccccacatcttctccatctccgacaacgcctatcagtacatgctgacaggtgagaggccctggaagggtcttcctgaagggaactgggataggccgggagggagagggagaaggaagggagaagccccacgagagcatcctgtgcagctcctgacctttcctccccaccctctccccacagacagagaaaaccagtccatcctgatcacgtgagtgtagctgctactgtattccctttcagaatctccctgatcccagcctccagccccatcaggtgtcccaggccccgaggcatagactcagcctctttccccccaacctcgtgcccatctccttcttctctgaccattaccctgacccctctctttgctctccctcttcctttctgctttccatccccttcatgtttttcctctcccacctccttcacccactctccaactcatcacgcgtttatccagaagctcattatcgccatgtcttcacctctgcattttctttcccttcagaaacatttcccattcttccagccctaccaacttttcccaaaacagcccaggcccatctcccctgtttactttgcccagccaagcacaggctcagcctgcccaaaccagtgccttctggtggcatccttcctggtgccagccctgacctctgtgcatcagaagacagttgtggattttgggtgtagataacctggcagcttcttgcccagggctcctactctcgggtaggcccaggcattctctcctgatttgaggcttgctggtctccagtagtattgttcactgcccaataagcccctgtcttcacagcggagaatccggagcagggaagacagtcaacaccaagagggtcatccagtactttgctgttattgcagccattggggaccgcagcaagaaggaccagagcccgggcaaggtaggcctgctgccctccaaggtcctgtaccgcagaaagggagggagaagagctctcacctgcctccttcttggcctctgcagggcaccctggaggaccagatcatccaggccaaccctgctctggaggcctttggcaatgccaagaccgtccggaacgacaactcctcccgcttcgtgagtggtccctgaccttggccttgggacttggactggtggaggaatggtctcagatatgagccttcccccaactcatcaccactctcttccatctctccaggggaaattcattcgaattcattttggggcaacaggaaagttggcatctgcagacatagagacctgtgagtgccatgaatctgctaggctcagcctaagctcacccttgctctagaccatctggtcttgacctctctctctctcccctccctccctctgtttttctcctctttaagtctctgtctgtaggtgtctctgtcttcaggtctacatatctgtctctctctgagacttcctctgcatctttctccatttctgtctctgcatggctaggtgtctttctctgggatttctctctgagactatttctctccttctgggtctctgtttccatctctctgtgtgatctctttgtgtctgtccaactagtctctctggctcttcccttccctctgccttttgcttgctacatttatcattaattttccttgtgcccaaaccctaacttttctttctctccttcttctccccacctgttcacagatcttctggaaaaatccagagttattttccagctgaaagcagagagagattatcacattttctaccaaatcctgtctaacaaaaagcctgagctgctgggtgagtcagagccaccgactgagaccaactatctccatggcaacctggtcccctctgcttgtggctggactcagctggcatgccctgggtttcatggcactgctggattcaattcagtgggatgtggggccaagccaagccctgtcctgtgctcttcctcaggccatgtgctgtggcgagcagcctccatgagagccgggggcttgtgtcccaccctaaccatgttttttcccctagacatgctgctgatcaccaacaacccctacgattatgcattcatctcccaaggagagaccaccgtggcctccattgatgacgctgaggagctcatggccactgatgtgagtgtgtgaggacccagccagggggtgggaggattggcagtgaggggcaaacaggggtccaaaagcagagctaagacgctggccattggttgtttaaagtggtatggacctcctgagagccagaatgtgccctgtgatcagggtgtactggggaatggatgaggtgagacaggactggccagtatctggtgttctatcaaaccaggaagagctgaaagccatgagacagagagacacacaagccaagaaacaagcatcaccgagggagcaggacgaggagggaaggccatgcagacggcatggggctggaggggctgttaggtgaacagatgcagacaggtatggaaggccaggcaggaagcaagtgtagggccaggaagcataagtgggtaactcagaaaagctgtggccactgacactggggctgcggccaactgacgtctgctctgctcttgcatcttacatcctttgttttggagttccctgcatcttacatcctttgttttggagctccccactgggtatgggagtctcagaacccacagggattaaggagacaagttttctctccaacttacaagggatctcacttacccatcatacttctttttctggggtccgccaatatggggcctccctacagaacgcttttgatgtgctgggcttcacttcagaggagaaaaactccatgtataagctgacaggcgccatcatgcactttggaaacatgaagttcaagctgaagcagcgggaggagcaggcggagccagacggcactgaaggtgggaggcagggattcttgggggcagctgtcaagtcatggagggccatgtctgctcagcagtcatctctcttctcttttttcttttttttttttttttgagatggagtcttgctctgttgcccaggctggagcgcagtggcacgatcttggctcactgccacctccacatcctgggttcaagtgattctcctgcctcagcctcccgagtagctggaattacaggcatgcaccaccatgcctggctaatttttgtatttttagtagagatggggtttcatcatgttggccaggttggtctcaaactcctgacctcaagtgatccgcctgccttggcctcccaaagtgctgggattacaggcatgaaccaccacacctggccagcagtcatctctttaccaactttgctacttgccttttccttccagaggctgacaagtctgcctacctcatggggctgaactcagccgacctgctcaaggggctgtgccaccctcgggtgaaagtgggcaatgagtacgtcaccaaggggcagaatgtccagcaggtgggtccatcttcagatgataatgggtgggcagggtagggagactggcatggtgggatgagagttttcaagttcactcttcccaacaaccctgctcaatatgggtctctcctccaccttgcaggtgatatatgccactggggcactggccaaggcagtgtatgagaggatgttcaactggatggtgacgcgcatcaatgccaccctggagaccaagcagccacgccagtacttcataggagtcctggacatcgctggcttcgagatcttcgatgtgagttgggacccctgggagtgggagaacaatcactcactcgctcccacattcaacagctatttgcttagagccagctgtggaccagacatgggaaggcagtggggactgtgtggtgacagaggcagtcattttctctgtcttcaggggaagccctccttcactgccttgacatggaggggaccagccacgccctgctgggctcaggcacagtggacgggcacagccccaatggccactcacacccactttctgactgctcccacccctcatgccccctgcagttcaacagctttgagcagctctgcatcaacttcaccaacgagaagctgcagcagttcttcaaccaccacatgtttgtgctggagcaggaggagtacaagaaggagggcatcgagtggacattcattgactttggcatggacctgcaggcctgcattgacctcatcgagaaggtgcctctttggccttaccacctgaattctccctgcacacccaacaagaacaccatagacaaaatagcagcccctctcccttctggggaatataaaaatagaaggggctgaaggatccgagcccttggttccagagcctatgttgtgctgagcacccaggacagggtggcaccagggacagctgatttcccaggggcttgaaagtggacacatggaggatgggcacctgcatgatgacctcccacacctgcatgtttattgggcctggtttatgcatccaaggatcaggaagtgtgagggtggtgtaggggatcataagagtgcacctattttcaaccctagcatctcaggcatctgggtcgtggagtggtgtgtacagcatcgatagaatccatattcccagactttcacaaaggtccttctgtcatcagacaaaatcctccaccttgaaccggttccagtcagtagataactgtactcagagctgagcctactaccttaacacccaacatggcacctccacgagcaagtatattgaccatagagcagaatccatgtccacctgtgtgaaggacactcagtgatgctctctcctgcttcctcagcccatgggcatcatgtccatcctggaagaggagtgcatgttccccaaggccaccgacatgaccttcaaggccaagctgtttgacaaccacctgggcaaatccgccaacttccagaagccacgcaatatcaaggggaagcctgaagcccacttctccctgatccactatgccggcatcgtggactacaacatcattggctggctgcagaagaacaaggatcctctcaatgagactgtcgtgggcttgtatcagaagtcttccctcaagctgctcagcaccctgtttgccaactatgctggggctgatgcgcgtaagtagggactgaggctcccggtacagaggagcagggattctgccaaggttctgagccaggtcaattgctacaccccaccacttcaatgtcaggttgtaccccaaggtttacagactcagtgggatggaactgggtgaagaaactgaggcagccacattgaaggccctcacccaggggccaaatgccagcaaggatgtaaagaggggctgtgattctttactcacaccctacctccccacactgatgcttcttttgttgactctccttcctgcagctattgagaagggcaaaggcaaggccaagaaaggctcgtcctttcagactgtgtcagctctgcacagggtgagtgggacacagccccagccaacttggctccccatctgcccaaccccacccagccccacccttccctgccttgttcatcccctactcctcccgttccctgtctccttggtgcattcgggaccattttcactctgtcttctcttcccgtcatctcctggcctcttcacttatttacttcccatctcccttctctcctctttcccttctgtctcccaccctctctccttatccctgtctgcccccagggccccttcatctctgtgacttctcgaattctctccatctctcttttccttccttcttctcctctcttcttcctgcatctctttctggcattttctttacttttctctattgcatttttggccacaggaaaatctgaacaagctgatgaccaacttgcgctccacccatccccactttgtacgttgtatcatccctaatgagacaaagtctccaggtgaggccacaaactcaggccaacccactgctgggcatacaccgccctgggaacaggacctcctaggacatctccctacctaccaccacagtggtttccaaaccacattcttctcctcccatcccgcatgaggcctcccagggtacctccctctcagcgctaccttgcacttgcagtgtgtaatgagggtgatgtgtaaggcactctgatggacattacctcatcagtggccctacaaccccttggatagctggttgtccaagacagaatcttagttcaactttaaatttacctgcagaaccacttaatcccccaatgagacatattcaaattgttaatccatttttggctaattaggagacagaggctcagggacctgggttcaagacctggctctgttcttgatcagcttagtgaacttgggccagtcccataacccctctgggcttaatttactcatctagaacattggatatatctgttctggctaccacctatgataataggtgtgaatcatataaaaaaggaatgtgaaagccttttagttaaaaatgtggctagaagaaaatgaaacaaatgattattactaactgacttgctaagattacaagctaatcagtgacaaagccaggatcagaacccagaacttcagtccagtgttctcacagactcctcctacttccttcttgccacaggggtgatggacaaccccctggtcatgcaccagctgcgctgcaatggtgtgctggagggcatccgcatctgcaggaaaggcttccccaaccgcatcctctacggggacttccggcagaggtgggtatgagggtgcaccagagctcatagaacagggggagccaggctgccctgatgggaatgggatctgcaggtgaccctggaattctatgggcagagcagatcactgcagagcatgggtgactctggacacttccctcctcaggtatcgcatcctgaacccagcggccatccctgagggacagttcattgatagcaggaagggggcagagaagctgctcagctccctggacattgatcacaaccagtacaagtttggccacaccaaggtgaggaaaggagactaattaattaaaggaagacatctcttttccattgactcctctgatgcttttcctgttgtaatcatcttagcaaaatctcttacctgtatgctacccctcccagtggaacatctagcaccactcccctcatcccagctccagctgccattgacctcctccctgcaatccttttctaggctgttacccttcctaaggtaatccccaccatctctttccctcgtacccctccctagtcatggccaacacacaccttgcctgcaggtgttcttcaaggccgggctgctggggctgctggaggaaatgagggacgagaggctgagccgcatcatcacgcgtatccaggcccagtcccgaggtgtgctcgccagaatggagtacaaaaagctgctggaacgtaggtgagagatctcaagaggaggtttcccgcttctctgaggcccaggctggttcaggggcagtgtcaggaaaaaaagctcagcagatcttcaaacacagagacctgcaggaggggctcatatgaacacactgcagtcacagggtcagaggcctcaggaagggtgggagggatgaaggaaatgggatattcccaaggtttcaggacctcaggtaggaaggaggcaggaggctcagcactcctttcaatgggcccctgcagagactccctgctggtaatccagtggaacattcgggccttcatgggggtcaagaattggccctggatgaagctctacttcaagatcaagccgctgctgaagagtgcagaaagagagaaggagatggcctccatgaaggaggagttcacacgcctcaaagaggcgctagagaagtccgaggctcgccgcaaggagctggaggagaagatggtgtccctgctgcaggagaagaatgacctgcagctccaagtgcaggcggtgaggctcctgggctactgttggctcttccaccctgctctgccttcacttcccacaaccctgcacctccctgcacaggggctcactgtcttgttccttcagtcagaggactctgggatcaacacttctaaagacctccaaagaggtccctcaggaggaggaaagggcagagggaaaagagctgacttttaaaagcacaggctttctacttgggttcaacctagatacacagcttaattattttgtgaccttgggcagaccatgcatcatctttgagcctcaatttcctctagacagggaataaagctgtccatttcattaagccatcatacataagtgcttggcatcagcaaatgcaattcccacccttcgatggaggagaccagaggaagaggtccagatgaagatttctggttcctttcctctctgctcccctccccagtgttcccaagttatactatcctgaaactcttcccctccccatacttctgaagctcttgccaagtggggatatcctagagttaccctcctatttgagtgatgtgcctctccttccctctacctgcaagaatgaggaccttaccccctgaacagcctcccctctgttcctcaccttcaggaacaagacaacctggcagatgctgaggagcgctgtgatcagctgatcaaaaacaagattcagctggaggccaaggtgaaggagatgaacgagaggctggaggatgaggaggagatgaatgctgagctcactgccaagaagcgcaagctggaagatgagtgctcagagctcaaaagggacatcgatgatctggagctgacactggccaaagtggagaaggagaaacacgcaacagagaacaaggtaagggcagctccctttggctccagcccgggtctcatcaggactctcagaccatactgaccttgacccaggctagccactcggcatccagagagcagcatggaccttgacatggagctccccacagatggcaccaagctggtgacctttgaccctaaaggagatgggattcttggtcggcaggtgaaaaacctgacagaggagatggctgggctggatgagatcattgccaagctgaccaaggagaagaaagctctgcaagaggcccaccaacaggctctggatgaccttcaggccgaggaggacaaggtcaacaccctgactaaggccaaagtcaagctggagcagcaagtggatgatgtgagtagattgagagttgtggggcctagatatgccatgtctatctgtgcccagagctctgtgtgtgtgtgtgtgtgtgtgtgtgtgtgtgtgtgtgtgtgtgtgtgtgtttgtgtttatgtttagaggggggtagggttagaggatgttaacttttctgatatctttctagaaaggagctggggaatgaaaagtcagaggtggtgatcaattctgaattatgtatctaatctcactggtatgatcaataatagagatgagttttcttatttaatttaagtgcaatcccatatatctctgcctttgacacaagatttagagggtcatctcataagaacatgataaatttctttggaaattttgtgaggctccccaagtagaatttttgaaattccaatatttaaggcagaatctgatcattgttccctctgatgcacatatgtctgttgactcacttatcactctgtattttaattatttaagcttctatctcagccataagactacaagcttcctttagataggaacttagatctattcaatttggggactgcagtgcttagcacagagcctgatgcccagtaggtgtttataggatgctgaaagaatgaatacggcggccagccgcggtggctcacacctgtaatcccagcactttgggaggccaaggcgggtggatcacgaggtcaagagatcgagaccatcctggccaatatggtgaaaccccatctctactaaaaatacaaaaattagctggatgtggtggcgtgcgcctgtagtcccagctactcaggaggctgaggcagaataatcccttgaaccagggaggcggaggttgcagtgagccgagatcgcgccactgcactccagcctggcgacagagtgagactccataaaaaaaaaaaaaaaaaaaaggaggaaggaaggaaggaaggagagaaagaaaggagagaaagaaagaaagaaagaaagggaaagaaagaaagaatacggctttggggtttcatcccacagtcatttattcctttatctataaaatattgattcagcatcccctatatctaggcaatctcacagtcccctaataacatttggcccactctggggaaggtttcccaagtcctgaacacaaagatttaccaagtcctgaggtaactgaacaacaaaatcaccaagtcaggatgctttgcctcactgggcctggctccttctctctaccagctggaaggatccctggagcaagagaagaaggtgcgcatggacctggagcgagcgaagcggaagctggagggcgacctgaagctgacccaggagagcatcatggacctggagaatgacaagcagcagctggatgagcggctgaaaaagtactgtgtcccctccctgctgcccttcctcctccccagcccataacagtacaagcagacccaagacagccatcctctgaggcaacaacctcacgcagcctccacaagtggaggcgcaaaccatgggaaaaggcctcccaggcaagggctttcctcccctagtcccagatatcagggttagttagatggtcctccctggcaaaaagtccattgtatcccatgagtcctttcttcttgttaggcactcagtgggaatgaaaataacagccttcccccttcatacacatctctctttggagaccagttccttgacatggaggaaggctccacatgagagcctcttaggaaagtccctttgtggcttttcacagagagcccacagggcaccaagaaatgccctcacattaaagaagtcaggggatcagggccatttctttgttttgcttaacttgtggttccaattcttagtgtaagttcttccaaaatgtatagactttccagacttggtaaacagtgagcttgcagaacatattttgataattgaaggtcttatggtgtttcaggctcaggactttgaaaggctttgagaatcatcataatctacatcctcatcaagtttgtgtttgcacaggaatcttcataccttcccaggcacagaatgtgttgaataaatgaataaatacacgatcctatttgattctcaaaacaccttttaaggatggtatctttaatttcactttaccaaagaggaaaataagtttcagaaaggttagctgagttacccaaagcaatagtgacaaaattaaagaccatcagagctaaaaaggacctttgaaacaatctagtccgaccccctcattttacattatgagaaaacaaggaccaaagatgggaatgtcctgtagagcccaggccccatcaccctgtctacagcactcctcccattacattcttgttggatttcaatctcaccgcaggtgttacacttccagggatcctacatcacccccagaagctgtttttctaatgaaatcctactctttacctgtatcattaccattttcaaccactggtggaccctcctgaggccccacgagtctcccttacctcaccatccctccttccccacagaaaagactttgagctgaatgctctcaacgcaaggattgaggatgaacaggccctcggcagccagctgcagaagaagctcaaggagcttcaggtgaggtctggacacccacgcgggatgctgagtccctgggctggttctgtttcccctgccccctcctctagggtgtcctgttcacagcattacaggaactgattatgtgtccatggaggcaggcacgcagctatcacagtgtgcccatggatacatccaaaaggggtcaggtgatccacagagaccctgaaaaggtcacaggcctggagggcagaattcctggcttcctgtctcagctctgccttagttgggtgacctctgggagcccactccagatctctgggtctccatgccctcccctgttacatggaggtttttaagagtccttcgactctgatggcatgactttcctccttactctagagcagaagctgtgttaggtctttagttgtctggtcagacaaatacggctctctcattttcaacttttgttatttttttatgactaaactgcttaaaatatccccagaacacaggtctctttgtccatctttaagagcaagtttcaaccactttccttccaataaagagcatgtagactgtcaggccctcgcctaaaggaaggctcttcaaactatcttccccaaatcctttaattcccacctctccactggaagagctaaactgacttgctgttccagagaagccgagagccttttagagccggggggattccagtggaggggtccaggcggtgggtctgagccctttgtgtctgaccaggcacgcatcgaggagctggaggaggagctggaggccgagcgcaccgccagggctaaggtggagaagctgcgctcagacctgtctcgggagctggaggagatcagcgagcggctggaagaggccggcggggccacgtccgtgcagatcgagatgaacaagaagcgcgaggccgagttccagaagatgcggcgggacctggaggaggccacgctgcagcacgaggccactgccgcggccctgcgcaagaagcacgccgacagcgtggccgagctgggcgagcagatcgacaacctgcagcgggtgaagcagaagctggagaaggagaagagcgagttcaagctggagctggatgacgtcacctccaacatggagcagatcatcaaggccaaggcaggctctgctcggcctcccctcctccaacttcctcctcccacctcccctttctgtccatgtagtgtctcttccttcatggttcattttcccacttcccttcctctgcctggttctcagatcccctccttcttttcacaccttccctcttcttttctccttcagttgcacctcttacaccccttcattccccccgcccccaacatccatcatataactctccttcccttctcaggctaacctggagaagatgtgccggaccttggaagaccagatgaatgagcaccggagcaaggcggaggagacccagcgttctgtcaacgacctcaccagccagcgggccaagttgcaaaccgagaatggtgagcctagagcaggggctccatggttcccaccacagtctccccaacctcctgagccactccagcctcgggcacaagccaatacacgcacacacagacacagagtctctccagaaaatggggcctgagggctagaggaggaggtggggatagagaggagtgctgatctagaatggtgcttccccaggtgagctgtcccggcagctggatgagaaggaggcactgatctcccagctgacccgaggcaagctcacctacacccagcagctggaggacctcaagaggcagctggaggaggaggttaaggtaagggctagactcgggccacctggcccaagcaaggagcacactgactagccttgcatcaaatcacttcccttcccagcaataaggctggctgtggaccaacagttctccaagaattctaaaaagaaatcacagcccaaaccctgccacatgccatctactccccaggctgaggccctctgcttctgctgcatccctggacgggaggcagggtgtgagaaggaggagagaaaatgaactagggaggcacgagatggccttgcccgggcacggctaggggatgtagggactgcacaaatgggtcctgggatttgcagggccacctgaaaactggagaaagaaattcaactggagcctattcctagcactttgctggactcatgaaaaaggagaaagaacatgatttttaaaaaactccagggttcatactaacctgcctcttatagctcagtgatcttaggttagggaagtcccctaacctttctgagcctgtttccctctccccagccaggtaaaatggcggtgatgatgaccatgtctggtgtggggaggagctcagggccaatggccatgaagcttttcttaaatagttcactgctgtattggcattctttgttatcagcaaacagtaaaccatgacaggtccactgggggtttggacatagatgaggccaagggatgatgttgagattggggtgaggagaagggcaagggtggggttgctttatggagaaagctgaacccacctcctggtgcccacccctccccaggcgaagaacgccctggcccacgcactgcagtcggcccggcatgactgcgacctgctgcgggagcagtacgaggaggagacggaggccaaggccgagctgcagcgcgtcctttccaaggccaactcggaggtggcccagtggaggaccaagtatgagacggacgccattcagcggactgaggagctcgaggaggccaagtgagttctgagcagcctgacttctggctgaggcccctttgcaggcaggactcagcccagccccagcctcagcagatcccacacaggcgatgcttagctagtgtttgacaacacaggaggactctgccccggccccacctccttctcctctcagggaagctttttgctcgaattatgtttctgatccgaatataagacgaacaaaaggtttgtctgagggcagagtgcttccgtctggggcagggcactgtggcagggaaaggcagtggggagggctgcagaagcccatacctcctcaatgtccatagcgcagaggctgggccagggtcagagggtgcctgggtctccacgccctgctgggatcctctgcctgatgttctcggcccctgggacctgtcctcaggcttctccagctacacttctgaggtttcaaggattgtctttgagaaggttcatgttgtttcctcttgtccccatccacaccctccatcctccccaccctctgccaccctcccctgggcaggaagaagctggcccagcggctgcaggaagctgaggaggccgtggaggctgttaatgccaagtgctcctcgctggagaagaccaagcaccggctacagaatgagatcgaggacttgatggtggacgtagagcgctccaatgctgctgctgcagccctggacaagaagcagaggaacttcgacaaggtgggccctgggtgggggccgcagccagcatgcagggcaagggggcatgaggggttcagtgagaggccagagccatcctccttggaggtgggggaggagggctgagcccaggcaggtcctgagacagaccctggacatggggctgaggctggggggctgaagagtgagccttgtccccgggcagatcctggccgagtggaagcagaagtatgaggagtcgcagtcggagctggagtcctcgcagaaggaggctcgctccctcagcacagagctcttcaaactcaagaacgcctatgaggagtccctggaacatctggagaccttcaagcgggagaacaaaaacctgcagggtgtgctgggggcccaagaggctggggaggggctgcactggcagtgttcccatatggtgccacccaagggctccaagaggcgtctgggcaaagaggcgtgtccctccaactccactggacctcagcagccctcaaccgagttaccgtgttccccacacagaggagatctccgacttgactgagcagttgggttccagcggaaagactatccatgagctggagaaggtccgaaagcagctggaggccgagaagatggagctgcagtcagccctggaggaggccgaggtgtgtgtgtgtgcagggcacggggtgcagggagctgagctccaggcttttggtccctgttctcatcccccgctttgtccgttttgttcactgtcccatccccagagcctaggacagagcctggcacatagtaggcattcagtgaagatttgtggagtgaatgaatgaccagtcactgaactctcttcacataaacattttacccattgcatcttagctgagcttcccatttccacatacctcccggttcccacctctgcctccctctgttttcttttatactctatacctgattgcctctgtttccttggcaacacttttctcttcattttcctccttgtcttcatagctggctctcccctgtgagggcctgaatcactttctcttaggcttatttttattttctcttagattctcttttcctccaccttctgcttctttgaaccacttacaccactcttgaagtcacttcgtatccatgattagtgagcaggcccccaccctgccctgtgccctgactgtctgcctgcatcccctcccccaaccccttcccaggcctccctggagcacgaggagggcaagatcctccgggcccagctggagttcaaccagatcaaggcagagatcgagcggaagctggcagagaaggacgaggagatggaacaggccaagcgcaaccacctgcgggtggtggactcgctgcagacctccctggacgcagagacacgcagccgcaacgaggccctgagggtgaagaagaagatggaaggagacctcaatgagatggagatccagctcagccacgccaaccgcatggccgccgaggcccagaagcaagtcaagagcctccagagcttgttgaaggtactcacccagaggggactggcctccacgtggcctggcgaagcagtagtgtcttgatacaggcaccagattcctcctgcccctaggttactgcagggacctctgacaggtgcctttagtgaagggaaccgaggctggctccctgctcatgcccactctcctgatcctcaggacacccagattcagctggacgatgcagtccgtgccaacgacgacctgaaggagaacatcgccatcgtggagcggcgcaacaacctgctgcaggctgagctggaggagttgcgtgccgtggtggagcagacagagcggtcccggaagctggcggagcaggagctgattgagactagtgagcgggtgcagctgctgcattcccaggtgagcagctcccctgctcattcctgaagggagcacaggctggggctcagcaagcaaggcttgagagctatgcatagatgctcaatgcttttcctgctctgcccaaccctcccccaacccagaacaccagcctcatcaaccagaagaagaagatggatgctgacctgtcccagctccagactgaagtggaggaggcagtgcaggagtgcaggaatgctgaggagaaggccaagaaggccatcacggatgtaagtcccccactccaccgacccgatccagaccagtgtctctccgtgggctgggcagcaagtgtgtgaggacttgaccagaccatgtgccacctctctcctgcacacaggccgccatgatggcagaggagctgaagaaggagcaggacaccagcgcccacctggagcgcatgaagaagaacatggaacagaccattaaggacctgcagcaccggctggacgaagccgagcagatcgccctcaagggcggcaagaagcagctgcagaagctggaagcgcgggtgcgggagctggagaatgagctggaggccgagcagaagcgcaacgcagagtcggtgaagggcatgaggaagagcgagcggcgcatcaaggagctcacctaccaggtgcgacgggcggtgactccaggcagagccctggcaccatagccacagtgacaaccagctgaggagaatgaagagtttgctcttagcctcttccagggcgaggatgggaatgcagcccccgtttcactttgcctagccctgccccactctgaatgtcccctagctcagaggtcagtctctgagcctcctggcctgggagctccatctcaacacccactcttagcccatcgccaggggacacacacagaattccagacaaagctcacccagtacagctcaccagaaagcaaatatgtagccagggtcacccccaaaagacaccaaaaacacacccatcccattcaaacaagcataaaaactttttcattgcccagggcagtgtggactctagctttctgaggtgttttctagaatcagactctgaattagaatttgtttcttttccaatccaggatgtcactatctctctaagcacactttgtccttaacttagattaagtcagtgttttcctggcctgcattgcagcccaggaaattccatcacactcatagccccaaagtcactcccagcatgtgccgaggtccagagcaggggacttcatcccgcatggggtccccggaccctgaaaaacctgctccctggagcactgcacacacacaattttgtgcataatttcaggagtcccactagttctttggactctttcctggtacagatcatttaaaatatttacacatatttggtaagaaattataaggagaattcaagtgtttagtgaggatcagaaagtagaattgggtcaggatatcagatgaagcagggcagggcggaggggatgctaccttctatgactgtgccatcttcaccccctgcctaccctctggcccccagacggaggaggacaggaaaaacctgctgcggctgcaggacctggtagacaagctgcagctaaaggtcaaggcctacaagcgccaggccgaggaggcggtgagtgaccctgctggggactaggcccaggggaggcataggagagctcgtcccccaagccaggagtctgagaacccaggccccctctcacctcatgctcccacctcccgcaggaggagcaagccaacaccaacctgtccaagttccgcaaggtgcagcacgagctggatgaggcagaggagcgggcggacatcgccgagtcccaggtcaacaagctgcgggccaagagccgtgacattggcacgaaggtgggtccctcttttgggctttgctagtcacccccacagcaggcatacccagacagagcaccctcaaacccgggatgcttccttttcatttattccacacacttgagccacatggccccagaggccaaggtaatgcagttctctgtgatttcaaggattcttccagctctaactttttttttttttttttttgagacggtgtctcactctgccgcccaggctggagtgcagtggcacaatctcagctcactgcaacctctgcctcctgggttctcctgcctcagcctcccaagtagctgggattataggcatgtgctaccatgtctagctaatttttatattttagtagagatgcggtttcaccatgttggccaggctggtcttgaactcctgaccttgtgatccactcgcctcagcctcccaaagtacgggattacaggcgtgagccaccactcccagctcagctctaagtttcagtggttctgggaatttcagccttagggaggggtgccaccctagaagaccctaatttatatacaaccagtatatcccagtttccccccaactgctagtttccagttcccttccacctaattttgtcctgcaatcttgtaactcaaacctaaaatggacatcccagagaacccgggcctggctttgttctcgcccaggcaacttccctctgaccaaccccagaaccctaccctccagaacttgccccccgaacatcgatccctggaaacacatgtgctgctgccacaggggcttccacctgcccacaaagcaggccccccaccatcagacccctctcacctttgttcccatgccctgtccctgcccaataccatctctccaaggactgattggactttgtttcctttcaaaagggcttgaatgaggagtagctttgccacatcttgatctgctcagccctggaggtgccagcaaagccccatgctggagcctgtgtaacagctccttgggaggaagcagaataaagcaattttccttgaagccgaga

SEQ ID NO: 2 - DNA, 6069 bp, homo sapiens
cagcccctgagaccaggtctggctccacagctctgtcctgctctgtgtctttccctgctgctctcaggtcccctgcaggccttggcccctttcctcatctgtagacacacttgagtagcccaggcacagccatgggagattcggagatggcagtctttggggctgccgccccctacctgcgcaagtcagagaaggagcggctagaagcgcagaccaggccttttgacctcaagaaggatgtcttcgtgcctgatgacaaacaggagtttgtcaaggccaagatcgtgtctcgagagggtggcaaagtcactgccgagaccgagtatggcaagacagtgaccgtgaaggaggaccaggtgatgcagcagaacccacccaagttcgacaaaatcgaggacatggccatgctgaccttcctgcatgagcccgcggtgctctacaacctcaaggatcgctacggctcctggatgatctacacctactcgggcctcttctgtgtcaccgtcaacccttacaagtggctgccggtgtacactcctgaggtggtggctgcctaccggggcaagaagaggagcgaggccccgccccacatcttctccatctccgacaacgcctatcagtacatgctgacagacagagaaaaccagtccatcctgatcaccggagaatccggagcagggaagacagtcaacaccaagagggtcatccagtactttgctgttattgcagccattggggaccgcagcaagaaggaccagagcccgggcaagggcaccctggaggaccagatcatccaggccaaccctgctctggaggcctttggcaatgccaagaccgtccggaacgacaactcctcccgcttcgggaaattcattcgaattcattttggggcaacaggaaagttggcatctgcagacatagagacctatcttctggaaaaatccagagttattttccagctgaaagcagagagagattatcacattttctaccaaatcctgtctaacaaaaagcctgagctgctggacatgctgctgatcaccaacaacccctacgattatgcattcatctcccaaggagagaccaccgtggcctccattgatgacgctgaggagctcatggccactgataacgcttttgatgtgctgggcttcacttcagaggagaaaaactccatgtataagctgacaggcgccatcatgcactttggaaacatgaagttcaagctgaagcagcgggaggagcaggcggagccagacggcactgaagaggctgacaagtctgcctacctcatggggctgaactcagccgacctgctcaaggggctgtgccaccctcgggtgaaagtgggcaatgagtacgtcaccaaggggcagaatgtccagcaggtgatatatgccactggggcactggccaaggcagtgtatgagaggatgttcaactggatggtgacgcgcatcaatgccaccctggagaccaagcagccacgccagtacttcataggagtcctggacatcgctggcttcgagatcttcgatttcaacagctttgagcagctctgcatcaacttcaccaacgagaagctgcagcagttcttcaaccaccacatgtttgtgctggagcaggaggagtacaagaaggagggcatcgagtggacattcattgactttggcatggacctgcaggcctgcattgacctcatcgagaagcccatgggcatcatgtccatcctggaagaggagtgcatgttccccaaggccaccgacatgaccttcaaggccaagctgtttgacaaccacctgggcaaatccgccaacttccagaagccacgcaatatcaaggggaagcctgaagcccacttctccctgatccactatgccggcatcgtggactacaacatcattggctggctgcagaagaacaaggatcctctcaatgagactgtcgtgggcttgtatcagaagtcttccctcaagctgctcagcaccctgtttgccaactatgctggggctgatgcgcctattgagaagggcaaaggcaaggccaagaaaggctcgtcctttcagactgtgtcagctctgcacagggaaaatctgaacaagctgatgaccaacttgcgctccacccatccccactttgtacgttgtatcatccctaatgagacaaagtctccaggggtgatggacaaccccctggtcatgcaccagctgcgctgcaatggtgtgctggagggcatccgcatctgcaggaaaggcttccccaaccgcatcctctacggggacttccggcagaggtatcgcatcctgaacccagcggccatccctgagggacagttcattgatagcaggaagggggcagagaagctgctcagctccctggacattgatcacaaccagtacaagtttggccacaccaaggtgttcttcaaggccgggctgctggggctgctggaggaaatgagggacgagaggctgagccgcatcatcacgcgtatccaggcccagtcccgaggtgtgctcgccagaatggagtacaaaaagctgctggaacgtagagactccctgctggtaatccagtggaacattcgggccttcatgggggtcaagaattggccctggatgaagctctacttcaagatcaagccgctgctgaagagtgcagaaagagagaaggagatggcctccatgaaggaggagttcacacgcctcaaagaggcgctagagaagtccgaggctcgccgcaaggagctggaggagaagatggtgtccctgctgcaggagaagaatgacctgcagctccaagtgcaggcggaacaagacaacctggcagatgctgaggagcgctgtgatcagctgatcaaaaacaagattcagctggaggccaaggtgaaggagatgaacgagaggctggaggatgaggaggagatgaatgctgagctcactgccaagaagcgcaagctggaagatgagtgctcagagctcaaaagggacatcgatgatctggagctgacactggccaaagtggagaaggagaaacacgcaacagagaacaaggtgaaaaacctgacagaggagatggctgggctggatgagatcattgccaagctgaccaaggagaagaaagctctgcaagaggcccaccaacaggctctggatgaccttcaggccgaggaggacaaggtcaacaccctgactaaggccaaagtcaagctggagcagcaagtggatgatctggaaggatccctggagcaagagaagaaggtgcgcatggacctggagcgagcgaagcggaagctggagggcgacctgaagctgacccaggagagcatcatggacctggagaatgacaagcagcagctggatgagcggctgaaaaaaaaagactttgagctgaatgctctcaacgcaaggattgaggatgaacaggccctcggcagccagctgcagaagaagctcaaggagcttcaggcacgcatcgaggagctggaggaggagctggaggccgagcgcaccgccagggctaaggtggagaagctgcgctcagacctgtctcgggagctggaggagatcagcgagcggctggaagaggccggcggggccacgtccgtgcagatcgagatgaacaagaagcgcgaggccgagttccagaagatgcggcgggacctggaggaggccacgctgcagcacgaggccactgccgcggccctgcgcaagaagcacgccgacagcgtggccgagctgggcgagcagatcgacaacctgcagcgggtgaagcagaagctggagaaggagaagagcgagttcaagctggagctggatgacgtcacctccaacatggagcagatcatcaaggccaaggctaacctggagaagatgtgccggaccttggaagaccagatgaatgagcaccggagcaaggcggaggagacccagcgttctgtcaacgacctcaccagccagcgggccaagttgcaaaccgagaatggtgagctgtcccggcagctggatgagaaggaggcactgatctcccagctgacccgaggcaagctcacctacacccagcagctggaggacctcaagaggcagctggaggaggaggttaaggcgaagaacgccctggcccacgcactgcagtcggcccggcatgactgcgacctgctgcgggagcagtacgaggaggagacggaggccaaggccgagctgcagcgcgtcctttccaaggccaactcggaggtggcccagtggaggaccaagtatgagacggacgccattcagcggactgaggagctcgaggaggccaagaagaagctggcccagcggctgcaggaagctgaggaggccgtggaggctgttaatgccaagtgctcctcgctggagaagaccaagcaccggctacagaatgagatcgaggacttgatggtggacgtagagcgctccaatgctgctgctgcagccctggacaagaagcagaggaacttcgacaagatcctggccgagtggaagcagaagtatgaggagtcgcagtcggagctggagtcctcgcagaaggaggctcgctccctcagcacagagctcttcaaactcaagaacgcctatgaggagtccctggaacatctggagaccttcaagcgggagaacaaaaacctgcaggaggagatctccgacttgactgagcagttgggttccagcggaaagactatccatgagctggagaaggtccgaaagcagctggaggccgagaagatggagctgcagtcagccctggaggaggccgaggcctccctggagcacgaggagggcaagatcctccgggcccagctggagttcaaccagatcaaggcagagatcgagcggaagctggcagagaaggacgaggagatggaacaggccaagcgcaaccacctgcgggtggtggactcgctgcagacctccctggacgcagagacacgcagccgcaacgaggccctgagggtgaagaagaagatggaaggagacctcaatgagatggagatccagctcagccacgccaaccgcatggccgccgaggcccagaagcaagtcaagagcctccagagcttgttgaaggacacccagattcagctggacgatgcagtccgtgccaacgacgacctgaaggagaacatcgccatcgtggagcggcgcaacaacctgctgcaggctgagctggaggagttgcgtgccgtggtggagcagacagagcggtcccggaagctggcggagcaggagctgattgagactagtgagcgggtgcagctgctgcattcccagaacaccagcctcatcaaccagaagaagaagatggatgctgacctgtcccagctccagactgaagtggaggaggcagtgcaggagtgcaggaatgctgaggagaaggccaagaaggccatcacggatgccgccatgatggcagaggagctgaagaaggagcaggacaccagcgcccacctggagcgcatgaagaagaacatggaacagaccattaaggacctgcagcaccggctggacgaagccgagcagatcgccctcaagggcggcaagaagcagctgcagaagctggaagcgcgggtgcgggagctggagaatgagctggaggccgagcagaagcgcaacgcagagtcggtgaagggcatgaggaagagcgagcggcgcatcaaggagctcacctaccagacggaggaggacaggaaaaacctgctgcggctgcaggacctggtagacaagctgcagctaaaggtcaaggcctacaagcgccaggccgaggaggcggaggagcaagccaacaccaacctgtccaagttccgcaaggtgcagcacgagctggatgaggcagaggagcgggcggacatcgccgagtcccaggtcaacaagctgcgggccaagagccgtgacattggcacgaagggcttgaatgaggagtagctttgccacatcttgatctgctcagccctggaggtgccagcaaagccccatgctggagcctgtgtaacagctccttgggaggaagcagaataaagcaattttccttgaagccgagaaaaaaaaaaaaaaa

SEQ ID NO: 3 - DNA, 39 bp, homo sapiens
agaaaagctgaagctagagtgttgaaaatctagtaagac

SEQ ID NO: 4 - DNA, 39 bp, homo sapiens
agaaaagctgaagctagagcgttgaaaatctagtaagac

SEQ ID NO: 5 - DNA, 39 bp, homo sapiens
gcaaagtcactgccgagaccgagtatggcaagacagtga

SEQ ID NO: 6 - DNA, 39 bp, homo sapiens
gcaaagtcactgccgagactgagtatggcaagacagtga

SEQ ID NO: 7 - DNA, 39 bp, homo sapiens
gcaaagtcactgccgagaccgagtatggcaaggtgggtg

SEQ ID NO: 8 - DNA, 39 bp, homo sapiens
gcaaagtcactgccgagactgagtatggcaaggtgggtg

SEQ ID NO: 9 - DNA, 39 bp, homo sapiens
ctgggctggatgagatcattgccaagctgaccaaggaga

SEQ ID NO: 10 - DNA, 39 bp, homo sapiens
ctgggctggatgagatcatcgccaagctgaccaaggaga

SEQ ID NO: 11 - DNA, 16 bp, artificial
  oligonucleotide
cggtctcggcagtgac

SEQ ID NO: 12 - DNA, 17 bp, artificial
  oligonucleotide
tcggtctcggcagtgac

SEQ ID NO: 13 - DNA, 16 bp, artificial
  oligonucleotide
tcggtctcggcagtga

SEQ ID NO: 14 - DNA, 17 bp, artificial
  oligonucleotide
ctcggtctcggcagtga

SEQ ID NO: 15 - DNA, 19 bp, artificial
  oligonucleotide
tactcggtctcggcagtga

SEQ ID NO: 16 - DNA, 20 bp, artificial
  oligonucleotide
atactcggtctcggcagtga

SEQ ID NO: 17 - DNA, 19 bp, artificial
  oligonucleotide
atactcggtctcggcagtg

SEQ ID NO: 18 - DNA, 20 bp, artificial
  oligonucleotide
catactcggtctcggcagtg

SEQ ID NO: 19 - DNA, 18 bp, artificial
  oligonucleotide
atactcggtctcggcagt

SEQ ID NO: 20 - DNA, 19 bp, artificial
  oligonucleotide
catactcggtctcggcagt

SEQ ID NO: 21 - DNA, 20 bp, artificial
  oligonucleotide
ccatactcggtctcggcagt

SEQ ID NO: 22 - DNA, 16 bp, artificial
  oligonucleotide
tactcggtctcggcag

SEQ ID NO: 23 - DNA, 17 bp, artificial
  oligonucleotide
atactcggtctcggcag

SEQ ID NO: 24 - DNA, 18 bp, artificial
  oligonucleotide
catactcggtctcggcag

SEQ ID NO: 25 - DNA, 19 bp, artificial
  oligonucleotide
ccatactcggtctcggcag

SEQ ID NO: 26 - DNA, 16 bp, artificial
  oligonucleotide
atactcggtctcggca

SEQ ID NO: 27 - DNA, 17 bp, artificial
  oligonucleotide
catactcggtctcggca

SEQ ID NO: 28 - DNA, 18 bp, artificial
  oligonucleotide
ccatactcggtctcggca

SEQ ID NO: 29 - DNA, 15 bp, artificial
  oligonucleotide
atactcggtctcggc

SEQ ID NO: 30 - DNA, 16 bp, artificial
  oligonucleotide
catactcggtctcggc

SEQ ID NO: 31 - DNA, 17 bp, artificial
  oligonucleotide
ccatactcggtctcggc

SEQ ID NO: 32 - DNA, 18 bp, artificial
  oligonucleotide
gccatactcggtctcggc

SEQ ID NO: 33 - DNA, 19 bp, artificial
  oligonucleotide
tgccatactcggtctcggc

SEQ ID NO: 34 - DNA, 20 bp, artificial
  oligonucleotide
ttgccatactcggtctcggc

SEQ ID NO: 35 - DNA, 15 bp, artificial
  oligonucleotide
catactcggtctcgg

SEQ ID NO: 36 - DNA, 16 bp, artificial
  oligonucleotide
ccatactcggtctcgg

SEQ ID NO: 37 - DNA, 17 bp, artificial
  oligonucleotide
gccatactcggtctcgg

SEQ ID NO: 38 - DNA, 18 bp, artificial
  oligonucleotide
tgccatactcggtctcgg

SEQ ID NO: 39 - DNA, 19 bp, artificial
  oligonucleotide
ttgccatactcggtctcgg

SEQ ID NO: 40 - DNA, 20 bp, artificial
  oligonucleotide
cttgccatactcggtctcgg

SEQ ID NO: 41 - DNA, 15 bp, artificial
  oligonucleotide
ccatactcggtctcg

SEQ ID NO: 42 - DNA, 16 bp, artificial
  oligonucleotide
gccatactcggtctcg

SEQ ID NO: 43 - DNA, 17 bp, artificial
  oligonucleotide
tgccatactcggtctcg

SEQ ID NO: 44 - DNA, 18 bp, artificial
  oligonucleotide
ttgccatactcggtctcg

SEQ ID NO: 45 - DNA, 19 bp, artificial
  oligonucleotide
cttgccatactcggtctcg

SEQ ID NO: 46 - DNA, 16 bp, artificial
  oligonucleotide
tgccatactcggtctc

SEQ ID NO: 47 - DNA, 17 bp, artificial
  oligonucleotide
ttgccatactcggtctc

SEQ ID NO: 48 - DNA, 18 bp, artificial
  oligonucleotide
cttgccatactcggtctc

SEQ ID NO: 49 - DNA, 16 bp, artificial
  oligonucleotide
ttgccatactcggtct

SEQ ID NO: 50 - DNA, 17 bp, artificial
  oligonucleotide
cttgccatactcggtct

SEQ ID NO: 51 - DNA, 16 bp, artificial
  oligonucleotide
cttgccatactcggtc

SEQ ID NO: 52 - DNA, 20 bp, artificial
  oligonucleotide
tcttgccatactcggtctcg

SEQ ID NO: 53 - DNA, 19 bp, artificial
  oligonucleotide
tcttgccatactcggtctc

SEQ ID NO: 54 - DNA, 20 bp, artificial
  oligonucleotide
gtcttgccatactcggtctc

SEQ ID NO: 55 - DNA, 18 bp, artificial
  oligonucleotide
tcttgccatactcggtct

SEQ ID NO: 56 - DNA, 19 bp, artificial
  oligonucleotide
gtcttgccatactcggtct

SEQ ID NO: 57 - DNA, 20 bp, artificial
  oligonucleotide
tgtcttgccatactcggtct

SEQ ID NO: 58 - DNA, 17 bp, artificial
  oligonucleotide
tcttgccatactcggtc

SEQ ID NO: 59 - DNA, 18 bp, artificial
  oligonucleotide
gtcttgccatactcggtc

SEQ ID NO: 60 - DNA, 16 bp, artificial
  oligonucleotide
tcttgccatactcggt

SEQ ID NO: 61 - DNA, 17 bp, artificial
  oligonucleotide
gtcttgccatactcggt

SEQ ID NO: 62 - DNA, 16 bp, artificial
  oligonucleotide
gtcttgccatactcgg

SEQ ID NO: 63 - DNA, 17 bp, artificial
  oligonucleotide
tgtcttgccatactcgg

SEQ ID NO: 64 - DNA, 18 bp, artificial
  oligonucleotide
ctgtcttgccatactcgg

SEQ ID NO: 65 - DNA, 20 bp, artificial
  oligonucleotide
cactgtcttgccatactcgg

SEQ ID NO: 66 - DNA, 20 bp, artificial
  oligonucleotide
ccttgccatactcggtctcg

SEQ ID NO: 67 - DNA, 19 bp, artificial
  oligonucleotide
ccttgccatactcggtctc

SEQ ID NO: 68 - DNA, 20 bp, artificial
  oligonucleotide
accttgccatactcggtctc

SEQ ID NO: 69 - DNA, 18 bp, artificial
  oligonucleotide
ccttgccatactcggtct

SEQ ID NO: 70 - DNA, 19 bp, artificial
  oligonucleotide
accttgccatactcggtct

SEQ ID NO: 71 - DNA, 20 bp, artificial
  oligonucleotide
caccttgccatactcggtct

SEQ ID NO: 72 - DNA, 17 bp, artificial
  oligonucleotide
ccttgccatactcggtc

SEQ ID NO: 73 - DNA, 18 bp, artificial
  oligonucleotide
accttgccatactcggtc

SEQ ID NO: 74 - DNA, 19 bp, artificial
  oligonucleotide
caccttgccatactcggtc

SEQ ID NO: 75 - DNA, 20 bp, artificial
  oligonucleotide
ccaccttgccatactcggtc

SEQ ID NO: 76 - DNA, 16 bp, artificial
  oligonucleotide
ccttgccatactcggt

SEQ ID NO: 77 - DNA, 17 bp, artificial
  oligonucleotide
accttgccatactcggt

SEQ ID NO: 78 - DNA, 18 bp, artificial
  oligonucleotide
caccttgccatactcggt

SEQ ID NO: 79 - DNA, 19 bp, artificial
  oligonucleotide
ccaccttgccatactcggt

SEQ ID NO: 80 - DNA, 20 bp, artificial
  oligonucleotide
cccaccttgccatactcggt

SEQ ID NO: 81 - DNA, 16 bp, artificial
  oligonucleotide
accttgccatactcgg

SEQ ID NO: 82 - DNA, 17 bp, artificial
  oligonucleotide
caccttgccatactcgg

SEQ ID NO: 83 - DNA, 18 bp, artificial
  oligonucleotide
ccaccttgccatactcgg

SEQ ID NO: 84 - DNA, 19 bp, artificial
  oligonucleotide
cccaccttgccatactcgg

SEQ ID NO: 85 - DNA, 20 bp, artificial
  oligonucleotide
acccaccttgccatactcgg

SEQ ID NO: 86 - DNA, 18 bp, artificial
  oligonucleotide
tcttgccatactcagtct

SEQ ID NO: 87 - DNA, 18 bp, artificial
  oligonucleotide
ccatactcagtctcggca

SEQ ID NO: 88 - DNA, 18 bp, artificial
  oligonucleotide
ttgccatactcagtctcg

SEQ ID NO: 89 - DNA, 17 bp, artificial
  oligonucleotide
tcttgccatactcagtc

SEQ ID NO: 90 - DNA, 20 bp, artificial
  oligonucleotide
ccatactcagtctcggcagt

SEQ ID NO: 91 - DNA, 16 bp, artificial
  oligonucleotide
ccatactcagtctcgg

SEQ ID NO: 92 - DNA, 19 bp, artificial
  oligonucleotide
cttgccatactcagtctcg

SEQ ID NO: 93 - DNA, 17 bp, artificial
  oligonucleotide
cttgccatactcagtct

SEQ ID NO: 94 - DNA, 20 bp, artificial
  oligonucleotide
ttgccatactcagtctcggc

SEQ ID NO: 95 - DNA, 18 bp, artificial
  oligonucleotide
tgccatactcagtctcgg

SEQ ID NO: 96 - DNA, 18 bp, artificial
  oligonucleotide
ctgtcttgccatactcag

SEQ ID NO: 97 - DNA, 17 bp, artificial
  oligonucleotide
gccatactcagtctcgg

SEQ ID NO: 98 - DNA, 18 bp, artificial
  oligonucleotide
cttgccatactcagtctc

SEQ ID NO: 99 - DNA, 20 bp, artificial
  oligonucleotide
cttgccatactcagtctcgg

SEQ ID NO: 100 - DNA, 19 bp, artificial
  oligonucleotide
ttgccatactcagtctcgg

SEQ ID NO: 101 - DNA, 19 bp, artificial
  oligonucleotide
catactcagtctcggcagt

SEQ ID NO: 102 - DNA, 20 bp, artificial
  oligonucleotide
catactcagtctcggcagtg

SEQ ID NO: 103 - DNA, 17 bp, artificial
  oligonucleotide
tgccatactcagtctcg

SEQ ID NO: 104 - DNA, 19 bp, artificial
  oligonucleotide
atactcagtctcggcagtg

SEQ ID NO: 105 - DNA, 19 bp, artificial
  oligonucleotide
ccatactcagtctcggcag

SEQ ID NO: 106 - DNA, 17 bp, artificial
  oligonucleotide
ccatactcagtctcggc

SEQ ID NO: 107 - DNA, 20 bp, artificial
  oligonucleotide
cactgtcttgccatactcag

SEQ ID NO: 108 - DNA, 18 bp, artificial
  oligonucleotide
atactcagtctcggcagt

SEQ ID NO: 109 - DNA, 18 bp, artificial
  oligonucleotide
gccatactcagtctcggc

SEQ ID NO: 110 - DNA, 20 bp, artificial
  oligonucleotide
tcttgccatactcagtctcg

SEQ ID NO: 111 - DNA, 16 bp, artificial
  oligonucleotide
catactcagtctcggc

SEQ ID NO: 112 - DNA, 16 bp, artificial
  oligonucleotide
tactcagtctcggcag

SEQ ID NO: 113 - DNA, 17 bp, artificial
  oligonucleotide
gtcttgccatactcagt

SEQ ID NO: 114 - DNA, 17 bp, artificial
  oligonucleotide
catactcagtctcggca

SEQ ID NO: 115 - DNA, 17 bp, artificial
  oligonucleotide
atactcagtctcggcag

SEQ ID NO: 116 - DNA, 17 bp, artificial
  oligonucleotide
tgtcttgccatactcag

SEQ ID NO: 117 - DNA, 19 bp, artificial
  oligonucleotide
tgccatactcagtctcggc

SEQ ID NO: 118 - DNA, 16 bp, artificial
  oligonucleotide
gccatactcagtctcg

SEQ ID NO: 119 - DNA, 17 bp, artificial
  oligonucleotide
ttgccatactcagtctc

SEQ ID NO: 120 - DNA, 19 bp, artificial
  oligonucleotide
tcttgccatactcagtctc

SEQ ID NO: 121 - DNA, 19 bp, artificial
  oligonucleotide
actgtcttgccatactcag

SEQ ID NO: 122 - DNA, 16 bp, artificial
  oligonucleotide
atactcagtctcggca

SEQ ID NO: 123 - DNA, 20 bp, artificial
  oligonucleotide
atactcagtctcggcagtga

SEQ ID NO: 124 - DNA, 18 bp, artificial
  oligonucleotide
catactcagtctcggcag

SEQ ID NO: 125 - DNA, 18 bp, artificial
  oligonucleotide
ccttgccatactcagtct

SEQ ID NO: 126 - DNA, 18 bp, artificial
  oligonucleotide
caccttgccatactcagt

SEQ ID NO: 127 - DNA, 17 bp, artificial
  oligonucleotide
ccttgccatactcagtc

SEQ ID NO: 128 - DNA, 19 bp, artificial
  oligonucleotide
ccaccttgccatactcagt

SEQ ID NO: 129 - DNA, 17 bp, artificial
  oligonucleotide
accttgccatactcagt

SEQ ID NO: 130 - DNA, 18 bp, artificial
  oligonucleotide
ccaccttgccatactcag

SEQ ID NO: 131 - DNA, 17 bp, artificial
  oligonucleotide
caccttgccatactcag

SEQ ID NO: 132 - DNA, 20 bp, artificial
  oligonucleotide
acccaccttgccatactcag

SEQ ID NO: 133 - DNA, 18 bp, artificial
  oligonucleotide
accttgccatactcagtc

SEQ ID NO: 134 - DNA, 20 bp, artificial
  oligonucleotide
caccttgccatactcagtct

SEQ ID NO: 135 - DNA, 19 bp, artificial
  oligonucleotide
accttgccatactcagtct

SEQ ID NO: 136 - DNA, 20 bp, artificial
  oligonucleotide
accttgccatactcagtctc

SEQ ID NO: 137 - DNA, 20 bp, artificial
  oligonucleotide
ccttgccatactcagtctcg

SEQ ID NO: 138 - DNA, 20 bp, artificial
  oligonucleotide
cccaccttgccatactcagt

SEQ ID NO: 139 - DNA, 19 bp, artificial
  oligonucleotide
ccttgccatactcagtctc

SEQ ID NO: 140 - DNA, 19 bp, artificial
  oligonucleotide
cccaccttgccatactcag

SEQ ID NO: 141 - DNA, 17 bp, artificial
  oligonucleotide
attttcaacgctctagc

SEQ ID NO: 142 - DNA, 19 bp, artificial
  oligonucleotide
gattttcaacgctctagct

SEQ ID NO: 143 - DNA, 20 bp, artificial
  oligonucleotide
gattttcaacgctctagctt

SEQ ID NO: 144 - DNA, 18 bp, artificial
  oligonucleotide
ctagattttcaacgctct

SEQ ID NO: 145 - DNA, 17 bp, artificial
  oligonucleotide
gattttcaacgctctag

SEQ ID NO: 146 - DNA, 18 bp, artificial
  oligonucleotide
tcaacgctctagcttcag

SEQ ID NO: 147 - DNA, 19 bp, artificial
  oligonucleotide
tttcaacgctctagcttca

SEQ ID NO: 148 - DNA, 20 bp, artificial
  oligonucleotide
ctagattttcaacgctctag

SEQ ID NO: 149 - DNA, 16 bp, artificial
  oligonucleotide
gattttcaacgctcta

SEQ ID NO: 150 - DNA, 19 bp, artificial
  oligonucleotide
tactagattttcaacgctc

SEQ ID NO: 151 - DNA, 16 bp, artificial
  oligonucleotide
ctagattttcaacgct

SEQ ID NO: 152 - DNA, 18 bp, artificial
  oligonucleotide
ttttcaacgctctagctt

SEQ ID NO: 153 - DNA, 17 bp, artificial
  oligonucleotide
ttttcaacgctctagct

SEQ ID NO: 154 - DNA, 17 bp, artificial
  oligonucleotide
tagattttcaacgctct

SEQ ID NO: 155 - DNA, 17 bp, artificial
  oligonucleotide
agattttcaacgctcta

SEQ ID NO: 156 - DNA, 20 bp, artificial
  oligonucleotide
tactagattttcaacgctct

SEQ ID NO: 157 - DNA, 20 bp, artificial
  oligonucleotide
actagattttcaacgctcta

SEQ ID NO: 158 - DNA, 16 bp, artificial
  oligonucleotide
actagattttcaacgc

SEQ ID NO: 159 - DNA, 17 bp, artificial
  oligonucleotide
tttcaacgctctagctt

SEQ ID NO: 160 - DNA, 20 bp, artificial
  oligonucleotide
ttactagattttcaacgctc

SEQ ID NO: 161 - DNA, 19 bp, artificial
  oligonucleotide
ttactagattttcaacgct

SEQ ID NO: 162 - DNA, 20 bp, artificial
  oligonucleotide
attttcaacgctctagcttc

SEQ ID NO: 163 - DNA, 20 bp, artificial
  oligonucleotide
tttcaacgctctagcttcag

SEQ ID NO: 164 - DNA, 19 bp, artificial
  oligonucleotide
agattttcaacgctctagc

SEQ ID NO: 165 - DNA, 18 bp, artificial
  oligonucleotide
actagattttcaacgctc

SEQ ID NO: 166 - DNA, 18 bp, artificial
  oligonucleotide
agattttcaacgctctag

SEQ ID NO: 167 - DNA, 19 bp, artificial
  oligonucleotide
actagattttcaacgctct

SEQ ID NO: 168 - DNA, 19 bp, artificial
  oligonucleotide
ttttcaacgctctagcttc

SEQ ID NO: 169 - DNA, 18 bp, artificial
  oligonucleotide
tttcaacgctctagcttc

SEQ ID NO: 170 - DNA, 18 bp, artificial
  oligonucleotide
ttcaacgctctagcttca

SEQ ID NO: 171 - DNA, 20 bp, artificial
  oligonucleotide
agattttcaacgctctagct

SEQ ID NO: 172 - DNA, 17 bp, artificial
  oligonucleotide
tactagattttcaacgc

SEQ ID NO: 173 - DNA, 19 bp, artificial
  oligonucleotide
ttcaacgctctagcttcag

SEQ ID NO: 174 - DNA, 20 bp, artificial
  oligonucleotide
ttttcaacgctctagcttca

SEQ ID NO: 175 - DNA, 16 bp, artificial
  oligonucleotide
ttttcaacgctctagc

SEQ ID NO: 176 - DNA, 19 bp, artificial
  oligonucleotide
cttactagattttcaacgc

SEQ ID NO: 177 - DNA, 18 bp, artificial
  oligonucleotide
attttcaacgctctagct

SEQ ID NO: 178 - DNA, 18 bp, artificial
  oligonucleotide
tactagattttcaacgct

SEQ ID NO: 179 - DNA, 18 bp, artificial
  oligonucleotide
ttactagattttcaacgc

SEQ ID NO: 180 - DNA, 18 bp, artificial
  oligonucleotide
gattttcaacgctctagc

SEQ ID NO: 181 - DNA, 19 bp, artificial
  oligonucleotide
attttcaacgctctagctt

SEQ ID NO: 182 - DNA, 19 bp, artificial
  oligonucleotide
ctagattttcaacgctcta

SEQ ID NO: 183 - DNA, 17 bp, artificial
  oligonucleotide
caacgctctagcttcag

SEQ ID NO: 184 - DNA, 17 bp, artificial
  oligonucleotide
ttcaacgctctagcttc

SEQ ID NO: 185 - DNA, 17 bp, artificial
  oligonucleotide
actagattttcaacgct

SEQ ID NO: 186 - DNA, 20 bp, artificial
  oligonucleotide
cttactagattttcaacgct

SEQ ID NO: 187 - DNA, 18 bp, artificial
  oligonucleotide
tagattttcaacgctcta

SEQ ID NO: 188 - DNA, 19 bp, artificial
  oligonucleotide
tagattttcaacgctctag

SEQ ID NO: 189 - DNA, 20 bp, artificial
  oligonucleotide
tagattttcaacgctctagc

SEQ ID NO: 190 - DNA, 20 bp, artificial
  oligonucleotide
tcttactagattttcaacgc

SEQ ID NO: 191 - DNA, 17 bp, artificial
  oligonucleotide
ctagattttcaacgctc

SEQ ID NO: 192 - DNA, 16 bp, artificial
  oligonucleotide
ttcaacgctctagctt

SEQ ID NO: 193 - DNA, 20 bp, artificial
  oligonucleotide
cactctagcttcagcttttc

SEQ ID NO: 194 - DNA, 18 bp, artificial
  oligonucleotide
cactctagcttcagcttt

SEQ ID NO: 195 - DNA, 20 bp, artificial
  oligonucleotide
caacactctagcttcagctt

SEQ ID NO: 196 - DNA, 17 bp, artificial
  oligonucleotide
acactctagcttcagct

SEQ ID NO: 197 - DNA, 18 bp, artificial
  oligonucleotide
aacactctagcttcagct

SEQ ID NO: 198 - DNA, 19 bp, artificial
  oligonucleotide
caacactctagcttcagct

SEQ ID NO: 199 - DNA, 20 bp, artificial
  oligonucleotide
tcaacactctagcttcagct

SEQ ID NO: 200 - DNA, 17 bp, artificial
  oligonucleotide
aacactctagcttcagc

SEQ ID NO: 201 - DNA, 18 bp, artificial
  oligonucleotide
caacactctagcttcagc

SEQ ID NO: 202 - DNA, 19 bp, artificial
  oligonucleotide
tcaacactctagcttcagc

SEQ ID NO: 203 - DNA, 20 bp, artificial
  oligonucleotide
ttcaacactctagcttcagc

SEQ ID NO: 204 - DNA, 18 bp, artificial
  oligonucleotide
tcaacactctagcttcag

SEQ ID NO: 205 - DNA, 19 bp, artificial
  oligonucleotide
ttcaacactctagcttcag

SEQ ID NO: 206 - DNA, 20 bp, artificial
  oligonucleotide
tttcaacactctagcttcag

SEQ ID NO: 207 - DNA, 17 bp, artificial
  oligonucleotide
tcaacactctagcttca

SEQ ID NO: 208 - DNA, 18 bp, artificial
  oligonucleotide
ttcaacactctagcttca

SEQ ID NO: 209 - DNA, 19 bp, artificial
  oligonucleotide
tttcaacactctagcttca

SEQ ID NO: 210 - DNA, 20 bp, artificial
  oligonucleotide
ttttcaacactctagcttca

SEQ ID NO: 211 - DNA, 17 bp, artificial
  oligonucleotide
ttcaacactctagcttc

SEQ ID NO: 212 - DNA, 18 bp, artificial
  oligonucleotide
tttcaacactctagcttc

SEQ ID NO: 213 - DNA, 19 bp, artificial
  oligonucleotide
ttttcaacactctagcttc

SEQ ID NO: 214 - DNA, 20 bp, artificial
  oligonucleotide
attttcaacactctagcttc

SEQ ID NO: 215 - DNA, 17 bp, artificial
  oligonucleotide
tttcaacactctagctt

SEQ ID NO: 216 - DNA, 18 bp, artificial
  oligonucleotide
ttttcaacactctagctt

SEQ ID NO: 217 - DNA, 19 bp, artificial
  oligonucleotide
attttcaacactctagctt

SEQ ID NO: 218 - DNA, 20 bp, artificial
  oligonucleotide
gattttcaacactctagctt

SEQ ID NO: 219 - DNA, 17 bp, artificial
  oligonucleotide
ttttcaacactctagct

SEQ ID NO: 220 - DNA, 18 bp, artificial
  oligonucleotide
attttcaacactctagct

SEQ ID NO: 221 - DNA, 19 bp, artificial
  oligonucleotide
gattttcaacactctagct

SEQ ID NO: 222 - DNA, 20 bp, artificial
  oligonucleotide
agattttcaacactctagct

SEQ ID NO: 223 - DNA, 17 bp, artificial
  oligonucleotide
attttcaacactctagc

SEQ ID NO: 224 - DNA, 18 bp, artificial
  oligonucleotide
gattttcaacactctagc

SEQ ID NO: 225 - DNA, 19 bp, artificial
  oligonucleotide
agattttcaacactctagc

SEQ ID NO: 226 - DNA, 20 bp, artificial
  oligonucleotide
tagattttcaacactctagc

SEQ ID NO: 227 - DNA, 17 bp, artificial
  oligonucleotide
gattttcaacactctag

SEQ ID NO: 228 - DNA, 18 bp, artificial
  oligonucleotide
agattttcaacactctag

SEQ ID NO: 229 - DNA, 19 bp, artificial
  oligonucleotide
tagattttcaacactctag

SEQ ID NO: 230 - DNA, 20 bp, artificial
  oligonucleotide
ctagattttcaacactctag

SEQ ID NO: 231 - DNA, 17 bp, artificial
  oligonucleotide
agattttcaacactcta

SEQ ID NO: 232 - DNA, 18 bp, artificial
  oligonucleotide
tagattttcaacactcta

SEQ ID NO: 233 - DNA, 19 bp, artificial
  oligonucleotide
ctagattttcaacactcta

SEQ ID NO: 234 - DNA, 20 bp, artificial
  oligonucleotide
actagattttcaacactcta

SEQ ID NO: 235 - DNA, 17 bp, artificial
  oligonucleotide
tagattttcaacactct

SEQ ID NO: 236 - DNA, 18 bp, artificial
  oligonucleotide
ctagattttcaacactct

SEQ ID NO: 237 - DNA, 19 bp, artificial
  oligonucleotide
actagattttcaacactct

SEQ ID NO: 238 - DNA, 20 bp, artificial
  oligonucleotide
tactagattttcaacactct

SEQ ID NO: 239 - DNA, 16 bp, artificial
  oligonucleotide
tagattttcaacactc

SEQ ID NO: 240 - DNA, 17 bp, artificial
  oligonucleotide
ctagattttcaacactc

SEQ ID NO: 241 - DNA, 18 bp, artificial
  oligonucleotide
actagattttcaacactc

SEQ ID NO: 242 - DNA, 19 bp, artificial
  oligonucleotide
tactagattttcaacactc

SEQ ID NO: 243 - DNA, 20 bp, artificial
  oligonucleotide
ttactagattttcaacactc

SEQ ID NO: 244 - DNA, 17 bp, artificial
  oligonucleotide
actagattttcaacact

SEQ ID NO: 245 - DNA, 18 bp, artificial
  oligonucleotide
tactagattttcaacact

SEQ ID NO: 246 - DNA, 19 bp, artificial
  oligonucleotide
ttactagattttcaacact

SEQ ID NO: 247 - DNA, 20 bp, artificial
  oligonucleotide
cttactagattttcaacact

SEQ ID NO: 248 - DNA, 17 bp, artificial
  oligonucleotide
tactagattttcaacac

SEQ ID NO: 249 - DNA, 18 bp, artificial
  oligonucleotide
ttactagattttcaacac

SEQ ID NO: 250 - DNA, 19 bp, artificial
  oligonucleotide
cttactagattttcaacac

SEQ ID NO: 251 - DNA, 20 bp, artificial
  oligonucleotide
tcttactagattttcaacac

SEQ ID NO: 252 - DNA, 18 bp, artificial
  oligonucleotide
cagcttggcgatgatctc

SEQ ID NO: 253 - DNA, 19 bp, artificial
  oligonucleotide
agcttggcgatgatctcat

SEQ ID NO: 254 - DNA, 17 bp, artificial
  oligonucleotide
tcagcttggcgatgatc

SEQ ID NO: 255 - DNA, 16 bp, artificial
  oligonucleotide
cagcttggcgatgatc

SEQ ID NO: 256 - DNA, 20 bp, artificial
  oligonucleotide
tcagcttggcgatgatctca

SEQ ID NO: 257 - DNA, 20 bp, artificial
  oligonucleotide
cagcttggcgatgatctcat

SEQ ID NO: 258 - DNA, 17 bp, artificial
  oligonucleotide
cttggcgatgatctcat

SEQ ID NO: 259 - DNA, 17 bp, artificial
  oligonucleotide
cagcttggcgatgatct

SEQ ID NO: 260 - DNA, 18 bp, artificial
  oligonucleotide
tcagcttggcgatgatct

SEQ ID NO: 261 - DNA, 19 bp, artificial
  oligonucleotide
tcagcttggcgatgatctc

SEQ ID NO: 262 - DNA, 17 bp, artificial
  oligonucleotide
gcttggcgatgatctca

SEQ ID NO: 263 - DNA, 17 bp, artificial
  oligonucleotide
agcttggcgatgatctc

SEQ ID NO: 264 - DNA, 18 bp, artificial
  oligonucleotide
agcttggcgatgatctca

SEQ ID NO: 265 - DNA, 19 bp, artificial
  oligonucleotide
cagcttggcgatgatctca

SEQ ID NO: 266 - DNA, 18 bp, artificial
  oligonucleotide
gcttggcgatgatctcat

SEQ ID NO: 267 - DNA, 17 bp, artificial
  oligonucleotide
caatgatctcatccagc

SEQ ID NO: 268 - DNA, 18 bp, artificial
  oligonucleotide
gcaatgatctcatccagc

SEQ ID NO: 269 - DNA, 19 bp, artificial
  oligonucleotide
ggcaatgatctcatccagc

SEQ ID NO: 270 - DNA, 20 bp, artificial
  oligonucleotide
tggcaatgatctcatccagc

SEQ ID NO: 271 - DNA, 17 bp, artificial
  oligonucleotide
gcaatgatctcatccag

SEQ ID NO: 272 - DNA, 18 bp, artificial
  oligonucleotide
ggcaatgatctcatccag

SEQ ID NO: 273 - DNA, 19 bp, artificial
  oligonucleotide
tggcaatgatctcatccag

SEQ ID NO: 274 - DNA, 17 bp, artificial
  oligonucleotide
ggcaatgatctcatcca

SEQ ID NO: 275 - DNA, 18 bp, artificial
  oligonucleotide
tggcaatgatctcatcca

SEQ ID NO: 276 - DNA, 19 bp, artificial
  oligonucleotide
ttggcaatgatctcatcca

SEQ ID NO: 277 - DNA, 20 bp, artificial
  oligonucleotide
cttggcaatgatctcatcca

SEQ ID NO: 278 - DNA, 17 bp, artificial
  oligonucleotide
tggcaatgatctcatcc

SEQ ID NO: 279 - DNA, 18 bp, artificial
  oligonucleotide
ttggcaatgatctcatcc

SEQ ID NO: 280 - DNA, 19 bp, artificial
  oligonucleotide
cttggcaatgatctcatcc

SEQ ID NO: 281 - DNA, 20 bp, artificial
  oligonucleotide
gcttggcaatgatctcatcc

SEQ ID NO: 282 - DNA, 17 bp, artificial
  oligonucleotide
ttggcaatgatctcatc

SEQ ID NO: 283 - DNA, 18 bp, artificial
  oligonucleotide
cttggcaatgatctcatc

SEQ ID NO: 284 - DNA, 19 bp, artificial
  oligonucleotide
gcttggcaatgatctcatc

SEQ ID NO: 285 - DNA, 20 bp, artificial
  oligonucleotide
agcttggcaatgatctcatc

SEQ ID NO: 286 - DNA, 18 bp, artificial
  oligonucleotide
gcttggcaatgatctcat

SEQ ID NO: 287 - DNA, 19 bp, artificial
  oligonucleotide
agcttggcaatgatctcat

SEQ ID NO: 288 - DNA, 20 bp, artificial
  oligonucleotide
cagcttggcaatgatctcat

SEQ ID NO: 289 - DNA, 17 bp, artificial
  oligonucleotide
gcttggcaatgatctca

SEQ ID NO: 290 - DNA, 18 bp, artificial
  oligonucleotide
agcttggcaatgatctca

SEQ ID NO: 291 - DNA, 19 bp, artificial
  oligonucleotide
cagcttggcaatgatctca

SEQ ID NO: 292 - DNA, 20 bp, artificial
  oligonucleotide
tcagcttggcaatgatctca

SEQ ID NO: 293 - DNA, 17 bp, artificial
  oligonucleotide
agcttggcaatgatctc

SEQ ID NO: 294 - DNA, 18 bp, artificial
  oligonucleotide
cagcttggcaatgatctc

SEQ ID NO: 295 - DNA, 19 bp, artificial
  oligonucleotide
tcagcttggcaatgatctc

SEQ ID NO: 296 - DNA, 17 bp, artificial
  oligonucleotide
cagcttggcaatgatct

SEQ ID NO: 297 - DNA, 18 bp, artificial
  oligonucleotide
tcagcttggcaatgatct

SEQ ID NO: 298 - DNA, 17 bp, artificial
  oligonucleotide
tcagcttggcaatgatc

SEQ ID NO: 299 - DNA, 17 bp, artificial
  oligonucleotide
tcgaggttaaatggctt

SEQ ID NO: 300 - DNA, 18 bp, artificial
  oligonucleotide
atcgaggttaaatggctt

SEQ ID NO: 301 - DNA, 17 bp, artificial
  oligonucleotide
ggtcagggtaatggtca

SEQ ID NO: 302 - DNA, 18 bp, artificial
  oligonucleotide
aaacatgaaggggatgga

SEQ ID NO: 303 - DNA, 18 bp, artificial
  oligonucleotide
ggaaatgtttctgaaggg

SEQ ID NO: 304 - DNA, 18 bp, artificial
  oligonucleotide
gaatgggaaatgtttctg

SEQ ID NO: 305 - DNA, 17 bp, artificial
  oligonucleotide
aagttggtagggctgga

SEQ ID NO: 306 - DNA, 17 bp, artificial
  oligonucleotide
aaagttggtagggctgg

SEQ ID NO: 307 - DNA, 18 bp, artificial
  oligonucleotide
aaaagttggtagggctgg

SEQ ID NO: 308 - DNA, 17 bp, artificial
  oligonucleotide
aaaagttggtagggctg

SEQ ID NO: 309 - DNA, 18 bp, artificial
  oligonucleotide
gaaaagttggtagggctg

SEQ ID NO: 310 - DNA, 17 bp, artificial
  oligonucleotide
gaaaagttggtagggct

SEQ ID NO: 311 - DNA, 18 bp, artificial
  oligonucleotide
ggaaaagttggtagggct

SEQ ID NO: 312 - DNA, 18 bp, artificial
  oligonucleotide
agagacttaaagaggaga

SEQ ID NO: 313 - DNA, 16 bp, artificial
  oligonucleotide
ggttgttggtgatcag

SEQ ID NO: 314 - DNA, 16 bp, artificial
  oligonucleotide
atgcataatcgtaggg

SEQ ID NO: 315 - DNA, 17 bp, artificial
  oligonucleotide
aaaggatgtaagatgca

SEQ ID NO: 316 - DNA, 16 bp, artificial
  oligonucleotide
aaaggatgtaagatgc

SEQ ID NO: 317 - DNA, 17 bp, artificial
  oligonucleotide
acaaaggatgtaagatg

SEQ ID NO: 318 - DNA, 18 bp, artificial
  oligonucleotide
aacaaaggatgtaagatg

SEQ ID NO: 319 - DNA, 18 bp, artificial
  oligonucleotide
ctattttgtctatggtgt

SEQ ID NO: 320 - DNA, 17 bp, artificial
  oligonucleotide
ctattttgtctatggtg

SEQ ID NO: 321 - DNA, 17 bp, artificial
  oligonucleotide
caggtggttgtcaaaca

SEQ ID NO: 322 - DNA, 18 bp, artificial
  oligonucleotide
cattgaagtggtggggtg

SEQ ID NO: 323 - DNA, 18 bp, artificial
  oligonucleotide
gatggagagaattcgaga

SEQ ID NO: 324 - DNA, 17 bp, artificial
  oligonucleotide
cgtacaaagtggggatg

SEQ ID NO: 325 - DNA, 18 bp, artificial
  oligonucleotide
acgtacaaagtggggatg

SEQ ID NO: 326 - DNA, 16 bp, artificial
  oligonucleotide
cgtacaaagtggggat

SEQ ID NO: 327 - DNA, 16 bp, artificial
  oligonucleotide
acgtacaaagtgggga

SEQ ID NO: 328 - DNA, 16 bp, artificial
  oligonucleotide
aacgtacaaagtgggg

SEQ ID NO: 329 - DNA, 18 bp, artificial
  oligonucleotide
agtaggaggagtctgtga

SEQ ID NO: 330 - DNA, 18 bp, artificial
  oligonucleotide
aagtaggaggagtctgtg

SEQ ID NO: 331 - DNA, 18 bp, artificial
  oligonucleotide
gaagtaggaggagtctgt

SEQ ID NO: 332 - DNA, 18 bp, artificial
  oligonucleotide
ggaagtaggaggagtctg

SEQ ID NO: 333 - DNA, 18 bp, artificial
  oligonucleotide
ggaggggaagagtttcag

SEQ ID NO: 334 - DNA, 18 bp, artificial
  oligonucleotide
tcttgcaggtagagggaa

SEQ ID NO: 335 - DNA, 17 bp, artificial
  oligonucleotide
gagtgataagtgagtca

SEQ ID NO: 336 - DNA, 18 bp, artificial
  oligonucleotide
ttattaggggactgtgag

SEQ ID NO: 337 - DNA, 18 bp, artificial
  oligonucleotide
gaaggctgttattttcat

SEQ ID NO: 338 - DNA, 18 bp, artificial
  oligonucleotide
ggaaggctgttattttca

SEQ ID NO: 339 - DNA, 18 bp, artificial
  oligonucleotide
aggaggggatctgagaac

SEQ ID NO: 340 - DNA, 18 bp, artificial
  oligonucleotide
aaggaggggatctgagaa

SEQ ID NO: 341 - DNA, 17 bp, artificial
  oligonucleotide
aggcgttcttgagtttg

SEQ ID NO: 342 - DNA, 17 bp, artificial
  oligonucleotide
aaatgatctgtaccagg

SEQ ID NO: 343 - DNA, 18 bp, artificial
  oligonucleotide
aagttctggagggtaggg

SEQ ID NO: 344 - DNA, 17 bp, artificial
  oligonucleotide
tgagaggggtctgatgg

SEQ ID NO: 345 - DNA, 18 bp, artificial
  oligonucleotide
tgcagagctttcttctcc

SEQ ID NO: 346 - DNA, 18 bp, artificial
  oligonucleotide
cagcttggcaatgatctc

SEQ ID NO: 347 - DNA, 16 bp, artificial
  oligonucleotide
cagaggagatggctgg

SEQ ID NO: 348 - DNA, 18 bp, artificial
  oligonucleotide
tgcagagctttcttctcc

SEQ ID NO: 349 - DNA, 17 bp, artificial
  oligonucleotide
cagcttggcgatgatct

SEQ ID NO: 350 - DNA, 20 bp, artificial
  oligonucleotide
aaaacctaacagaggagatg

SEQ ID NO: 351 - DNA, 19 bp, artificial
  oligonucleotide
cttcttgcagagctttctt

SEQ ID NO: 352 - DNA, 17 bp, artificial
  oligonucleotide
tgagatcatcgccaagc

SEQ ID NO: 353 - DNA, 20 bp, artificial
  oligonucleotide
aaaacctaacagaggagatg

SEQ ID NO: 354 - DNA, 19 bp, artificial
  oligonucleotide
cttcttgcagagctttctt

SEQ ID NO: 355 - DNA, 19 bp, artificial
  oligonucleotide
tgaaatcattgccaagctg

SEQ ID NO: 356 - DNA, 57 bp, artificial
  oligonucleotide
acagaggagatggctgggctggatgagatcatcgccaagctgaccaaggagaagaaa

SEQ ID NO: 357 - DNA, 57 bp, artificial
  oligonucleotide
acagaggagatggctgggctggatgaaatcattgccaagctgaccaaagagaagaaa